SEQUENCE LISTING (83 sequences)

SEQ ID NO: 1 - protein, 7 aa, Artificial Sequence
  CDR-H1
SDTTAWS

SEQ ID NO: 2 - protein, 7 aa, Artificial Sequence
  CDR-H1
SNSAAWN

SEQ ID NO: 3 - protein, 5 aa, Artificial Sequence
  CDR-H1
SSAMH

SEQ ID NO: 4 - protein, 18 aa, Artificial Sequence
  CDR-H2
WTYYRSKWFNYYAVSVKG

SEQ ID NO: 5 - protein, 18 aa, Artificial Sequence
  CDR-H2
WTYYRSKWFNYYAVSVKS

SEQ ID NO: 6 - protein, 18 aa, Artificial Sequence
  CDR-H2
RTYYRSKWYNDYAVSVKS

SEQ ID NO: 7 - protein, 17 aa, Artificial Sequence
  CDR-H2
AISYDGSDTYYADSVKG

SEQ ID NO: 8 - protein, 18 aa, Artificial Sequence
  CDR-H2
WIYYRSKWFNYYAVSVKG

SEQ ID NO: 9 - protein, 18 aa, Artificial Sequence
  CDR-H2
WTYYRSAWFNYYAVSVKG

SEQ ID NO: 10 - protein, 18 aa, Artificial Sequence
  CDR-H2
WTYYRSKWANYYAVSVKG

SEQ ID NO: 11 - protein, 9 aa, Artificial Sequence
  CDR-H3
GNGNGGMDV

SEQ ID NO: 12 - protein, 8 aa, Artificial Sequence
  CDR-H3
GSYGSGLV

SEQ ID NO: 13 - protein, 11 aa, Artificial Sequence
  CDR-H3
DQVLGSGSLDY

SEQ ID NO: 14 - protein, 9 aa, Artificial Sequence
  CDR-H3
GNGAGGMDV

SEQ ID NO: 15 - protein, 11 aa, Artificial Sequence
  CDR-L1
SGDALPKQYAY

SEQ ID NO: 16 - protein, 13 aa, Artificial Sequence
  CDR-L1
TGSSGSIASNYVQ

SEQ ID NO: 17 - protein, 13 aa, Artificial Sequence
  CDR-L1
TRSSGSIGSYYVQ

SEQ ID NO: 18 - protein, 11 aa, Artificial Sequence
  CDR-L1
SGDALPAQYAY

SEQ ID NO: 19 - protein, 7 aa, Artificial Sequence
  CDR-L2
KDSERPS

SEQ ID NO: 20 - protein, 7 aa, Artificial Sequence
  CDR-L2
EDDQRPS

SEQ ID NO: 21 - protein, 7 aa, Artificial Sequence
  CDR-L2
EDDKRPS

SEQ ID NO: 22 - protein, 7 aa, Artificial Sequence
  CDR-L2
ADSERPS

SEQ ID NO: 23 - protein, 11 aa, Artificial Sequence
  CDR-L3
QSVDNSGFYQV

SEQ ID NO: 24 - protein, 11 aa, Artificial Sequence
  CDR-L3
QSADNSGFYQV

SEQ ID NO: 25 - protein, 10 aa, Artificial Sequence
  CDR-L3
QSYDNKDNVV

SEQ ID NO: 26 - protein, 9 aa, Artificial Sequence
  CDR-L3
QSYDSSHVV

SEQ ID NO: 27 - protein, 11 aa, Artificial Sequence
  CDR-L3
QSVDNSGFAQV

SEQ ID NO: 28 - protein, 121 aa, Artificial Sequence
  Clone 10A1 clone VH
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSS

SEQ ID NO: 29 - protein, 108 aa, Artificial Sequence
  Clone 10A1 clone VL
SYVLTQPASASVSPGQTARITCSGDALPKQYAYWYQQKPGQAPVLVIFKDSERPSGVPERFSGSSSGTTVTLTISGAQAEDEADYYCQSVDNSGFYQVFGGGTQLTVL

SEQ ID NO: 30 - protein, 121 aa, Artificial Sequence
  Clone 10 VH
QVQLQQSGPVLVKPSQTLSLTCAISGDSVSSDTTAWSWIRQSPSRGLEWLGWTYYRSKWFNYYAVSVKSRVTINPDTSKNQFSLQLSSVTPEDTAVYYCARGNGNGGMDVWGQGTTVTVSS

SEQ ID NO: 31 - protein, 108 aa, Artificial Sequence
  Clone 10 VL
SYVLTQPPSVSVSPGQTARITCSGDALPKQYAYWYQQKPGQAPVLVIFKDSERPSGIPERFSGSSSGTTVTLTISGVQAEDEADYYCQSADNSGFYQVFGGGTQLTVL

SEQ ID NO: 32 - protein, 120 aa, Artificial Sequence
  Clone 22 VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGSYGSGLVWGQGTTVTVSS

SEQ ID NO: 33 - protein, 111 aa, Artificial Sequence
  Clone 22 VL
NFMLTQPHSVSESPGKTVTISCTGSSGSIASNYVQWYQQRPGSAPTTVIYEDDQRPSGVPDRFSGSLDTSSNSASLTISGLRPEDEADYYCQSYDNKDNVVFGGGTKVTVL

SEQ ID NO: 34 - protein, 120 aa, Artificial Sequence
  Clone 30 VH
EVQLLETGGGLVQPGGSLRLSCAASGFTFSSSAMHWVRQAPGKGLEWVAAISYDGSDTYYADSVKGRFTISRDNAKNTLYLQMNSLRDEDTAVYYCARDQVLGSGSLDYWGQGTLVTVSS

SEQ ID NO: 35 - protein, 110 aa, Artificial Sequence
  Clone 30 VL
NFMLTQPHSVSESPGKTVTISCTRSSGSIGSYYVQWYQQRPGSPPTTVIYEDDKRPSGVPDRFSGSIDSSSNSASLTISGLKTEDEADYYCQSYDSSHVVFGGGTKLTVL

SEQ ID NO: 36 - protein, 121 aa, Artificial Sequence
  Clone 10A1 VH_S15G
QVQLQQSGPVLVKPGQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSS

SEQ ID NO: 37 - protein, 121 aa, Artificial Sequence
  Clone 10A1 VH_T53I
QVQLQQSGPVLVKPSQTLSLTCAISGDSISSDTTAWSWIRRSPSRGLEWLGWIYYRSKWFNYYAVSVKGRVTINPDTSKNQFSLQLSSVTPEDTAVYYCARGNGNGGMDVWGQGTTVTVSS

SEQ ID NO: 38 - protein, 121 aa, Artificial Sequence
  Clone 10A1 VH_K58A
QVQLQQSGPVLVKPSQTLSLTCAISGDSISSDTTAWSWIRRSPSRGLEWLGWTYYRSAWFNYYAVSVKGRVTINPDTSKNQFSLQLSSVTPEDTAVYYCARGNGNGGMDVWGQGTTVTVSS

SEQ ID NO: 39 - protein, 121 aa, Artificial Sequence
  Clone 10A1 VH_F60A
QVQLQQSGPVLVKPSQTLSLTCAISGDSISSDTTAWSWIRRSPSRGLEWLGWTYYRSKWANYYAVSVKGRVTINPDTSKNQFSLQLSSVTPEDTAVYYCARGNGNGGMDVWGQGTTVTVSS

SEQ ID NO: 40 - protein, 121 aa, Artificial Sequence
  Clone 10A1 VH_N105A
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGAGGMDVWGQGTTVTVSS

SEQ ID NO: 41 - protein, 121 aa, Artificial Sequence
  Clone 10A1 VH_S15G/T53I/N105A
QVQLQQSGPVLVKPGQTLSLTCAISGDSISSDTTAWSWIRRSPSRGLEWLGWIYYRSKWFNYYAVSVKGRVTINPDTSKNQFSLQLSSVTPEDTAVYYCARGNGAGGMDVWGQGTTVTVSS

SEQ ID NO: 42 - protein, 108 aa, Artificial Sequence
  Clone 10A1 VL_K29A
SYVLTQPASASVSPGQTARITCSGDALPAQYAYWYQQKPGQAPVLVIFKDSERPSGVPERFSGSSSGTTVTLTISGAQAEDEADYYCQSVDNSGFYQVFGGGTQLTVL

SEQ ID NO: 43 - protein, 108 aa, Artificial Sequence
  Clone 10A1 VL_K49A
SYVLTQPASASVSPGQTARITCSGDALPKQYAYWYQQKPGQAPVLVIFADSERPSGVPERFSGSSSGTTVTLTISGAQAEDEADYYCQSVDNSGFYQVFGGGTQLTVL

SEQ ID NO: 44 - protein, 108 aa, Artificial Sequence
  Clone 10A1 VL_Y96A
SYVLTQPASASVSPGQTARITCSGDALPKQYAYWYQQKPGQAPVLVIFKDSERPSGVPERFSGSSSGTTVTLTISGAQAEDEADYYCQSVDNSGFAQVFGGGTQLTVL

SEQ ID NO: 45 - protein, 451 aa, Artificial Sequence
  Clone 10A1 LALA Heavy Chain
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 710 aa, Artificial Sequence
  Clone 10A1 HC-scFv
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSGSSGSSSSSGSSSQVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSGSSSSSSGSSGSSSSSYVLTQPASASVSPGQTARITCSGDALPKQYAYWYQQKPGQAPVLVIFKDSERPSGVPERFSGSSSGTTVTLTISGAQAEDEADYYCQSVDNSGFYQVFGGGTQLTVL

SEQ ID NO: 47 - protein, 451 aa, Artificial Sequence
  Clone 10A1 E345R Heavy Chain
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 451 aa, Artificial Sequence
  Clone 10A1 RGY Heavy Chain
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 49 - protein, 215 aa, Artificial Sequence
  Clone 10A1 kappa light chain
SYVLTQPASASVSPGQTARITCSGDALPKQYAYWYQQKPGQAPVLVIFKDSERPSGVPERFSGSSSGTTVTLTISGAQAEDEADYYCQSVDNSGFYQVFGGGTQLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 504 aa, Artificial Sequence
  Clone 10A1 scFv-Fc
QVQLQQSGPVLVKPSQTLSLTCAISGDSISSDTTAWSWIRRSPSRGLEWLGWTYYRSKWFNYYAVSVKGRVTINPDTSKNQFSLQLSSVTPEDTAVYYCARGNGNGGMDVWGQGTTVTVSSGILSGSSSSGSSSSGSSSSGYVLTQPASASVSPGQTARITCSGDALPKQYAYWYQQKPGQAPVLVIFKDSERPSGVPERFSGSSSGTTVTLTISGAQAEDEADYYCQSVDNSGFYQVFGGGTQLTVLSGSSSSLEGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGLNDIFEAQKIEWHESRHHHHHH

SEQ ID NO: 51 - protein, 505 aa, Artificial Sequence
  Clone 10 scFv-Fc
QVQLQQSGPVLVKPSQTLSLTCAISGDSVSSDTTAWSWIRQSPSRGLEWLGWTYYRSKWFNYYAVSVKSRVTINPDTSKNQFSLQLSSVTPEDTAVYYCARGNGNGGMDVWGQGTTVTVSSGILGSGGGGSGGGGSGGGGSSYVLTQPPSVSVSPGQTARITCSGDALPKQYAYWYQQKPGQAPVLVIFKDSERPSGIPERFSGSSSGTTVTLTISGVQAEDEADYYCQSADNSGFYQVFGGGTQLTVLSGSSSSLEGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGLNDIFEAQKIEWHESRHHHHHH

SEQ ID NO: 52 - protein, 514 aa, Artificial Sequence
  Clone 22 scFv-Fc
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGSYGSGLVWGQGTTVTVSSGSASAPTGILGSGGGGSGGGGSGGGGSNFMLTQPHSVSESPGKTVTISCTGSSGSIASNYVQWYQQRPGSAPTTVIYEDDQRPSGVPDRFSGSLDTSSNSASLTISGLRPEDEADYYCQSYDNKDNVVFGGGTKVTVLSGSSSSLEGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGLNDIFEAQKIEWHESRHHHHHH

SEQ ID NO: 53 - protein, 506 aa, Artificial Sequence
  Clone 30 scFv-Fc
EVQLLETGGGLVQPGGSLRLSCAASGFTFSSSAMHWVRQAPGKGLEWVAAISYDGSDTYYADSVKGRFTISRDNAKNTLYLQMNSLRDEDTAVYYCARDQVLGSGSLDYWGQGTLVTVSSGILGSGGGGSGGGGSGGGGSNFMLTQPHSVSESPGKTVTISCTRSSGSIGSYYVQWYQQRPGSPPTTVIYEDDKRPSGVPDRFSGSIDSSSNSASLTISGLKTEDEADYYCQSYDSSHVVFGGGTKLTVLSGSSSSLEGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGLNDIFEAQKIEWHESRHHHHHH

SEQ ID NO: 54 - protein, 30 aa, Artificial Sequence
  Heavy chain framework region 1
QVQLQQSGPVLVKPGQTLSLTCAISGDSIS

SEQ ID NO: 55 - protein, 185 aa, Homo sapiens
MARGAALALLLFGLLGVLVAAPDGGFDLSDALPDNENKKPTAIPKKPSAGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

SEQ ID NO: 56 - protein, 14 aa, Artificial Sequence
  Rhesus macaque CD99, residues 63-76
GNDDPPPPNPPKPK

SEQ ID NO: 57 - protein, 14 aa, Artificial Sequence
  Mus musculus CD99, residues 74-87
EKPGNRPQPDPKPP

SEQ ID NO: 58 - protein, 14 aa, Artificial Sequence
  Homo sapiens Glycoprotein Xg, residues 63-76
GNIYPRPKPRPQPQ

SEQ ID NO: 59 - protein, 14 aa, Artificial Sequence
  Homo sapiens CD99L2, residues 130-143
DRDDGRRKPIAGGG

SEQ ID NO: 60 - protein, 14 aa, Artificial Sequence
  Matrix metalloproteinase 15, residues 328-341
DHRPPRPPQPPPPG

SEQ ID NO: 61 - protein, 13 aa, Artificial Sequence
  RIM 2, residues 518-531
QSESVRPPPPKPH

SEQ ID NO: 62 - protein, 14 aa, Artificial Sequence
  Adhesion G-protein coupled receptor V1, residues 3905-3918
ENDDPRGIFMFHVT

SEQ ID NO: 63 - protein, 14 aa, Artificial Sequence
  Nodal homolog isoform 2, residues 178-191
GECWPRPPTPPATN

SEQ ID NO: 64 - protein, 14 aa, Artificial Sequence
  Anoctamin-8, residues 1135-1148
PMPLPRPPTPPAGC

SEQ ID NO: 65 - protein, 14 aa, Artificial Sequence
  Anoctamin-8, residues 1123-1136
RTRRSRSPAPPPPM

SEQ ID NO: 66 - protein, 14 aa, Artificial Sequence
  FGFRL1, residues 355-368
VLPDPKPPGPPVAS

SEQ ID NO: 67 - protein, 13 aa, Artificial Sequence
  Fibrillin 2, residues 29-41
QPQPPPPKPPRPQ

SEQ ID NO: 68 - protein, 14 aa, Artificial Sequence
  Psoriasis susceptibility 1 candidate, residues 85-98
EPPRTDPPQPPRPD

SEQ ID NO: 69 - protein, 14 aa, Artificial Sequence
  Sciellin, residues 182-195
REPGVHPPIPPKPS

SEQ ID NO: 70 - protein, 14 aa, Artificial Sequence
  Sciellin, residues 204-217
LRQDNRQIHPPKPG

SEQ ID NO: 71 - protein, 14 aa, Artificial Sequence
  Anoctamin-8, residues 1194-1207
FYSLPPPPLPPTSD

SEQ ID NO: 72 - protein, 14 aa, Artificial Sequence
  Mucolipin-1, residues 195-208
VDPPERPPPPPSDD

SEQ ID NO: 73 - protein, 14 aa, Artificial Sequence
  Fibrillin  2, residues 34-47
PPKPPRPQPPPQQV

SEQ ID NO: 74 - protein, 14 aa, Artificial Sequence
  Psoriasis susceptability 1 candidate, residues 49-62
VPGDPWPGAPPLFE

SEQ ID NO: 75 - protein, 14 aa, Artificial Sequence
  Psoriasis susceptability 1 candidate, residues 77-90
PETGVWPPEPPRTD

SEQ ID NO: 76 - protein, 14 aa, Artificial Sequence
  Sciellin, residues 159-172
KRQSWFPPPPPGYN

SEQ ID NO: 77 - protein, 6 aa, Artificial Sequence
  2VWF
IQPPVN

SEQ ID NO: 78 - protein, 6 aa, Artificial Sequence
  2W0Z
PVPPRR

SEQ ID NO: 79 - protein, 9 aa, Artificial Sequence
  1AZE
APPPRPPKP

SEQ ID NO: 80 - protein, 8 aa, Artificial Sequence
  2HO2
PPPPPPPL

SEQ ID NO: 81 - protein, 11 aa, Artificial Sequence
  1EG4
TPYRSPPPYVP

SEQ ID NO: 82 - protein, 10 aa, Artificial Sequence
  1L2Z
SHRPPPPGHR

SEQ ID NO: 83 - protein, 15 aa, Artificial Sequence
  Clone 10A1 epitope
GENDDPRPPNPPKPM

(19) United States
(12) Patent Application Publication
(10) Pub. No.: US 2024/0190985 A1
(43) Pub. Date: Jun. 13, 2024

(54) THERAPEUTIC CD99 ANTIBODIES

(71) Applicant: NEW YORK UNIVERSITY, New York, NY (US)

(72) Inventors: Shohei KOIDE, New York, NY (US); Christopher P. PARK, New York, NY (US); Larizbeth A. ROMERO, Yonkers, NY (US); Takamitsu HATTORI, West New York, NJ (US)

(21) Appl. No.: 18/555,353

(22) PCT Filed: Apr. 15, 2022
(86) PCT No.: PCT/US2022/024998

§ 371 Date: Oct. 13, 2023

Related U.S. Application Data
(60) Provisional application No. 63/175,131, filed on Apr. 15, 2021.
(60) Provisional application No. 63/291,232, filed on Dec. 17, 2021.

(51) Int. Cl. C07K 16/28; A61K 39/00; A61P 35/02

(52) U.S. Cl. CPC C07K 16/2896; A61P 35/02; A61K 2039/505; C07K 2317/24; C07K 2317/31; C07K 2317/52; C07K 2317/565; C07K 2317/622; C07K 2317/73

(57) ABSTRACT

The present disclosure relates to an antibody-based molecule that binds CD99. This antibody-based molecule comprises a heavy chain variable region having: (i) a complementarity—determining region 1 (CDR-H1) comprising an amino acid sequence of any one of SEQ ID NOs: 1-3, or a modified amino acid sequence of any one of SEQ ID NOs: 1-3; (ii) a complementarity—determining region 2 (CDR-H2) comprising an amino acid sequence of any one of SEQ ID NOs: 4-10, or a modified amino acid sequence of any one of SEQ ID NOs: 4-10; and/or (iii) a complementarity—determining region 3 (CDR-H3) comprising an amino acid sequence of any one of SEQ ID NOs: 11-14, or a modified amino acid sequence of any one of SEQ ID NO: 11-14. Also disclosed are isolated polynucleotides encoding the antibody-based molecules, vectors comprising said isolated polynucleotides, as well as pharmaceutical compositions comprising the same and methods of use thereof.

This application claims the priority benefit of U.S. Provisional Patent Application Ser. No. 63/175,131, filed Apr. 15, 2021, and U.S. Provisional Patent Application 63/291,232, filed Dec. 17, 2021, each of which is hereby incorporated by reference in its entirety.

This invention was made with government support under Grant Nos. R01 CA194864, R01 CA212608 and R21 AI158997 awarded by the National Institutes of Health. The government has certain rights in this invention.

### FIELD

The present disclosure relates to antibody-based molecules, including full-length antibodies, epitope-binding domains thereof, and antibody derivatives. The present disclosure further discloses methods of treating CD99-expressing cancer in a subject.

### BACKGROUND

T-cell acute lymphoblastic leukemia (T-ALL) is a devastating disease in both children and adults, and current therapies come with a high risk of treatment related toxicity (Hurwitz et al., “Substituting Dexamethasone for Prednisone Complicates Remission Induction in Children with Acute Lymphoblastic Leukemia,” Cancer 88:1964-1969 (2000) and Real et al., “Gamma-Secretase Inhibitors Reverse Glucocorticoid Resistance in T Cell Acute Lymphoblastic Leukemia,” Nat. Med. 15:50-58 (2009)). T-ALL originates in T-lymphocyte progenitors and is characterized by the accumulation of leukemia cells in the bone marrow and reduced capacity to produce mature hematopoietic cells (Pui & Jeha, “New Therapeutic Strategies for the Treatment of Acute Lymphoblastic Leukaemia,” Nat. Rev. Drug Discov. 6:149-165 (2007)). Current therapies for T-ALL include chemotherapy, radiation, targeted therapy, and stem cell transplant (Pui & Jeha, “New Therapeutic Strategies for the Treatment of Acute Lymphoblastic Leukaemia,” Nat. Rev. Drug Discov. 6:149-165 (2007)). Current treatment options have dramatically improved patients' prognoses, with pediatric T-ALL patients showing a 75% overall survival rate (Goldberg et al., “Childhood T-Cell Acute Lymphoblastic Leukemia: The Dana-Farber Cancer Institute Acute Lymphoblastic Leukemia Consortium Experience,” J. Clin. Oncol. 21:3616-3622 (2003)). However, about 20% of these patients relapse (Chessells et al., “Long-Term Follow-up of Relapsed Childhood Acute Lymphoblastic Leukaemia,” Br. J. Haematol. 123:396-405 (2003)). Adult patients show poorer response to T-ALL therapies, with a complete remission rate of about 30% for primary relapsed or refractory T-ALL (Fielding et al., “Outcome of 609 Adults after Relapse of Acute Lymphoblastic Leukemia (ALL); an MRC UKALL12/ECOG 2993 Study,” Blood 109:944-950 (2007); Marks et al., “T-Cell Acute Lymphoblastic Leukemia in Adults: Clinical Features, Immunophenotype, Cytogenetics, and Outcome from the Large Randomized Prospective Trial (UKALL XII/ECOG 2993),” Blood 114:5136-5145 (2009); and Thomas et al., “Primary Refractory and Relapsed Adult Acute Lymphoblastic Leukemia: Characteristics, Treatment Results, and Prognosis with Salvage Therapy,” Cancer 86:1216-1230 (1999)). T-ALL relapse can be attributed to mutations that activate different oncogenic pathways, which in turn cause patients to have diminished responses to therapies (Liu et al., “The Genomic Landscape of Pediatric and Young Adult T-Lineage Acute Lymphoblastic Leukemia. Nat Genet 49:1211-1218 (2017); Szczepanski et al., “Late Recurrence of Childhood T-Cell Acute Lymphoblastic Leukemia Frequently Represents a Second Leukemia Rather Than a Relapse: First Evidence for Genetic Predisposition,” J. Clin. Oncol. 29:1643-1649 (2011); and Yadav et al., “Heterogeneity in Mechanisms of Emergent Resistance in Pediatric T-Cell Acute Lymphoblastic Leukemia,” Oncotarget. 7:58728-42 (2016)). New treatment options that are safer and more effective in all T-ALL patients, regardless of the underlying driving mutations, are necessary to overcome this disease.

CD99 has been proposed as a therapeutic target in T-ALL because it is overexpressed in leukemia cells relative to normal cells in nearly all T-ALLs (Darling et al., “Cloning an Expressed Gene Shared by the Human Sex Chromosomes,” Proc Natl Acad Sci USA 83:135-139 (1986); Husak et al., “Death Induction by CD99 Ligation in TEL/AML1-Positive Acute 20 Lymphoblastic Leukemia and Normal B Cell Precursors. J. Leukoc. Biol. 88:405-412 (2010); Levy et. al., “A Human Thymus-Leukemia Antigen Defined by Hybridoma Monoclonal Antibodies. Proc Natl Acad Sci USA 76:6552-6556 (1979); and Pettersen et al., “CD99 Signals Caspase-Independent T Cell Death,” J. Immunol. 166:4931-4942 (2001)) and is overexpressed at both diagnosis and relapse (Cox et al., “Investigating CD99 Expression in Leukemia Propagating Cells in Childhood T Cell Acute Lymphoblastic Leukemia,” PLoS One 11:e0165210 (2016)). The CD99 antigen is highly expressed on the surface of T-ALL cells with cell-surface CD99 levels about seven times higher in T-ALL blasts compared with normal T cells, and thus this protein can be used as a marker to assess minimal residual disease (Dworzak et al., “CD99 Expression in T-Lineage ALL: Implications for Flow Cytometric Detection of Minimal Residual Disease,” Leukemia 18, 703-708 (2004)). CD99 is also overexpressed in other leukemia types, including acute myeloid leukemia (AML) (Chung et al., “CD99 is a Therapeutic Target on Disease Stem Cells in Myeloid Malignancies,” Sci. Transl. Med. 9(374):eaaj2025 (2017)) and Ewing sarcoma (Ambros et al., “MIC2 is a Specific Marker for Ewing's Sarcoma and Peripheral Primitive Neuroectodermal Tumors. Evidence for a Common Histogenesis of Ewing's Sarcoma and Peripheral Primitive Neuroectodermal Tumors from MIC2 Expression and Specific Chromosome Aberration,” Cancer 67:1886-1893 (1991)). It is expressed at significantly lower levels on the surface of normal cells including endothelial cells in the blood vessels (Ambros et al., “MIC2 is a Specific Marker for Ewing's Sarcoma and Peripheral Primitive Neuroectodermal Tumors. Evidence for a Common Histogenesis of Ewing's Sarcoma and Peripheral Primitive Neuroectodermal Tumors from MIC2 Expression and Specific Chromosome Aberration,” Cancer 67:1886-1893 (1991)), which should afford a therapeutic window for anti-CD99 directed leukemia and cancer therapies.

Previous studies demonstrated that a mouse anti-CD99 IgM antibody has a cytotoxic effect both in vitro and in mouse models of AML (Chung et al., “CD99 is a Therapeutic Target on Disease Stem Cells in Myeloid Malignancies,” Sci. Transl. Med. 9(374):eaaj2025 (2017)), as well as in vitro models of T-ALL (Pettersen et al., “CD99 Signals Caspase-Independent T Cell Death,” J. Immunol. 166:4931-4942 (2001)). Although these studies demonstrated that CD99 is a promising target in T-ALL, it was not clear whether specific molecular features of the mouse IgM antibody were essential for its cytotoxic effect. Given the challenges associated with producing IgM antibodies (Clinical Trial NCT01123304: “A Phase 1 Clinical Trial of Intra-Tumoral Injection of Human IgM Monoclonal Antibody (MORAb-028) to the Ganglioside GD2 in Subjects With Injectable Metastatic Melanoma”, which was terminated due to lack of availability of investigational product) and the necessity of humanization, one might wish to generate therapeutic antibodies in a format that has been successfully tested in a clinical setting, such as IgG and its derivatives.

The present invention is directed to overcoming these and other deficiencies in the art.

### SUMMARY

A first aspect of the disclosure relates to an antibody-based molecule that binds CD99. This antibody-based molecule comprises a heavy chain variable region having:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

A second aspect of the disclosure relates to an antibody-based molecule that binds CD99. This antibody-based molecule comprises one or more amino acid modifications to a heavy chain framework region that enhances stability of the antibody-based molecule that binds CD99, where the heavy chain framework region comprises the partial amino acid sequence of: QVQLQQSGPVLVKPGQTLSLTCAISGDSIS (SEQ ID NO: 54), and further comprises:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

A third aspect of the disclosure relates to an antibody-based molecule that binds CD99 according to the first and/or second aspects, which antibody-based molecule further includes a light chain variable region that comprises:
(i) a complementarity-determining region 1 (CDR-L1) having an amino acid sequence of any one of SEQ ID NOs: 15-18, or a modified amino acid sequence of any one of SEQ ID NO: 15-18, said modified sequence having at least 80% sequence identity to any one of SEQ ID NO: 15-18;
(ii) a complementarity-determining region 2 (CDR-L2) having an amino acid sequence of any one of SEQ ID NOs: 19-22, or a modified amino acid sequence of any one of SEQ ID NO: 19-22, said modified sequence having at least 80% sequence identity to any one of SEQ ID NO: 19-22; and/or
(iii) a complementarity-determining region 3 (CDR-L3) having an amino acid sequence of any one of SEQ ID NOs: 23-27, or a modified amino acid sequence of any one of SEQ ID NO: 23-27, said modified sequence having at least 80% sequence identity to any one of SEQ ID NO: 23-27.

A fourth aspect of the disclosure relate to isolated polynucleotides encoding an antibody-based molecule according to the present disclosure.

A fifth aspect of the disclosure relates to a vector comprising an isolated polynucleotide according to the present disclosure.

A sixth aspect of the disclosure relates to a host cell comprising a vector according to the present disclosure.

A seventh aspect of the disclosure relates to a pharmaceutical composition comprising: an antibody-based molecule, a polynucleotide, or a vector according to the present disclosure, and a pharmaceutically acceptable carrier.

An eighth aspect of the disclosure relates to a method of inducing apoptosis in a population of CD99-expressing cancer cells. This method involves contacting a population of CD99-expressing cancer cells with a therapeutically effective amount of a CD99 antibody-based molecule as described herein, an isolated polynucleotide encoding the antibody-based molecule as described herein, a vector comprising the polynucleotide as described herein, or a pharmaceutical composition as described herein, in an amount effective to induce apoptosis in the population of CD99-expressing cancer cells.

A ninth aspect of the disclosure relates to a method of treating a CD99-expressing cancer in a subject. This method involves administering, to a subject having a CD99-expressing cancer, an antibody-based molecule as described herein, an isolated polynucleotide encoding the antibody-based molecule as described herein, a vector comprising the polynucleotide as described herein, or a pharmaceutical composition as described herein, in an amount effective to treat the cancer in the subject.

The examples of the present disclosure describe the development of a human anti-CD99 antibody and the demonstration that the cytotoxic effect requires an antibody valency of three or more. Based on this mechanistic insight, an IgG-based human antibody with a valency of four that shows cytotoxic efficacy selective to T-ALL cells was developed. The crystal structure of an antibody-antigen complex rationalizes the antibody specificity and provides a guide for further development.

### BRIEF DESCRIPTION OF THE DRAWINGS

FIGS. 1A-G illustrate the generation of human anti-CD99 antibodies. FIG. 1A illustrates the binding titrations of three antibody clones in the scFv-Fc format to purified SUMO-CD99 fusion protein and SUMO protein (a negative control) immobilized on beads. The apparent K_{D} values are from curve fitting of a 1:1 binding model. Values indicated are the means and standard deviations from curve fitting (n=3). K_{D} values are expressed in terms of nM Fv to minimize ambiguity across different antibody formats. FIGS. 1B-1C illustrate the binding of the antibodies to human CD99 and close homologs in human using 600 nM Fv of scFv-Fc (FIG. 1B) and to CD99 from different species using 1 μM Fv of scFv-Fc (FIG. 1C), n=3. FIGS. 1D-lE illustrate flow cytometry analysis of antibody clones following staining of a T-ALL cell line that expresses CD99 (Jurkat, left) and cells that do not express CD99 (ExpiCHO, right). In FIG. 1D, flow cytometry detection of CD99 on Jurkat and ExpiCHO cellswas carried out using a commercial mouse anti-CD99 antibody, HO36-1.1. The black dashed lines mark thresholds defined with the data with only the secondary antibody. ExpiCHO cells serve as a negative control. FIG. 1E illustrates flow cytometry detection of CD99 on cells using clones 10, 22, and 30. The black dashed lines mark thresholds defined with detection using mouse Fc, equivalent to an isotype control. FIG. 1F illustrates binding titration curves of clone 10 (top) and affinity matured clone 10A1 (bottom) in the Fab format to purified SUMO-CD99 fusion protein immobilized on beads (n=3). FIG. 1G illustrates binding titration curves of clone 10 (top) and 10A1 (bottom) in the scFv-Fc format to endogenously expressed CD99 on Jurkat cells (n=3). The data for nonbinding mouse Fc are shown as open circles. The 10 or 10A1 antibodies are shown as filled circles. The apparent K_{D} values are from curve fitting of a 1:1 binding model. Values indicated are the means and standard deviations (n=3).

FIGS. 2A-C illustrate epitope mapping of human anti-CD99 antibodies using truncated forms of CD99 and Western blot detection. FIG. 2A is a schematic map depicting truncated CD99 proteins used (spanning residues 23-11 of SEQ ID NO: 55) and deduced epitopes. Group A (two black bars at the top and two black bars at the bottom) denotes CD99 fragments that were not bound by any of the antibodies; Group B (three red bars) denotes fragments bound by clones 22 and 30; Group C (single green bar) denotes a fragment bound by clones 22 and 30 and at a lower degree to HO36-1.1 and clone 10; Group D (three purple bars) denotes fragments bound by clones 10, 22, and 30 and HO36-1.1; Group E (single brown bar) denotes a fragment bound by clone 10 and HO36-1.1 and to a lesser degree by clone 30; and Group F (two blue bars) denotes fragments bound by clone 10 and HO36-1.1. The locations of the deduced epitopes are shaded. FIGS. 2B-C illustrates Western blotting data. Anti-His antibody (bottom) was used to confirm sample loading. NegGFP denotes negative control GFP.

FIGS. 3A-I illustrate the cytotoxic effect and binding characteristics of the anti-CD99 antibody, 10A1, in different formats. In FIG. 3A, dose-dependent cytotoxic effects on KOPT-K1, as measured by Annexin V staining after an 18-hour treatment are shown. From left to right: Fab, IgG-LALA, IgG-HCscFv, IgG-E345R, and IgG-RGY. The antibody formats are schematically shown. Data for the nonbinding isotype controls are shown as open circles, and those for the 10A1 antibodies are shown as filled circles (n=3). In FIG. 3B, binding titration curves of 10A1 antibodies to endogenously expressed CD99 on KOPT-K1 cells are shown. From left to right: Fab, IgG-LALA, IgG-HCscFv, IgG-E345R, and IgG-RGY. Data for the nonbinding isotype controls are shown as open circles, and those for the 10A1 antibodies are shown as filled circles (n=3). In FIG. 3C, cell surface levels of CD99 of healthy donor PBMC, T-ALL samples, and the KOPT-K1 cell line are shown. In FIG. 3D, cytotoxic effects of 18-hour treatment with 2.0 μM Fv (equivalent to 500 nM molecule) 10A1 HCscFv on healthy control PBMCs, and leukemia cells derived from T-ALL patients, and KOPT-K1 cells (n=3) are shown. In FIG. 3E, cell surface levels of CD99 of healthy donor PBMC samples, and KOPT-K1 cell line are shown. In FIG. 3F, cytotoxic effects of 18-hour treatment with 2.0 μM Fv 10A1 HCscFv on healthy control PBMC samples and KOPT-K1 cells (n=3) are shown. In FIG. 3G, cell surface levels of CD99 on OCI-AML3 (AML cell line), CCRF-SB (B-ALL cell line), and MOLT3 and KOPT-K1 (T-ALL cell lines) are shown. In FIG. 3H, cytotoxic effects of 2.0 μM Fv 10A1 HCscFv on T-ALL and B-ALL cell lines after 18 hours are shown. In FIG. 3I, cytotoxic effects of 2.0 μM Fv 10A1 RGY on AML and T-ALL cell lines after 18 hours are shown. P values were calculated using 2-way ANOVA with Tukey's multiple comparisons test.

FIGS. 4A-C demonstrate that a valency of three is required and sufficient for inducing KOPT-K1 cytotoxicity. FIG. 4A is a schematic depiction of bi, tri, and tetravalent Streptavidin (SAV)-Fab complexes. “A” represents wildtype SAV with ‘alive’ biotin-binding site; and “D” represents a mutant SAV with ‘dead’ biotin-binding site. A dead biotin binding site is shown as a blue circle (with no Fab fragment attached), and an active biotin binding site as a red circle (with Fab fragment attached). FIG. 4B shows cytotoxicity, as measured by Annexin V staining, of bi-, tri-, and tetravalent SAV-Fab complexes (400 nM Fv) in KOPT-K1 cells treated for 18 hours (n=3). P values were calculated using ordinary 1-way ANOVA with Tukey's multiple comparisons test. FIG. 4C shows binding titration curves of bivalent, trivalent, and tetravalent SAV-Fab complexes to endogenously expressed CD99 on KOPT-K1 cells (n=3).

FIGS. 5A-D illustrate the crystal structure of the 10A1 Fab in complex with a CD99 peptide, PDB ID: 7SFX, which is hereby incorporated by reference in its entirety. FIG. 5A is a cartoon depiction of the complex. FIG. 5B illustrates an overlay of proline-rich peptides from CD99 residues 70-76 of SEQ ID NO: 55 (magenta & purple), SH3 domain binders: 2VWF (salmon, chain B: IQPPVN, SEQ ID NO: 77), 1AZE (red, chain B: PVPPRR, SEQ ID NO: 78), and 2W0Z (brown, chain B: APPPRPPKP, SEQ ID NO: 79); WW-domain binders: 2HO2 (light blue, chain B: PPPPPPPL, SEQ ID NO: 80) and 1EG4 (blue, chain A: TPYRSPPPYVP, SEQ ID NO: 81); and the proline-rich tail of CD2: 1L2Z (orange, chain B: SHRPPPPGHR, SEQ ID NO: 82). FIG. 5C illustrates a surface representation of 10A1 Fab showing two distinct binding pockets (enclosed circles) for the CD99 peptide residues 70-76 of SEQ ID NO: 54. FIG. 5D shows the 10A1/CD99 interface highlighting key residues in the 10A1 Fab. Residues in yellow (heavy chain residue Y54; light chain residues Q30, Y31, D51, D90, and F95) showed a substantial loss in CD99 binding upon mutation, as shown in FIG. 5E, whereas residues in gray (heavy chain residues K58, F60; light chain residues K29, K49, Y96, N105) tolerated mutations. FIG. 5E illustrates the effects of 10A1 fab mutagenesis studies, in the yeast display format. Binding signals with 100 nM SAV-CD99 complex are shown (n=3). SAV650, Streptavidin DyLight650.

FIG. 6 is a table summarizing cell-based assays utilizing antibodies and complexes that bind to CD99 highlighting: valency, homotypic cell aggregation, cytotoxicity, IC₅₀ values, and apparent K_{D} values.

FIG. 7 is table showing the partial alignment of mouse and monkey CD99 homologs, and BLAST results showing similarities with the clone 10 epitope. Shaded residues are identical to the corresponding residue in the human CD99 sequence (corresponding to residues 63-76 of SEQ ID NO: 55). The underlined portion of the human CD99 sequence is resolved in protein structure in FIGS. 5A-D.

### DETAILED DESCRIPTION

The present disclosure relates to antibody-based molecules, including antibodies, epitope-binding domains thereof, and antibody derivative as described herein, that are capable of binding CD99. Such antibody-based molecules are useful for the treatment of conditions where a subject is in need of treating CD99-expressing cancer in a subject.

Aspects of the present disclosure are directed to antibody-based molecules that bind an epitope of CD99. CD99 is a 32 kDa cell surface glycoprotein encoded by the MIC2 gene located in the pseudoautosomal region at the end of the short arms of the X and Y chromosomes. CD99 is involved in important physiological functions, including cellular adhesion, apoptosis, T cell and thymocyte differentiation, monocyte migration, and intracellular adhesion between lymphocytes and endothelial cells. In pathological conditions, and in particular in acute myeloid leukemia (AML), CD99 plays a key role in mediating prospective separation of leukemic stem cells (LSCs) from functionally normal hematopoietic stem cells (HSCs) (see, e.g., Chung et al., “CD99 is a Therapeutic Target on Disease Stem Cells in Myeloid Malignancies,” Sci. Transl. Med. 9(374): eaaj2025 (2017), which is hereby incorporated by reference in its entirety). Monoclonal antibodies (mAbs) targeting CD99 induce the death of AML and MDS cells in a SRC-family kinase dependent manner in the absence of immune effector cells or complement, 10 and administration of anti-CD99 mAbs exhibit anti-leukemic activity in AML xenografts. Accordingly, CD99 is a marker of AML and myelodysplastic syndrome (MDS) stem cells, as well as a promising therapeutic target in these disorders.

High CD99 expression has been observed in Ewing sarcoma (EWS), lymphoblastic lymphoma/leukemia, myeloid malignancies, and malignant gliomas, as well as sporadically in synovial sarcoma, mesenchymal chondrosarcoma, rhabdomyosarcoma, thymic tumors, hemangiopericytoma, gastrointestinal and pulmonary neuroendocrine tumors, sex-cord stromal tumors, and a small percentage of breast carcinomas (see, e.g., Pasello et al., “CD99 at the Crossroads of Physiology and Pathology,” J. Cell Commun. Signal. 12(1):55-68 (2018), which is hereby incorporated by reference in its entirety). CD99 has been reported to have a marked effect on the migration, invasion, and metastasis of tumor cells through multiple mechanisms of action, thereby emerging as a therapeutic target in CD99-expressing cancers.

High CD99 expression has also been observed in T cell acute lymphoblastic leukemia (T-ALL) (Darling S M et al. “Cloning an Expressed Gene Shared by the Human Sex Chromosomes,” Proc. Natl. Acad. Sci. USA 83(1):135-139 (1986), and Levy R et al. “A Human Thymus-Leukemia Antigen Defined by Hybridoma Monoclonal Antibodies,” Proc. Natl. Acad. Sci. USA 76(12):6552-6556 (1979), which are hereby incorporated by reference in their entirety). CD99 levels are about seven times higher in T-ALL blasts compared to normal T cells, and this protein can be used as a marker to assess minimal residual disease (Dworzak M N et al., “CD99 Expression in T-Lineage ALL: Implications for Flow Cytometric Detection of Minimal Residual Disease,” Leukemia 18(4):703-708 (2004), which is hereby incorporated by reference in its entirety). In T-ALL CD99 is overexpressed at both diagnosis and relapse (Cox et al., “Investigating CD99 Expression in Leukemia Propagating Cells in Childhood T Cell Acute Lymphoblastic Leukemia,” PloS one 11(10):e0165210 (2016), which is hereby incorporated by reference in its entirety). Targeting CD99 using mAbs induces cell death in T-ALL cells (Pettersen R D et al., “CD99 Signals Caspase-Independent T Cell Death,” J. Immunol. 166(8):4931-42 (2001), which is hereby incorporated by reference in its entirety), making CD99 an attractive therapeutic target in this disease.

The amino acid sequence of human CD99 has the amino acid sequence of SEQ ID NO: 55 below.

MARGAALALLLFGLLGVLVAAPDGGFDLSDALPDNENKKPTAIPKKPSAG
DDFDLGDAVVDGENDDPRPPNPPKPMPNPNPNHPSSSGSFSDADLADGVS
GGEGKGGSDGGGSHRKEGEEADAPGVIPGIVGAVVVAVAGAISSFIAYQK
KKLCFKENAEQGEVDMESHRNANAEPAVQRTLLEK

See NCBI Ref. No. NP_002405.1, which is hereby incorporated by reference in its entirety.

In some embodiments, the CD99 antibodies described herein bind to amino acid residues 33-52 of SEQ ID NO: 55, amino acid residues 43-52 of SEQ ID NO: 55, amino acid residues 63-76 of SEQ ID NO: 55, or amino acid residues 63-74 of SEQ ID NO: 55.

Antibody-based molecules include, without limitation, full antibodies, epitope binding fragments of whole antibodies, and antibody derivatives. An epitope binding fragment of an antibody can be obtained through the actual fragmenting of a parental antibody, for example, a Fab or (Fab)₂ fragment. Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

An antibody of the present invention is an intact immunoglobulin as well as a molecule having an epitope-binding fragment thereof. As used herein, the terms “fragment”, “region”, and “domain” are generally intended to be synonymous, unless the context of their use indicates otherwise. Naturally occurring antibodies typically comprise a tetramer which is usually composed of at least two heavy (H) chains and at least two light (L) chains. Each heavy chain is comprised of a heavy chain variable (V_{H}) region and a heavy chain constant (C_{H}) region, usually comprised of three domains (C_{H}1, C_{H}2 and C_{H}3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (V_{L}) region and a light chain constant (C_{L}) region. Light chains include kappa chains and lambda chains. The heavy and light chain variable regions are typically responsible for antigen recognition, while the heavy and light chain constant regions may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed “complementarity determining regions,” or “CDRs,” that are interspersed with regions of more conserved sequence, termed “framework regions” (FR). Each V_{H} and V_{L} region is composed of three CDR domains and four FR domains arranged from amino-terminus to carboxy-terminus in the following order: FR1-CDR1-FR2-CDR2-FR3-CDR3-FR4. The variable regions of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally-occurring antibody in amino acid sequence.

Fragments of antibodies (including Fab and (Fab)₂ fragments) that exhibit epitope-binding ability can be obtained, for example, by protease cleavage of intact antibodies. Single domain antibody fragments possess only one variable domain (e.g., V_{L} or V_{H}). Examples of the epitope-binding fragments encompassed within the present invention include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain; (v) dAb fragments (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a V_{H} or V_{L} domain and are also called domain antibodies (Holt et al. “Domain Antibodies: Proteins For Therapy,” Trends Biotechnol. 21(11):484-490 (2003), which is hereby incorporated by reference in its entirety); (vi) camelid or nanobodies (Revets et al. “Nanobodies As Novel Agents For Cancer Therapy,” Expert Opin. Biol. Ther. 5(1):111-124 (2005), which is hereby incorporated by reference in its entirety), and (vii) isolated complementarity determining regions (CDR). An epitope-binding fragment may contain 1, 2, 3, 4, 5 or all 6 of the CDR domains of such antibody.

Such antibody fragments are obtained using conventional techniques known to those of skill in the art. For example, F(ab′)₂ fragments may be generated by treating a full-length antibody with pepsin. The resulting F(ab′)₂ fragment may be treated to reduce disulfide bridges to produce Fab′ fragments. Fab fragments may be obtained by treating an IgG antibody with papain, and Fab′ fragments may be obtained with pepsin digestion of IgG antibody. A Fab′ fragment may be obtained by treating an F(ab′)₂ fragment with a reducing agent, such as dithiothreitol. Antibody fragments may also be generated by expression of nucleic acids encoding such fragments in recombinant cells (see, e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′)₂ fragment could include DNA sequences encoding the C_{H}1 domain and hinge region of the heavy chain, followed by a translational stop codon to yield such a truncated antibody fragment molecule. Suitable fragments capable of binding to a desired epitope may be readily screened for utility in the same manner as an intact antibody.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region, which are encoded by separate genes. Such gene sequences or their encoding cDNA are joined, using recombinant methods, by a flexible linker (typically encoding about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. USA 85:5879-5883 (1988), which are hereby incorporated by reference in their entirety). Alternatively, by employing a flexible linker that is not too short (e.g., less than about 9 residues) to enable the V_{L} and V_{H} regions of a different single polypeptide chains to associate together, one can form a bispecific antibody, having binding specificity for two different epitopes.

In another embodiment, the antibody derivative is a divalent or bivalent single-chain variable fragment, engineered by linking two scFvs together either in tandem (i.e., tandem scFv), or such that they dimerize to form diabodies (Holliger et al. “‘Diabodies’: Small Bivalent And Bispecific Antibody Fragments,” Proc. Natl. Acad. Sci. USA 90(14), 6444-8 (1993), which is hereby incorporated by reference in its entirety). In yet another embodiment, the antibody is a trivalent single chain variable fragment, engineered by linking three scFvs together, either in tandem or in a trimer formation to form triabodies. In another embodiment, the antibody is a tetrabody single chain variable fragment. In another embodiment, the antibody is a “linear antibody” which is an antibody comprising a pair of tandem Fd segments (V_{H}—C_{H}1-V_{H}-C_{H}1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3).

These and other useful antibody fragments and derivative in the context of the present invention are discussed further herein. It also should be understood that the term antibody-based molecule, unless specified otherwise, also includes antibody-like polypeptides, such as chimeric antibodies and humanized antibodies, and antibody fragments retaining the ability to specifically bind to the antigen (epitope-binding fragments) provided by any known technique, such as enzymatic cleavage, peptide synthesis, and recombinant techniques.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Either of the human light chain constant regions, kappa or lambda, may be used. If desired, the class of a CD99 antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see Kabat et al., SEQUENCES OF PROTEINS OF IMMUNOLOGICAL INTEREST, National Institutes of Health Publication No. 91-3242 (1992); Chothia et al., “Canonical Structures For The Hypervariable Regions of Immunoglobulins,” J. Mol. Biol. 196:901-917 (1987), which are hereby incorporated by reference in their entirety), by molecular modeling and/or empirically, or as described in Gonzales et al., “SDR Grafting of a Murine Antibody Using Multiple Human Germline Templates to Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

Phage display technology and/or yeast display technology can be used to increase (or decrease) CDR affinity of the antibody-based molecules of the present invention. These technologies, referred to as affinity maturation technologies, employ mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering by Codon-Based Mutagenesis in a Filamentous Phage Vector System,” J. Immunology 149:3903-3913 (1992); Feldhaus et al., “Flow-Cytometric Isolation of Human Antibodies from a Nonimmune Saccharomyces cerevisiae Surface Display Library,” Nat. Biotechnol. 21:163-170 (2003) and Chao et al., “Isolating and Engineering Human Antibodies Using Yeast Surface Display,” Nat. Protoc. 1:755-768 (2006), which are hereby incorporated by reference in their entirety). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise in Vitro Affinity Maturation of Vitaxin, an Alphav Beta3-Specific Humanized mAb,” Proc. Natl. Acad. Sci. USA 95:6037-6042 (1998); Yelton et al., “Affinity Maturation of the BR96 Anti-Carcinoma Antibody by Codon-Based Mutagenesis,” J. Immunology 155:1994 (1995), which are hereby incorporated by reference in their entirety). CDR walking, which randomizes the light chain, may be used (see, Schier, R. et al., “Isolation of Picomolar Affinity Anti-c-erbB-2 Single-Chain Fv by Molecular Evolution of the Complementarity Determining Regions in the Center of the Antibody Binding Site,” J. Mol. Biol. 263:551-567 (1996), which is hereby incorporated by reference in its entirety).

Methods for accomplishing such affinity maturation that are suitable for affinity maturation of the CD99 antibody molecule disclosed herein are described, for example, in Krause et al., “An Insertion Mutation That Distorts Antibody Binding Site Architecture Enhances Function of a Human Antibody,” MBio. 2(1): e00345-10 (2011); Kuan et al., “Affinity-Matured Anti-Glycoprotein NMB Recombinant Immunotoxins Targeting Malignant Gliomas And Melanomas,” Int. J. Cancer 10.1002/ijc.25645 (2010); Hackel et al., “Stability and CDR Composition Biases Enrich Binder Functionality Landscapes,” J Mol. Biol. 401(1):84-96 (2010); Montgomery et al., “Affinity Maturation and Characterization of a Human Monoclonal Antibody Against HIV-1 gp41,” MAbs 1(5):462-474 (2009); Gustchina et al., “Affinity Maturation by Targeted Diversification of the CDR-H2 Loop of a Monoclonal Fab Derived from a Synthetic Naïve Human Antibody Library and Directed Against the Internal Trimeric Coiled-Coil of Gp41 Yields a Set of Fabs With Improved HIV-1 Neutralization Potency and Breadth,” Virology 393(1):112-119 (2009); Finlay et al., “Affinity Maturation of a Humanized Rat Antibody for Anti-RAGE Therapy: Comprehensive Mutagenesis Reveals a High Level of Mutational Plasticity Both Inside and Outside the Complementarity-Determining Regions,” J. Mol. Biol. 388(3):541-558 (2009); Bostrom et al., “Improving Antibody Binding Affinity and Specificity for Therapeutic Development,” Methods Mol. Biol. 525:353-376 (2009); Steidl et al., “In Vitro Affinity Maturation of Human GM-CSF Antibodies by Targeted CDR-Diversification,” Mol. Immunol. 46(1):135-144 (2008); and Barderas et al., “Affinity Maturation of Antibodies Assisted by in silico Modeling,” Proc. Natl. Acad. Sci. USA 105(26):9029-9034 (2008), which are hereby incorporated by reference in their entirety.

In one aspect of the present disclosure, the CD99-antibody based molecules as described herein comprise the amino acid sequence of any one, any two, any three, any four, any five, or any six CDRs as provided in Tables 1 and 2 herein.

In one aspect, the antibody-based molecule that binds to CD99 comprises a heavy chain variable region, where the heavy chain variable region comprises:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

In another aspect, the antibody-based molecule that binds to CD99 comprises one or more amino acid modifications to a heavy chain framework region that enhance stability of the antibody-based molecule that binds CD99, where the heavy chain framework region comprises the partial amino acid sequence of: QVQLQQSGPVLVKPGQTLSLTCAISGDSIS (SEQ ID NO: 54), and further comprises:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

In some embodiments of the antibody-based molecules according to the present disclosure, the antibody-based molecule that binds to CD99 comprises a heavy chain variable region, where the heavy chain variable region is selected from the group consisting of:
(i) a heavy chain variable region comprising the CDR-H1 of SEQ ID NO: 1, the CDR-H2 of SEQ ID NO: 4, and the CDR-H3 of SEQ ID NO: 11 (corresponding to clone 10A1);
(ii) a heavy chain variable region comprising the CDR-H1 of SEQ ID NO: 1, the CDR-H2 of SEQ ID NO: 5, and the CDR-H3 of SEQ ID NO: 11 (corresponding to clone 10);
(iii) a heavy chain variable region comprising the CDR-H1 of SEQ ID NO: 2, the CDR-H2 of SEQ ID NO: 6, and the CDR-H3 of SEQ ID NO: 12 (corresponding to clone 22);
(iv) a heavy chain variable region comprising the CDR-H1 of SEQ ID NO: 3, the CDR-H2 of SEQ ID NO: 7, and the CDR-H3 of SEQ ID NO: 13 (corresponding to clone 30);
(v) a heavy chain variable region comprising the CDR-H1 of SEQ ID NO: 1, the CDR-H2 of SEQ ID NO: 8, and the CDR-H3 of SEQ ID NO: 11 (corresponding to clone 10A1 VH_T53I);
(vi) a heavy chain variable region comprising the CDR-H1 of SEQ ID NO: 1, the CDR-H2 of SEQ ID NO: 9, and the CDR-H3 of SEQ ID NO: 11 (corresponding to clone 10A1 VH_K58A);
(vii) a heavy chain variable region comprising the CDR-H1 of SEQ ID NO: 1, the CDR-H2 of SEQ ID NO: 10, and the CDR-H3 of SEQ ID NO: 11 (corresponding to clone 10A1 VH_F60A);
(viii) a heavy chain variable region comprising the CDR-H1 of SEQ ID NO: 1, the CDR-H2 of SEQ ID NO: 4, and the CDR-H3 of SEQ ID NO: 14 (corresponding to clone 10A1 VH_N105A); and/or
(ix) a heavy chain variable region comprising the CDR-H1 of SEQ ID NO: 1, the CDR-H2 of SEQ ID NO: 8, and the CDR-H3 of SEQ ID NO: 14 (corresponding to clone 10A1 VH_S15G/T53I/N105A).
   The sequences of the heavy chain CDR sequences are provided in Table 1 below.

TABLE 1
Heavy Chain CDR Sequences of CD99 Antibodies

| | CDR-H1 | CDR-H2 | CDR-H3 |
|---|---|---|---|
| | (Se- | (Se- | (Se- |
| mAb/Fab | quence & | quence & | quence & |
| clone | SEQ ID | SEQ ID | SEQ ID |
| name | NO) | NO) | NO) |
| 10A1 | SDTTAWS | WTYYRSKWFNYYAVSVKG | GNGNGGMDV |
| | 1 | 4 | 11 |
| 10 | SDTTAWS | WTYYRSKWFNYYAVSVKS | GNGNGGMDV |
| | 1 | 5 | 11 |
| 22 | SNSAAWN | RTYYRSKWYNDYAVSVKS | GSYGSGLV |
| | 2 | 6 | 12 |
| 30 | SSAMH | AISYDGSDTYYADSVKG | DQVLGSGSLDY |
| | 3 | 7 | 13 |
| 10A1 | SDTTAWS | WIYYRSKWFNYYAVSVKG | GNGNGGMDV |
| VH_ | 1 | 8 | 11 |
| T53I | | | |
| 10A1 | SDTTAWS | WTYYRSAWFNYYAVSVKG | GNGNGGMDV |
| VH_ | 1 | 9 | 11 |
| K58A | | | |
| 10A1 | SDTTAWS | WTYYRSKWANYYAVSVKG | GNGNGGMDV |
| VH_ | 1 | 10 | 11 |
| F60A | | | |
| 10A1 | SDTTAWS | WTYYRSKWFNYYAVSVKG | GNGAGGMDV |
| VH_ | 1 | 4 | 14 |
| N105A | | | |

In some embodiments, the heavy chain variable region of the antibody-based molecule further comprises human immunoglobulin heavy chain framework regions.

In some embodiments, the CD99 antibody-based molecules as disclosed herein further comprise a light chain variable region. The light chain variable region may comprise:
(i) a complementarity-determining region 1 (CDR-L1) having an amino acid sequence of any one of SEQ ID NOs: 15-18, or a modified amino acid sequence of any one of SEQ ID NO: 15-18, said modified sequence having at least 80% sequence identity to any one of SEQ ID NO: 15-18;
(ii) a complementarity-determining region 2 (CDR-L2) having an amino acid sequence of any one of SEQ ID NOs: 19-22, or a modified amino acid sequence of any one of SEQ ID NO: 19-22, said modified sequence having at least 80% sequence identity to any one of SEQ ID NO: 19-22; and/or
(iii) a complementarity-determining region 3 (CDR-L3) having an amino acid sequence of any one of SEQ ID NOs: 23-27, or a modified amino acid sequence of any one of SEQ ID NO: 23-27, said modified sequence having at least 80% sequence identity to any one of SEQ ID NO: 23-27.

In some embodiments, the light chain variable region of the CD99 antibody based molecule disclosed herein is selected from the group consisting of:
(i) a light chain variable region comprising the CDR-L1 of SEQ ID NO: 15, the CDR-L2 of SEQ ID NO: 19, and the CDR-L3 of SEQ ID NO: 23;
(ii) a light chain variable region comprising the CDR-L1 of SEQ ID NO: 15, the CDR-L2 of SEQ ID NO: 19, and the CDR-L3 of SEQ ID NO: 24;
(iii) a light chain variable region comprising the CDR-L1 of SEQ ID NO: 16, the CDR-L2 of SEQ ID NO: 20, and the CDR-L3 of SEQ ID NO: 25;
(iv) a light chain variable region comprising the CDR-L1 of SEQ ID NO: 17, the CDR-L2 of SEQ ID NO: 21, and the CDR-L3 of SEQ ID NO: 26;
(v) a light chain variable region comprising the CDR-L1 of SEQ ID NO: 18, the CDR-L2 of SEQ ID NO: 19, and the CDR-L3 of SEQ ID NO: 23;
(vi) a light chain variable region comprising the CDR-L1 of SEQ ID NO: 15, the CDR-L2 of SEQ ID NO: 22, and the CDR-L3 of SEQ ID NO: 23; and/or
(vii) a light chain variable region comprising the CDR-L1 of SEQ ID NO: 15, the CDR-L2 of SEQ ID NO: 19, and the CDR-L3 of SEQ ID NO: 27.
   The sequences of the light chain CDR sequences are provided in Table 2 below.

TABLE 2
Light Chain CDR Sequences of CD99 Antibodies

| | CDR-L1 | CDR-L2 | CDR-L3 |
|---|---|---|---|
| mAb/ | (Se- | (Se- | (Se- |
| Fab | quence & | quence & | quence & |
| clone | SEQ ID | SEQ ID | SEQ ID |
| name | NO) | NO) | NO) |
| 10A1 | SGDALPKQYAY | KDSERPS | QSVDNSGFYQV |
| | 15 | 19 | 23 |
| 10 | SGDALPKQYAY | KDSERPS | QSADNSGFYQV |
| | 15 | 19 | 24 |
| 22 | TGSSGSIASNYVQ | EDDQRPS | QSYDNKDNVV |
| | 16 | 20 | 25 |
| 30 | TRSSGSIGSYYVQ | EDDKRPS | QSYDSSHVV |
| | 17 | 21 | 26 |
| 10A1 | SGDALPAQYAY | KDSERPS | QSVDNSGFYQV |
| VL_ | 18 | 19 | 23 |
| K29A | | | |
| 10A1 | SGDALPKQYAY | ADSERPS | QSVDNSGFYQV |
| VL_ | 15 | 22 | 23 |
| K49A | | | |
| 10A1 | SGDALPKQYAY | KDSERPS | QSVDNSGFAQV |
| VL_ | 15 | 19 | 27 |
| Y96A | | | |

In some embodiments, the light chain variable region of the antibody-based molecule further comprises human immunoglobulin light chain framework regions.

Suitable amino acid modifications to the heavy chain CDR sequences and/or the light chain CDR sequences of the CD99 antibody-based molecule disclosed herein include, for example, conservative substitutions or functionally equivalent amino acid residue substitutions that result in variant CDR sequences having similar or enhanced binding characteristics to those of the CDR sequences disclosed herein as described above. Encompassed by the present disclosure are CDRs of Table 1 and 2 containing 1, 2, 3, 4, 5, or more amino acid substitutions (depending on the length of the CDR) that maintain or enhance CD99 binding of the antibody. The resulting modified CDRs are at least 70%, at least 75%, at least 80%, at least 85%, at least 90%, at least 95% similar in sequence to the CDRs of Tables 1 and 2. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain variable region CDRs of Table 1 and/or the light chain variable region CDRs of Table 2 may further comprise one or more internal neutral amino acid insertions or deletions that maintain or enhance CD99 binding.

In some embodiments, the antibody-based molecule that binds CD99 includes one or more amino acid modifications to a heavy chain and/or light chain framework region that enhance stability of the antibody-based molecule that binds CD99.

By way of example, and without limitation, the heavy chain framework region 1 contains the partial amino acid sequence of: QVQLQQSGPVLVKPGQTLSLTCAISGDSIS (SEQ ID NO: 54) or an amino acid sequence that shares at least 80%, 85%, 90%, or 95% identity thereto, and the heavy chain also contains one or more of the above-identified heavy chain CDR1, CDR2, and/or CDR3 regions set forth in Table 1.

One exemplary antibody-based molecule that binds CD99 includes a heavy chain that includes framework region 1 according to SEQ ID NO: 54 in combination with the CDR1 of SEQ ID NO:1, the CDR2 of SEQ ID NO: 4, and the CDR3 of SEQ ID NO: 11. This heavy chain, designated 10A1 VH S15G, comprises the amino acid of SEQ ID NO: 36 as follows:

(SEQ ID NO: 36, with CDRs in bold)
QVQLQQSGPVLVKPGQTLSLTCAISGDSISSDTTAWS
WIRRSPSRGLEWLGWTYYRSKWFNYYAVSVKGRVTIN
PDTSKNQFSLQLSSVTPEDTAVYYCARGNGNGGMDVW
GQGTTVTVSS

Another exemplary antibody-based molecule that binds CD99 includes a heavy chain that includes framework region 1 according to SEQ ID NO: 54 in combination with the CDR1 of SEQ ID NO:1, the CDR2 of SEQ ID NO: 8, and the CDR3 of SEQ ID NO: 14. This heavy chain, designated 10A1 VH S15G/T53I/N105A, comprises the amino acid of SEQ ID NO: 41 as follows:

(SEQ ID NO: 41, with CDRs in bold)
QVQLQQSGPVLVKPGQTLSLTCAISGDSISSDTTAWS
WIRRSPSRGLEWLGWIYYRSKWFNYYAVSVKGRVTIN
PDTSKNQFSLQLSSVTPEDTAVYYCARGNGAGGMDVW
GQGTTVTVSS

The above-identified heavy chain antibody fragments can be used in combination with any of the above-identified light chain antibody fragments.

In some embodiments, the heavy chain and/or the light chain variable regions of the antibody-based molecule described herein further comprises human or humanized immunoglobulin heavy chain and/or light chain framework regions, respectively.

In some embodiments, the CD99 antibody-based molecule comprises:
(i) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(ii) a heavy chain variable region comprising the CDR-H1 of SEQ ID NO: 1, the CDR-H2 of SEQ ID NO: 5, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 24;
(iii) a heavy chain variable region comprising the CDR-H1 of SEQ ID NO: 2, the CDR-H2 of SEQ ID NO: 6, and the CDR-H3 of SEQ ID NO: 12, and a light chain variable region comprising the CDR-L1 of SEQ ID NO: 16, the CDR-L2 of SEQ ID NO: 20, and the CDR-L3 of SEQ ID NO: 25;
(iv) a heavy chain variable region comprising the CDR-H1 of SEQ ID NO: 3, the CDR-H2 of SEQ ID NO: 7, and the CDR-H3 of SEQ ID NO: 13, and a light chain variable region comprising the CDR-L1 of SEQ ID NO: 17, the CDR-L2 of SEQ ID NO: 21, and the CDR-L3 of SEQ ID NO: 26;
(v) a heavy chain variable region comprising the CDR-H1 of SEQ ID NO: 1, the CDR-H2 of SEQ ID NO: 8, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(vi) a heavy chain variable region comprising the CDR-H1 of SEQ ID NO: 1, the CDR-H2 of SEQ ID NO: 9, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(vii) a heavy chain variable region comprising the CDR-H1 of SEQ ID NO: 1, the CDR-H2 of SEQ ID NO: 10, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(viii) a heavy chain variable region comprising the CDR-H1 of SEQ ID NO: 1, the CDR-H2 of SEQ ID NO: 4, and the CDR-H3 of SEQ ID NO: 14, and a light chain variable region comprising the CDR-L1 of SEQ ID NO: 15, the CDR-L2 of SEQ ID NO: 19, and the CDR-L3 of SEQ ID NO: 23;
(ix) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 18, the CDR-L2 of SEQ ID NO: 19, and the CDR-L3 of SEQ ID NO: 23;
(x) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 22, and the CDR-L3 of SEQ ID NO: 23;
(xi) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 27; and/or
(xii) a heavy chain variable region comprising the CDR-H1 of SEQ ID NO: 1, the CDR-H2 of SEQ ID NO: 8, and the CDR-H3 of SEQ ID NO: 14, and a light chain variable region comprising the CDR-L1 of SEQ ID NO: 15, the CDR-L2 of SEQ ID NO: 19, and the CDR-L3 of SEQ ID NO: 23.
   Further embodiments may include heavy chain or light chain variable regions containing one or more stability enhancing amino acid modifications including, without limitation, the heavy chain framework region according to SEQ ID NO: 54.

The CD99 antibody-based molecule as described herein may comprises a variable light (V_{L}) chain, a variable heavy (V_{H}) chain, or a combination of V_{L} and V_{H} chains. In some embodiments, the V_{H} chain of the CD99 antibody-based molecule comprises any one of the V_{H} amino acid sequences provided in Table 3 below, or an amino acid sequence that is at least 60%, at least 65%, at least 70%, at least 75%, at least 80%, at least 85%, at least 90%, at least 95% identical to any one of the V_{H} amino acid sequences listed in Table 3. For example, the antibody-based molecule described herein may comprise:
(i) a heavy chain variable region comprising an amino acid sequence that is at least 80% identical to SEQ ID NO: 28;
(ii) a heavy chain variable region comprising an amino acid sequence that is at least 80% identical to SEQ ID NO: 30;
(iii) a heavy chain variable region comprising an amino acid sequence that is at least 80% identical to SEQ ID NO: 32;
(iv) a heavy chain variable region comprising an amino acid sequence that is at least 80% identical to SEQ ID NO: 34;
(v) a heavy chain variable region comprising an amino acid sequence that is at least 80% identical to SEQ ID NO: 36;
(vi) a heavy chain variable region comprising an amino acid sequence that is at least 80% identical to SEQ ID NO: 37;
(viii) a heavy chain variable region comprising an amino acid sequence that is at least 80% identical to SEQ ID NO: 38;
(viii) a heavy chain variable region comprising an amino acid sequence that is at least 80% identical to SEQ ID NO: 39;
(ix) a heavy chain variable region comprising an amino acid sequence that is at least 80% identical to SEQ ID NO: 40; and/or
(x) a heavy chain variable region comprising an amino acid sequence that is at least 80% identical to SEQ ID NO: 41.

In some embodiments, the V_{L} chain of the CD99 antibody-based molecule comprises any one of the V_{L} amino acid sequences provided in Table 3 below, or an amino acid sequence that is at least 60%, at least 65%, at least 70%, at least 75%, at least 80%, at least 85%, at least 90%, at least 95% identical to any one of the V_{L} amino acid sequences listed in Table 3. For example, the antibody-based molecule described herein may comprise:
(i) a light chain variable region comprising an amino acid sequence that is at least 80% identical to SEQ ID NO: 29;
(ii) a light chain variable region comprising an amino acid sequence that is at least 80% identical to SEQ ID NO: 31;
(iii) a light chain variable region comprising an amino acid sequence that is at least 80% identical to SEQ ID NO: 33;
(iv) a light chain variable region comprising an amino acid sequence that is at least 80% identical to SEQ ID NO: 35;
(v) a light chain variable region comprising an amino acid sequence that is at least 80% identical to SEQ ID NO: 42;
(vi) a light chain variable region comprising an amino acid sequence that is at least 80% identical to SEQ ID NO: 43; and/or
(vii) a light chain variable region comprising an amino acid sequence that is at least 80% identical to SEQ ID NO: 44.

TABLE 3
CD99 Antibody Variable Heavy (VH) and Variable
Light (VL) Antibody Sequences

| mAb/ | | | |
|---|---|---|---|
| Fab | | | SEQ |
| clone | Re- | | ID |
| name | gion | Sequence* | NO: |
| 10A1 | V_{H} | QVQLQQSGPVLVKPSQTLSLTCAISGDSIS | 28 |
| | | SDTTAWSWIRRSPSRGLEWLGWTYYRSKWF | |
| | | NYYAVSVKGRVTINPDTSKNQFSLQLSSVT | |
| | | PEDTAVYYCARGNGNGGMDVWGQGTTVTVS | |
| | | S | |
| | V_{L} | SYVLTQPASASVSPGQTARITCSGDALPKQ | 29 |
| | | YAYWYQQKPGQAPVLVIFKDSERPSGVPER | |
| | | FSGSSSGTTVTLTISGAQAEDEADYYCQSV | |
| | | DNSGFYQVFGGGTQLTVL | |
| 10 | V_{H} | QVQLQQSGPVLVKPSQTLSLTCAISGDSVS | 30 |
| | | SDTTAWSWIRQSPSRGLEWLGWTYYRSKWF | |
| | | NYYAVSVKSRVTINPDTSKNQFSLQLSSVT | |
| | | PEDTAVYYCARGNGNGGMDVWGQGTTVTVS | |
| | | S | |
| | V_{L} | SYVLTQPPSVSVSPGQTARITCSGDALPKQ | 31 |
| | | YAYWYQQKPGQAPVLVIFKDSERPSGIPER | |
| | | FSGSSSGTTVTLTISGVQAEDEADYYCQSA | |
| | | DNSGFYQVFGGGTQLTVL | |
| 22 | V_{H} | QVQLQQSGPGLVKPSQTLSLTCAISGDSVS | 32 |
| | | SNSAAWNWIRQSPSRGLEWLGRTYYRSKWY | |
| | | NDYAVSVKSRITINPDTSKNQFSLQLNSVT | |
| | | PEDTAVYYCARGSYGSGLVWGQGTTVTVSS | |
| | V_{L} | NFMLTQPHSVSESPGKTVTISCTGSSGSIA | 33 |
| | | SNYVQWYQQRPGSAPTTVIYEDDQRPSGVP | |
| | | DRFSGSLDTSSNSASLTISGLRPEDEADYY | |
| | | CQSYDNKDNVVFGGGTKVTVL | |
| 30 | V_{H} | EVQLLETGGGLVQPGGSLRLSCAASGFTFS | 34 |
| | | SSAMHWVRQAPGKGLEWVAAISYDGSDTYY | |
| | | ADSVKGRFTISRDNAKNTLYLQMNSLRDED | |
| | | TAVYYCARDQVLGSGSLDYWGQGTLVTVSS | |
| | V_{L} | NFMLTQPHSVSESPGKTVTISCTRSSGSIG | 35 |
| | | SYYVQWYQQRPGSPPTTVIYEDDKRPSGVP | |
| | | DRFSGSIDSSSNSASLTISGLKTEDEADYY | |
| | | CQSYDSSHVVFGGGTKLTVL | |
| 10A1 | V_{H} | QVQLQQSGPVLVKPGQTLSLTCAISGDSIS | 36 |
| VH_ | | SDTTAWSWIRRSPSRGLEWLGWTYYRSKWF | |
| S15G | | NYYAVSVKGRVTINPDTSKNQFSLQLSSVT | |
| | | PEDTAVYYCARGNGNGGMDVWGQGTTVTVS | |
| | | S | |
| 10A1 | V_{H} | QVQLQQSGPVLVKPSQTLSLTCAISGDSIS | 37 |
| VH_ | | SDTTAWSWIRRSPSRGLEWLGWIYYRSKWF | |
| T53I | | NYYAVSVKGRVTINPDTSKNQFSLQLSSVT | |
| | | PEDTAVYYCARGNGNGGMDVWGQGTTVTVS | |
| | | S | |
| 10A1 | V_{H} | QVQLQQSGPVLVKPSQTLSLTCAISGDSIS | 38 |
| VH_ | | SDTTAWSWIRRSPSRGLEWLGWTYYRSAWF | |
| K58A | | NYYAVSVKGRVTINPDTSKNQFSLQLSSVT | |
| | | PEDTAVYYCARGNGNGGMDVWGQGTTVTVS | |
| | | S | |
| 10A1 | V_{H} | QVQLQQSGPVLVKPSQTLSLTCAISGDSIS | 39 |
| VH_ | | SDTTAWSWIRRSPSRGLEWLGWTYYRSKWA | |
| F60A | | NYYAVSVKGRVTINPDTSKNQFSLQLSSVT | |
| | | PEDTAVYYCARGNGNGGMDVWGQGTTVTVS | |
| | | S | |
| 10A1 | V_{H} | QVQLQQSGPVLVKPSQTLSLTCAISGDSIS | 40 |
| VH_ | | SDTTAWSWIRRSPSRGLEWLGWTYYRSKWF | |
| N105A | | NYYAVSVKGRVTINPDTSKNQFSLQLSSVT | |
| | | PEDTAVYYCARGNGAGGMDVWGQGTTVTVS | |
| | | S | |
| 10A1 | V_{H} | QVQLQQSGPVLVKPGQTLSLTCAISGDSIS | 41 |
| VH_ | | SDTTAWSWIRRSPSRGLEWLGWIYYRSKWF | |
| S15G/ | | NYYAVSVKGRVTINPDTSKNQFSLQLSSVT | |
| T53I/ | | PEDTAVYYCARGNGAGGMDVWGQGTTVTVS | |
| N105A | | S | |
| 10A1 | V_{L} | SYVLTQPASASVSPGQTARITCSGDALPAQ | 42 |
| VL_ | | YAYWYQQKPGQAPVLVIFKDSERPSGVPER | |
| K29A | | FSGSSSGTTVTLTISGAQAEDEADYYCQSV | |
| | | DNSGFYQVFGGGTQLTVL | |
| 10A1 | V_{L} | SYVLTQPASASVSPGQTARITCSGDALPKQ | 43 |
| VL_ | | YAYWYQQKPGQAPVLVIFADSERPSGVPER | |
| K49A | | FSGSSSGTTVTLTISGAQAEDEADYYCQSV | |
| | | DNSGFYQVFGGGTQLTVL | |
| 10A1 | V_{L} | SYVLTQPASASVSPGQTARITCSGDALPKQ | 44 |
| VL_ | | YAYWYQQKPGQAPVLVIFKDSERPSGVPER | |
| Y96A | | FSGSSSGTTVTLTISGAQAEDEADYYCQSV | |
| | | DNSGFAQVFGGGTQLTVL | |

*Complementarity-determining regions are shown in bold typeface.

In some embodiments, the CD99 antibody-based molecule disclosed herein comprises:
(i) a heavy chain variable region comprising an amino acid sequence that is at least 80% identical to SEQ ID NO: 28 and a light chain variable region comprising an amino acid sequence that is at least 80% identical to SEQ ID NO: 29;
(ii) a heavy chain variable region comprising an amino acid sequence that is at least 80% identical to SEQ ID NO: 30 and a light chain variable region comprising an amino acid sequence that is at least 80% identical to SEQ ID NO: 31;
(iii) a heavy chain variable region comprising an amino acid sequence that is at least 80% identical to SEQ ID NO: 32 and a light chain variable region comprising an amino acid sequence that is at least 80% identical to SEQ ID NO: 33;
(iv) a heavy chain variable region comprising an amino acid sequence that is at least 80% identical to SEQ ID NO: 34 and a light chain variable region comprising an amino acid sequence that is at least 80% identical to SEQ ID NO: 35;
(v) a heavy chain variable region comprising an amino acid sequence that is at least 80% identical to SEQ ID NO: 36 and a light chain variable region comprising an amino acid sequence that is at least 80% identical to SEQ ID NO: 29;
(vi) a heavy chain variable region comprising an amino acid sequence that is at least 80% identical to SEQ ID NO: 37 and a light chain variable region comprising an amino acid sequence that is at least 80% identical to SEQ ID NO: 29;
(vii) a heavy chain variable region comprising an amino acid sequence that is at least 80% identical to SEQ ID NO: 38 and a light chain variable region comprising an amino acid sequence that is at least 80% identical to SEQ ID NO: 29;
(viii) a heavy chain variable region comprising an amino acid sequence that is at least 80% identical to SEQ ID NO: 39 and a light chain variable region comprising an amino acid sequence that is at least 80% identical to SEQ ID NO: 29;
(ix) a heavy chain variable region comprising an amino acid sequence that is at least 80% identical to SEQ ID NO: 40 and a light chain variable region comprising an amino acid sequence that is at least 80% identical to SEQ ID NO: 29;
(x) a heavy chain variable region comprising an amino acid sequence that is at least 80% identical to SEQ ID NO: 41 and a light chain variable region comprising an amino acid sequence that is at least 80% identical to SEQ ID NO: 29;
(xi) a heavy chain variable region comprising an amino acid sequence that is at least 80% identical to SEQ ID NO: 28 and a light chain variable region comprising an amino acid sequence that is at least 80% identical to SEQ ID NO: 42;
(xii) a heavy chain variable region comprising an amino acid sequence that is at least 80% identical to SEQ ID NO: 28 and a light chain variable region comprising an amino acid sequence that is at least 80% identical to SEQ ID NO: 43; and/or
(xiii) a heavy chain variable region comprising an amino acid sequence that is at least 80% identical to SEQ ID NO: 28 and a light chain variable region comprising an amino acid sequence that is at least 80% identical to SEQ ID NO: 44.
   Further embodiments may include heavy chain or light chain variable regions containing one or more stability enhancing amino acid modifications including, without limitation, the heavy chain framework region according to SEQ ID NO: 54.

In some embodiments, the CD99 antibody-based molecule disclosed herein is a chimeric antibody or binding fragment thereof.

In some embodiments, the CD99 antibody-based molecule disclosed herein is a humanized antibody or binding fragment thereof.

In some embodiments, the CD99 antibody-based molecule disclosed herein is a monoclonal antibody or biding fragment thereof.

In some embodiments, the CD99 antibody-based molecule disclosed herein is a full-length antibody, an epitope-binding fragment of an antibody, or an antibody derivative. In some embodiments, the CD99 antibody-based molecule is an epitope binding fragment selected from a F(ab) fragment, a F(ab′) fragment, and F(ab′)2 fragment. In other embodiments, the CD99 antibody-based molecule is an antibody derivative selected from the group consisting of a scFv, a minibody, a diabody, a triabody, a tribody, and a tetrabody.

Within the context of the present invention, the CD99 antibody-based molecules may be multivalent CD99 antibody-based molecules. Thus, in some embodiments, the CD99 antibody-based molecules may be a bivalent, a trivalent, a tetravalent, a pentavalent, a hexavalent, a heptavalent, an octavalent, a nonavalent, a decavalent, or a dodecavalent antibody-based molecule. For example, in some embodiments, the CD99 antibody-based molecule is a tetravalent antibody-based molecule. In other embodiments, the CD99 antibody-based molecule is a dodecavalent antibody-based molecule.

In some embodiments, the CD99 antibody-based molecule is an IgG-based molecule.

In some embodiments, the CD99 antibody-based molecules of the present invention comprise engineered hIgG Fc domains. For example, the CD99 antibody-based molecules may comprise an hIgG1-P329G LALA or hIgG4-P329G SPLE domain (see, e.g., Schlothauer et al., “Novel Human IgG1 and IgG4 Fc-Engineered Antibodies with Completely Abolished Immune Effector Functions,” Protein Engineering, Design and Selection 29(10):457-466 (2016), which is hereby incorporated by reference in its entirety). Thus, in some embodiments, the antibody-based molecule is an IgG-based molecule.

In some embodiments, the CD99 antibody-based molecules of the present invention comprise a modified or mutated hIgG Fc domain. Exemplary IgG modifications or mutations include, without limitation, E345R and E345R/E430G/S440Y (see, e.g., Saunders, K., “Conceptual Approaches to Modulating Antibody Effector Functions and Circulation Half-Life,” Front. Immunol. (2019) and Diebolder et al., “Complement is Activated by IgG Hexamers Assembled at the Cell Surface,” Science 343(6176):1260-1263 (2014), which are hereby incorporated by reference in their entirety). The E345R and E345R/E430G/S440Y mutations result in hexameric IgG molecules linked via the Fc. Additional suitable IgG modifications or mutations include, without limitation, S298A/E33A/K334A, S239D/A330L/I332E, S239D/I332E, G236A/S239D/A330L/I332E, G236A, S239D/I332E/G236A, L234Y/G236W/S239A, F243L/R292P/Y300L/V305I/P396L, K326W/E333S, K326A/E333A, K326M/E333S, C221D/D222C, S267E/H268F/S324T, and H268F/S324T (see, e.g., Saunders, K., “Conceptual Approaches to Modulating Antibody Effector Functions and Circulation Half-Life,” Front. Immunol. (2019), which is hereby incorporated by reference in its entirety).

In some embodiments, the CD99 antibody-based molecules of the present invention comprise an Fc domain, said Fc domain comprising one or more amino acid residue substitutions that allow assembly of two or more antibody-based molecules into a molecular complex. For example, the antibody-based molecule may comprise an Fe domain comprising amino acid residues 122-451 of SEQ TD NO: 47 or amino acid residues 122-451 of SEQ TD NO: 48. The sequences of suitable CD99 Human IgG1 heavy chain complete sequences comprising one or more amino acid residue substitutions are provided in Table 4 below.

TABLE 4
CD99 Human IgG1 Heavy Chain Complete Sequences

| mAb/ | | |
|---|---|---|
| Fab | | SEQ |
| clone | | ID |
| name | Sequence* | NO: |
| 10A1 | QVQLQQSGPVLVKPSQTLSLTCAISGDSIS | 45 |
| LALA | SDTTAWSWIRRSPSRGLEWLGWTYYRSKWF | |
| | NYYAVSVKGRVTINPDTSKNQFSLQLSSVT | |
| | PEDTAVYYCARGNGNGGMDVWGQGTTVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPEAAG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALGAPIEKTISKAKGQPREPQVYTLPPSRE | |
| | EMTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| 10A1 | QVQLQQSGPVLVKPSQTLSLTCAISGDSIS | 46 |
| HC- | SDTTAWSWIRRSPSRGLEWLGWTYYRSKWF | |
| scFv | NYYAVSVKGRVTINPDTSKNQFSLQLSSVT | |
| | PEDTAVYYCARGNGNGGMDVWGQGTTVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPEAAG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALGAPIEKTISKAKGQPREPQVYTLPPSRE | |
| | EMTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | KSSGSSGSSSSSGSSSQVQLQQSGPVLVKP | |
| | SQTLSLTCAISGDSISSDTTAWSWIRRSPS | |
| | RGLEWLGWTYYRSKWFNYYAVSVKGRVTIN | |
| | PDTSKNQFSLQLSSVTPEDTAVYYCARGNG | |
| | NGGMDVWGQGTTVTVSSGSSSSSSGSSGSS | |
| | SSSYVLTQPASASVSPGQTARITCSGDALP | |
| | KQYAYWYQQKPGQAPVLVIFKDSERPSGVP | |
| | ERFSGSSSGTTVTLTISGAQAEDEADYYCQ | |
| | SVDNSGFYQVFGGGTQLTVL | |
| 10A1 | QVQLQQSGPVLVKPSQTLSLTCAISGDSIS | 47 |
| E345R | SDTTAWSWIRRSPSRGLEWLGWTYYRSKWF | |
| | NYYAVSVKGRVTINPDTSKNQFSLQLSSVT | |
| | PEDTAVYYCARGNGNGGMDVWGQGTTVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPEAAG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALGAPIEKTISKAKGQPRRPQVYTLPPSRE | |
| | EMTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| 10A1 | QVQLQQSGPVLVKPSQTLSLTCAISGDSIS | 48 |
| RGY | SDTTAWSWIRRSPSRGLEWLGWTYYRSKWF | |
| | NYYAVSVKGRVTINPDTSKNQFSLQLSSVT | |
| | PEDTAVYYCARGNGNGGMDVWGQGTTVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPEAAG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALGAPIEKTISKAKGQPRRPQVYTLPPSRE | |
| | EMTKNQVSLTCLVKGFYPSDIAVEWESNGQ | |
| | PENNYKTTPPVLDSDGSFFLYSKLTVDKSR | |
| | WQQGNVFSCSVMHGALHNHYTQKYLSLSPG | |
| | K | |
| | (CH2 and CH3 are shown in | |
| | italic.) | |

*Underlined amino acid residues are constant region mutations; complementarity-determining regions are shown in bold typeface.

In some embodiments, the CD99 antibody-based molecules of the present invention may further comprise one or more appended CD99-binding scFv portions. The one or more CD99-binding scFv portions may be appended to heavy chain(s) and/or light chain(s) of the IgG-based molecule. For example, the CD99 antibody-based molecule may be an IgG(H)-scFv or an scFv-(H)IgG.

In some embodiments, the antibody-based molecule is an IgG(H)-scFv, where said IgG(H)-scFv comprises a heavy chain amino acid sequence that is at least 80% identical the amino acid sequence of SEQ ID NO: 46 and a light chain amino acid sequence that is at least 80% identical to the amino acid sequence of SEQ ID NO: 49 (shown in Table 5 below).

TABLE 5
CD99 Human Kappa Light Chain Complete Sequences

| mAb/ | | |
|---|---|---|
| Fab | | SEQ |
| clone | | ID |
| name | Sequence* | NO: |
| 10A1 | SYVLTQPASASVSPGQTARITCSGDALPKQYAY | 49 |
| | WYQQKPGQAPVLVIFKDSERPSGVPERFSGSSS | |
| | GTTVTLTISGAQAEDEADYYCQSVDNSGFYQVF | |
| | GGGTQLTVLRTVAAPSVFIFPPSDEQLKSGTAS | |
| | VVCLLNNFYPREAKVQWKVDNALQSGNSQESVT | |
| | EQDSKDSTYSLSSTLTLSKADYEKHKVYACEVT | |
| | HQGLSSPVTKSFNRGEC | |

*Complementarity-determining regions are shown in bold typeface.

Additional CD99 antibody-based molecule sequences are shown in Table 6 below.

TABLE 6
CD99 scFv-Fc Complete Sequences

| mAb/ | | |
|---|---|---|
| Fab | | SEQ |
| clone | | ID |
| name | Sequence* | NO: |
| 10A1 | QVQLQQSGPVLVKPSQTLSLTCAISGDSIS | 50 |
| | SDTTAWSWIRRSPSRGLEWLGWTYYRSKWF | |
| | NYYAVSVKGRVTINPDTSKNQFSLQLSSVT | |
| | PEDTAVYYCARGNGNGGMDVWGQGTTVTVS | |
| | SGILSGSSSSGSSSSGSSSSGYVLTQPASA | |
| | SVSPGQTARITCSGDALPKQYAYWYQQKPG | |
| | QAPVLVIFKDSERPSGVPERFSGSSSGTTV | |
| | TLTISGAQAEDEADYYCQSVDNSGFYQVFG | |
| | GGTQLTVLSGSSSSLEGCKPCICTVPEVSS | |
| | VFIFPPKPKDVLTITLTPKVTCVVVDISKD | |
| | DPEVQFSWFVDDVEVHTAQTQPREEQFNST | |
| | FRSVSELPIMHQDWLNGKEFKCRVNSAAFP | |
| | APIEKTISKTKGRPKAPQVYTIPPPKEQMA | |
| | KDKVSLTCMITDFFPEDITVEWQWNGQPAE | |
| | NYKNTQPIMDTDGSYFVYSKLNVQKSNWEA | |
| | GNTFTCSVLHEGLHNHHTEKSLSHSPGKGG | |
| | GGLNDIFEAQKIEWHESRHHHHHH | |
| | *This construct contains the | |
| | S141G mutation, which is | |
| | converted back to S in the | |
| | IgG formats* | |
| 10 | QVQLQQSGPVLVKPSQTLSLTCAISGDSVS | 51 |
| | SDTTAWSWIRQSPSRGLEWLGWTYYRSKWF | |
| | NYYAVSVKSRVTINPDTSKNQFSLQLSSVT | |
| | PEDTAVYYCARGNGNGGMDVWGQGTTVTVS | |
| | SGILGSGGGGSGGGGSGGGGSSYVLTQPPS | |
| | VSVSPGQTARITCSGDALPKQYAYWYQQKP | |
| | GQAPVLVIFKDSERPSGIPERFSGSSSGTT | |
| | VTLTISGVQAEDEADYYCQSADNSGFYQVF | |
| | GGGTQLTVLSGSSSSLEGCKPCICTVPEVS | |
| | SVFIFPPKPKDVLTITLTPKVTCVVVDISK | |
| | DDPEVQFSWFVDDVEVHTAQTQPREEQFNS | |
| | TFRSVSELPIMHQDWLNGKEFKCRVNSAAF | |
| | PAPIEKTISKTKGRPKAPQVYTIPPPKEQM | |
| | AKDKVSLTCMITDFFPEDITVEWQWNGQPA | |
| | ENYKNTQPIMDTDGSYFVYSKLNVQKSNWE | |
| | AGNTFTCSVLHEGLHNHHTEKSLSHSPGKG | |
| | GGGLNDIFEAQKIEWHESRHHHHHH | |
| 22 | QVQLQQSGPGLVKPSQTLSLTCAISGDSVS | 52 |
| | SNSAAWNWIRQSPSRGLEWLGRTYYRSKWY | |
| | NDYAVSVKSRITINPDTSKNQFSLQLNSVT | |
| | PEDTAVYYCARGSYGSGLVWGQGTTVTVSS | |
| | GSASAPTGILGSGGGGSGGGGSGGGGSNFM | |
| | LTQPHSVSESPGKTVTISCTGSSGSIASNY | |
| | VQWYQQRPGSAPTTVIYEDDQRPSGVPDRF | |
| | SGSLDTSSNSASLTISGLRPEDEADYYCQS | |
| | YDNKDNVVFGGGTKVTVLSGSSSSLEGCKP | |
| | CICTVPEVSSVFIFPPKPKDVLTITLTPKV | |
| | TCVVVDISKDDPEVQFSWFVDDVEVHTAQT | |
| | QPREEQFNSTFRSVSELPIMHQDWLNGKEF | |
| | KCRVNSAAFPAPIEKTISKTKGRPKAPQVY | |
| | TIPPPKEQMAKDKVSLTCMITDFFPEDITV | |
| | EWQWNGQPAENYKNTQPIMDTDGSYFVYSK | |
| | LNVQKSNWEAGNTFTCSVLHEGLHNHHTEK | |
| | SLSHSPGKGGGGLNDIFEAQKIEWHESRHH | |
| | HHHH | |
| 30 | EVQLLETGGGLVQPGGSLRLSCAASGFTFS | 53 |
| | SSAMHWVRQAPGKGLEWVAAISYDGSDTYY | |
| | ADSVKGRFTISRDNAKNTLYLQMNSLRDED | |
| | TAVYYCARDQVLGSGSLDYWGQGTLVTVSS | |
| | GILGSGGGGSGGGGSGGGGSNFMLTQPHSV | |
| | SESPGKTVTISCTRSSGSIGSYYVQWYQQR | |
| | PGSPPTTVIYEDDKRPSGVPDRFSGSIDSS | |
| | SNSASLTISGLKTEDEADYYCQSYDSSHVV | |
| | FGGGTKLTVLSGSSSSLEGCKPCICTVPEV | |
| | SSVFIFPPKPKDVLTITLTPKVTCVVVDIS | |
| | KDDPEVQFSWFVDDVEVHTAQTQPREEQFN | |
| | STFRSVSELPIMHQDWLNGKEFKCRVNSAA | |
| | FPAPIEKTISKTKGRPKAPQVYTIPPPKEQ | |
| | MAKDKVSLTCMITDFFPEDITVEWQWNGQP | |
| | AENYKNTQPIMDTDGSYFVYSKLNVQKSNW | |
| | EAGNTFTCSVLHEGLHNHHTEKSLSHSPGK | |
| | GGGGLNDIFEAQKIEWHESRHHHHHH | |

*Complementarity-determining regions are shown in bold typeface.

The CD99 antibody-based molecules of the present invention may be described or specified in terms of their binding affinities. Thus, in some embodiments, the CD99 antibody-based molecules of the present invention include those with a dissociation constant or K_{D} less than 1 μM, 500 nM, 250 nM, 200 nM, 100 nM, 50 nM, 40 nM, 30 nM, 25 nM, 20 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM.

In some embodiments, the CD99 antibody-based molecules of the present invention are described or specified in terms of their cytotoxicity or IC₅₀ values. Thus, in some embodiments, the CD99 antibody-based molecules of the present invention include those with a cytotoxicity or IC₅₀ value against CD99-expressing cancer cells of less than 2 μM, 1 μM, 900 nM, 800 nM, 700 nM, 600 nM, 500 nM, 400 nM, 300 nM, 200 nM, 100 nM, 50 nM, 40 nM, 30 nM, 20 nM, 10 nM, 5 nM, 4 nM, 3 nM, 2 nM, or 1 nM.

Additional aspects of the present disclosure relate to isolated polynucleotides encoding the CD99 antibody-based molecules described herein. In one embodiment, the polynucleotide encoding the CD99 antibody of the present invention comprises a sequence encoding any one, any two, any three, any four, any five, or any six of the CDRs described supra, including the heavy chain CDRs of SEQ ID NOs: 1-14 and the light chain CDRs of SEQ ID NOs: 15-27.

In one embodiment, the polynucleotide comprises a nucleotide sequence encoding a V_{H} domain, where the V_{H} domain comprises:
(i) a heavy chain variable region comprising the CDR-H1 of SEQ ID NO: 1, the CDR-H2 of SEQ ID NO: 4, and the CDR-H3 of SEQ ID NO: 11;
(ii) a heavy chain variable region comprising the CDR-H1 of SEQ ID NO: 1, the CDR-H2 of SEQ ID NO: 5, and the CDR-H3 of SEQ ID NO: 11;
(iii) a heavy chain variable region comprising the CDR-H1 of SEQ ID NO: 2, the CDR-H2 of SEQ ID NO: 6, and the CDR-H3 of SEQ ID NO: 12;
(iv) a heavy chain variable region comprising the CDR-H1 of SEQ ID NO: 3, the CDR-H2 of SEQ ID NO: 7, and the CDR-H3 of SEQ ID NO: 13;
(v) a heavy chain variable region comprising the CDR-H1 of SEQ ID NO: 1, the CDR-H2 of SEQ ID NO: 8, and the CDR-H3 of SEQ ID NO: 11;
(vi) a heavy chain variable region comprising the CDR-H1 of SEQ ID NO: 1, the CDR-H2 of SEQ ID NO: 9, and the CDR-H3 of SEQ ID NO: 11;
(vii) a heavy chain variable region comprising the CDR-H1 of SEQ ID NO: 1, the CDR-H2 of SEQ ID NO: 10, and the CDR-H3 of SEQ ID NO: 11;
(viii) a heavy chain variable region comprising the CDR-H1 of SEQ ID NO: 1, the CDR-H2 of SEQ ID NO: 4, and the CDR-H3 of SEQ ID NO: 14; and/or
(ix) a heavy chain variable region comprising the CDR-H1 of SEQ ID NO: 1, the CDR-H2 of SEQ ID NO: 8, and the CDR-H3 of SEQ ID NO: 14.

The sequences of the heavy chain CDR sequences are provided in Table 1 supra. Further embodiments may include heavy chain variable regions containing one or more stability enhancing amino acid modifications including, without limitation, the heavy chain framework region according to SEQ ID NO: 54.

In one embodiment, the polynucleotide comprises a nucleotide sequence encoding a V_{L} domain, where the V_{L} domain comprises:
(i) a light chain variable region comprising the CDR-L1 of SEQ ID NO: 15, the CDR-L2 of SEQ ID NO: 19, and the CDR-L3 of SEQ ID NO: 23;
(ii) a light chain variable region comprising the CDR-L1 of SEQ ID NO: 15, the CDR-L2 of SEQ ID NO: 19, and the CDR-L3 of SEQ ID NO: 24;
(iii) a light chain variable region comprising the CDR-L1 of SEQ ID NO: 16, the CDR-L2 of SEQ ID NO: 20, and the CDR-L3 of SEQ ID NO: 25;
(iv) a light chain variable region comprising the CDR-L1 of SEQ ID NO: 17, the CDR-L2 of SEQ ID NO: 21, and the CDR-L3 of SEQ ID NO: 26;
(v) a light chain variable region comprising the CDR-L1 of SEQ ID NO: 18, the CDR-L2 of SEQ ID NO: 19, and the CDR-L3 of SEQ ID NO: 23;
(vi) a light chain variable region comprising the CDR-L1 of SEQ ID NO: 15, the CDR-L2 of SEQ ID NO: 22, and the CDR-L3 of SEQ ID NO: 23; and/or
(vii) a light chain variable region comprising the CDR-L1 of SEQ ID NO: 15, the CDR-L2 of SEQ ID NO: 19, and the CDR-L3 of SEQ ID NO: 27.
   The sequences of the light chain CDR sequences are provided in Table 2 supra.

In one embodiment, the isolated polynucleotide encoding the CD99 antibody based molecule encodes any one of the V_{H} and/or V_{L} domain sequences as provided in Table 3 supra, or an amino acid sequence that is at least 60%, at least 65%, at least 70%, at least 75%, at least 80%, at least 85%, at least 90%, at least 95% identical to any one of the V_{H} and/or V_{L} amino acid sequences listed in Table 3. For example, the isolated polynucleotide encoding the CD99 antibody based molecule described herein may encode:
(i) a heavy chain variable region comprising an amino acid sequence that is at least 80% identical to SEQ ID NO: 28;
(ii) a heavy chain variable region comprising an amino acid sequence that is at least 80% identical to SEQ ID NO: 30;
(iii) a heavy chain variable region comprising an amino acid sequence that is at least 80% identical to SEQ ID NO: 32;
(iv) a heavy chain variable region comprising an amino acid sequence that is at least 80% identical to SEQ ID NO: 34;
(v) a heavy chain variable region comprising an amino acid sequence that is at least 80% identical to SEQ ID NO: 36;
(vi) a heavy chain variable region comprising an amino acid sequence that is at least 80% identical to SEQ ID NO: 37;
(viii) a heavy chain variable region comprising an amino acid sequence that is at least 80% identical to SEQ ID NO: 38;
(viii) a heavy chain variable region comprising an amino acid sequence that is at least 80% identical to SEQ ID NO: 39;
(ix) a heavy chain variable region comprising an amino acid sequence that is at least 80% identical to SEQ ID NO: 40; and/or
(x) a heavy chain variable region comprising an amino acid sequence that is at least 80% identical to SEQ ID NO: 41.
   Further embodiments may include heavy chain variable regions containing one or more stability enhancing amino acid modifications including, without limitation, the heavy chain framework region according to SEQ ID NO: 54.

In some embodiments, the isolated polynucleotide encoding the CD99 antibody based molecule described herein may encode:
(i) a light chain variable region comprising an amino acid sequence that is at least 80% identical to SEQ ID NO: 29;
(ii) a light chain variable region comprising an amino acid sequence that is at least 80% identical to SEQ ID NO: 31;
(iii) a light chain variable region comprising an amino acid sequence that is at least 80% identical to SEQ ID NO: 33;
(iv) a light chain variable region comprising an amino acid sequence that is at least 80% identical to SEQ ID NO: 35;
(v) a light chain variable region comprising an amino acid sequence that is at least 80% identical to SEQ ID NO: 42;
(vi) a light chain variable region comprising an amino acid sequence that is at least 80% identical to SEQ ID NO: 43; and/or
(vii) a light chain variable region comprising an amino acid sequence that is at least 80% identical to SEQ ID NO: 44.

The nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

The polynucleotides of the invention may be produced by chemical synthesis such as solid phase polynucleotide synthesis on an automated polynucleotide synthesizer and assembled into complete single or double stranded molecules. Alternatively, the polynucleotides of the invention may be produced by other techniques such as PCR followed by routine cloning. Techniques for producing or obtaining polynucleotides of a given sequence are well known in the art.

The polynucleotides of the invention may comprise at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as pIX or pIII. As is well appreciated by persons of skill in the art, recombinant expression of the polynucleotides can be enhanced by codon optimization for a particular host cell/organism.

Other aspects of the disclosure relate to vectors comprising at least one polynucleotide encoding the CD99 antibody-based molecule as described herein. Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. In one embodiment, the polynucleotide sequence encoding the heavy chain variable domain, alone or together with the polynucleotide sequence encoding the light chain variable domain as described herein, are combined with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the CD99 antibodies described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector (Vitale et al., “Anti-tau Conformational scFv MC1 Antibody Efficiently Reduces Pathological Tau Species in Adult JNPL3 Mice,” Acta. Neuropathol. Commun. 6(1):82 (2018), which is hereby incorporate by reference in its entirety), optionally containing the GFAP or CAG promoter and the Woodchuck hepatitis virus (WPRE) post-translational regulatory element. In another embodiment, the AAV vector is an AAV9 vector (Haiyan et al., “Targeting Root Cause by Systemic scAAV9-hIDS Gene Delivery: Functional Correction and Reversal of Severe MPSII in 20 Mice,” Mol. Ther. Methods Clin. Dev. 10:327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety).

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or -2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J Exp. Med. 214(5):1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety).

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89: 3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8: 609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34: 204-209 (2016) and Chan et al., Nat. Neurosci. 20: 1172-1179 (2017), which are hereby incorporated by reference in their entirety.

In accordance with this embodiment, the expression vector construct encoding the CD99 antibody-based molecule can include the polynucleotide sequence encoding the heavy chain polypeptide, a fragment thereof, a variant thereof, or combinations thereof. The expression construct can also include a nucleic acid sequence encoding the light chain polypeptide, a fragment thereof, a variant thereof, or combinations thereof.

The expression construct also typically comprises a promoter sequence suitable for driving expression of the CD99 antibody-based molecule. Suitable promoter sequences include, without limitation, the elongation factor 1-alpha promoter (EFla) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

The expression construct can further encode a linker sequence. The linker sequence can encode an amino acid sequence that spatially separates and/or links the one or more components of the expression construct (heavy chain and light chain components of the encoded antibody).

Further aspects of the disclosure relate to a host cell comprising a vector described herein. The CD99 antibody-based molecule described herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY (1987-2001); Sambrook et al., Molecular Cloning: A Laboratory Manual, 2^{nd} Edition, Cold Spring Harbor, N Y (1989); Harlow and Lane, Antibodies, a Laboratory Manual, Cold Spring Harbor, N Y (1989); Colligan et al., eds., Current Protocols in Immunology, John Wiley & Sons, Inc., NY (1994-2001); Colligan et al., Current Protocols in Protein Science, John Wiley & Sons, NY (1997-2001), which are hereby incorporated by reference in their entirety).

In some embodiments, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP2/0 cells, HeLa cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21(DE3), BL21-GOLD(DE3), XL1-Blue, JM109, HMS174, HMS174(DE3), and any of the natural or engineered Escherichia spp, Klebsiella spp., or Pseudomonas spp strains.

The CD99 antibody-based molecules described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells is sometimes preferable, and sometimes preferable in mammalian host cells, because such eukaryotic cells (and in particular mammalian cells) are more likely than prokaryotic cells to assemble and secrete a properly folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77: 4216-4220 (1980), which is hereby incorporated by reference in its entirety). Other suitable mammalian host cells include, without limitation, NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present disclosure. For example, it may be desirable to transfect a host cell with DNA encoding functional fragments of either the light chain and/or the heavy chain of an antibody described herein. Recombinant DNA technology may also be used to remove some or all of the DNA encoding either or both of the light and heavy chains that is not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

### Pharmaceutical Compositions Comprising CD99 Antibody-Based Molecules

Another aspect of the disclosure relates to a pharmaceutical composition comprising an antibody-based molecule, a polynucleotide, or a vector of the present disclosure and a pharmaceutically acceptable carrier.

The CD99 antibody-based molecules, the polynucleotide encoding the CD99 antibody-based molecules, and/or the vector comprising the isolated polynucleotide of the present disclosure are advantageously administered as pharmaceutical compositions comprising an active therapeutic agent (i.e., the CD99 antibody) and one or more of a variety of other pharmaceutically acceptable components. See REMINGTON: THE SCIENCE AND PRACTICE OF PHARMACY (21^{st} Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present invention (e.g., less than a substantial impact (e.g., 10% or less relative inhibition, 5% or less relative inhibition, etc.) on antigen binding).

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The compounds of the present invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the compounds of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient. An exemplary composition comprises an scFv at about 5 mg/mL, formulated in aqueous buffer consisting of 50 mM L-histidine, 150 mM NaCl, adjusted to pH 6.0 with HCl.

Typically, compositions are thus prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer et al., Science 249:1527 (1990); Hanes et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

### Administration of the Pharmaceutical Compositions Comprising CD99 Antibody Based Molecules

The CD99 antibody-based molecules of the present disclosure can be administered by parenteral, topical, oral or intranasal means for therapeutic treatment. Intramuscular injection (for example, into the arm or leg muscles) and intravenous infusion are preferred methods of administration of the molecules of the present invention. In some methods, such molecules are administered as a sustained release composition or device, such as a Medipad™ device (Elan Pharm. Technologies, Dublin, Ireland). In some methods, the molecules of the present invention are injected directly into a particular tissue where deposits have accumulated, for example intracranial injection.

In one embodiment, a pharmaceutical composition of the present invention is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

In therapeutic applications (i.e., in applications involving a patient who has been diagnosed with a CD99-expressing cancer such as T-cell acute lymphoblastic leukemia (T-ALL), acute myeloid leukemia (AML), acute lymphocytic leukemia, chronic myeloid leukemia (CML), and chronic lymphocytic leukemia (CLL)) the CD99 antibody based molecules of the present invention are administered to such patient in an amount sufficient to cure, treat, or at least partially arrest, the symptoms of the disease (as adduced by biochemical, histologic and/or behavioral assessment), including its complications and intermediate pathological phenotypes in development of the disease. In some embodiments, the administration of the therapeutic molecules of the present invention reduces or eliminates the number of CD99-expressing cancer cells in the subject relative to the number of CD99-expressing cancer cells in the subject prior to said administering.

Effective doses of the provided therapeutic molecules of the present invention, for the treatment of the above-described conditions may vary depending upon many different factors, including means of administration, target site, physiological state of the patient, other medications administered. Treatment dosages are typically titrated to optimize their safety and efficacy. On any given day that a dosage is given, the dosage of the CD99 antibody-based molecules as described herein may range from about 0.0001 to about 100 mg/kg, and more usually from about 0.01 to about 5 mg/kg, of the patient's body weight. For example, dosages can be 1 mg/kg body weight or 10 mg/kg body weight or within the range of 1-10 mg/kg body weight. Exemplary dosages thus include: from about 0.1 to about 10 mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight.

A physician or veterinarian having ordinary skill in the art may readily determine and prescribe the effective amount of the pharmaceutical composition required. For example, the physician or veterinarian could start doses of antibody-based molecule in the pharmaceutical composition at levels lower than that required in order to achieve the desired therapeutic effect and gradually increase the dosage until the desired effect is achieved. In general, a suitable daily dose of a composition of the present invention will be that amount of the compound which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the antibody-based molecule of the present invention to be administered alone, it is preferable to administer the antibody-based molecule as a pharmaceutical composition as described above.

For therapeutic purposes, the CD99 antibody-based molecules of the present disclosure are usually administered on multiple occasions. Intervals between single dosages (e.g., a bolus or infusion) can be weekly, monthly, or yearly. In some methods, dosage is adjusted to achieve a plasma concentration of 1-1000 μg/mL and in some methods 25-300 μg/mL. Alternatively, the therapeutic molecules of the present invention can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and non-human antibodies. scFv molecules generally have short serum half-lives.

In another embodiment, a pharmaceutical composition comprising a recombinant nucleic acid sequence encoding the CD99 antibody-based molecule as described herein, is administered to a subject to facilitate in vivo expression and formation of the antibody-based molecule for the treatment of conditions mediated by reduced signaling and/or phosphorylation of CD99. Expression vector constructs suitable for use in this embodiment of the disclosure are described supra.

The polynucleotide compositions can result in the generation of the CD99 antibody-based molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the antibody-based molecule in the subject within at least about 1 day, 2 days, 3 days, 4 days, 5 days, 6 days, 7 days, 8 days, 9 days, or 10 days of administration of the composition to the subject. The composition can result in generation of the antibody-based molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the persistent generation of the antibody-based molecule in the subject. The composition can result in the generation of the antibody-based molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

### Therapeutic Utility of the CD99-Binding Antibody-Based Molecules

Another aspect of the disclosure relates to a method of inducing apoptosis in a population of CD99-expressing cancer cells. This method involves contacting a population of CD99-expressing cancer cells with a therapeutically effective amount of a CD99 antibody-based molecule as described herein, an isolated polynucleotide encoding the antibody-based molecule as described herein, a vector comprising the polynucleotide as described herein, or a pharmaceutical composition as described herein, in an amount effective to induce apoptosis in the population of CD99-expressing cancer cells.

In some embodiments, the CD99 expressing cancer cells are from a haematopoietic or lymphoid malignancy.

The population of CD99-expressing cancer cells may be selected from a population of Ewing's sarcoma cells, T-cell acute lymphoblastic leukemia (T-ALL) cells, osteosarcoma cells, chondrosarcoma cells, glioma cells, melanoma cells, mesothelioma cells, leukemia cells, prostate cancer cells, liver cancer cells, Hodgkin's lymphoma cells, soft tissue cancer cells, thyroid cancer cells, kidney cancer cells, urinary tract cancer cells, colorectal cancer cells, upper aerodigestive tract cancer cells, B-cell acute lymphoblastic leukemia (B-ALL) cells, non-small cell lung cancer cells, stomach cancer cells, acute myeloid leukemia (AML) cells, endometrial cancer cells, lymphoma cells, breast cancer cells, meningioma cells, chronic myeloid leukemia (CML) cells, ovarian cancer cells, bile duct cancer cells, neuroblastoma cells, pancreatic cancer cells, medulloblastoma cells, esophageal cancer cells, Burkitt's lymphoma cells, small cell lung cancer cells, diffuse large B-cell lymphoma cells, chronic lymphocytic leukemia (CLL) cells, myelodysplastic syndrome (MDS) blast cells, and multiple myeloma cells.

In some embodiments, the antibody-based molecule is a bivalent, trivalent, tetravalent, or dodecavalent antibody-based molecule.

In accordance with this method, the composition is administered in an amount effective to induce apoptosis in CD99-expressing cancer cells in a subject. Such administration may be provided to a subject having a CD99-expressing cancer, such as a hematological cancer.

Thus, another aspect of the present disclosure relates to a method of treating a CD99-expressing cancer in a subject. This method involves administering, to a subject having a CD99-expressing cancer, an antibody-based molecule as described herein, an isolated polynucleotide encoding the antibody-based molecule as described herein, a vector comprising the polynucleotide as described herein, or a pharmaceutical composition as described herein, in an amount effective to treat the cancer in the subject.

In some embodiments of the methods described herein, the CD99-expressing cancer is a haematopoietic or lymphoid malignancy.

The CD99-expressing cancer may be selected from Ewings sarcoma, T-cell acute lymphoblastic leukemia (T-ALL), osteosarcoma, chondrosarcoma, glioma, melanoma, mesothelioma, leukemia, prostate cancer, liver cancer, Hodgkin's lymphoma, soft tissue cancer, thyroid cancer, kidney cancer, urinary tract cancer, colorectal cancer, upper aerodigestive tract cancer, B-cell acute lymphoblastic leukemia (B-ALL), non-small cell lung cancer, stomach cancer, acute myeloid leukemia (AML), endometrial cancer, lymphoma, breast cancer, meningioma, chronic myeloid leukemia (CML), ovarian cancer, bile duct cancer, neuroblastoma, pancreatic cancer, medulloblastoma, esophageal cancer, Burkitt's lymphoma, small cell lung cancer, diffuse large B-cell lymphoma, chronic lymphocytic leukemia (CLL), myelodysplastic syndrome (MDS), and multiple myeloma.

In some embodiments, the antibody-based molecule is a bivalent, trivalent, tetravalent, or dodecavalent antibody-based molecule.

There are several types of CD99-expressing cancers which may be treated in accordance with the method and compositions a described herein. In some embodiments, the cancer is a “carcinoma”, which is a cancer that begins in the skin or in tissues that line or cover internal organs. In other embodiments, the cancer is a “sarcoma”, which is a cancer that begins in bone, cartilage, fat, muscle, blood vessels, or other connective or supportive tissue. In other embodiments, the cancer is a “leukemia” which is a cancer that starts in blood-forming tissue, such as the bone marrow, and causes large numbers of abnormal blood cells to be produced and enter the blood. In other embodiments, the cancer is a “lymphoma” or “multiple myeloma”, which are cancers that begin in the cells of the immune system. In other embodiments, the cancer is a central nervous system cancer, which is a cancer that begins in the tissues of the brain and spinal cord.

In some embodiments, the hematological cancer is a leukemia. In other embodiments, the hematological cancer is a myeloma. In further embodiments, the hematological cancer is a lymphoma.

In some embodiments, the CD99-expressing cancer is selected from the group consisting of acute myeloid leukemia (AML), acute lymphocytic leukemia, chronic myeloid leukemia (CML), and chronic lymphocytic leukemia (CLL). In other embodiments, the CD99-expressing cancer is myelodysplastic syndrome (MDS).

Acute myeloid leukemia (AML) and the myelodysplastic syndromes (MDS) are disorders arising in the hematopoietic system that share common molecular origins, with MDS patients showing a propensity to progress to AML (Chung et al., “CD99 is a Therapeutic Target on Disease Stem Cells in Myeloid Malignancies,” Sci. Transl. Med. 9(374): eaaj2025 (2017), which is hereby incorporated by reference in its entirety). AML arises from immature hematopoietic cells and is composed of leukemic blasts organized in a developmental hierarchy reminiscent of normal hematopoiesis. At the apex of this hierarchy are leukemic stem cells (LSCs) that possess the capacity to self-renew and differentiate into non-self renewing progeny that comprise the vast majority of leukemic blasts (Bonnet and Dick, “Human Acute Myeloid Leukemia is Organized as a Hierarchy that Originates from a Primitive Hematopoietic Cell,” Nature Medicine 3:730-737 (1997) and Lapidot et al., “A Cell Initiating Human Acute Myeloid Leukaemia After Transplantation into SCID Mice,” Nature 367:645-648 (1994), which are hereby incorporated by reference in their entirety). LSCs appear to be largely resistant to conventional chemotherapy and are thought to serve as the reservoir of minimal residual disease (MRD) that is responsible for disease relapse following initial treatment (Ishikawa et al., “Chemotherapy-Resistant Human AML Stem Cells Home to and Engraft with the Bone-Marrow Endosteal Region,” Nature Biotechnology 25:1315-1321 (2007), which is hereby incorporated by reference in its entirety). Xenografts and mouse models of AML suggest that fully transformed LSCs arise not from hematopoietic stem cells (HSCs), but rather more committed myeloid progenitors that acquire aberrant self-renewal (Krivtsov et al., “Transformation from Committed Progenitor to Leukaemia Stem Cell Initiated by MLL-AF9,” Nature 442:818-822 (2006); Goardon et al., “Coexistence of LMPP-Like and GMP-Like Leukemia Stem Cells in Acute Myeloid Leukemia.” Cancer Cell 19:138-152 (2011); and Jamieson et al., “Granulocyte-Macrophage Progenitors as Candidate Leukemic Stem Cells in Blast-Crisis CML,” The New England Journal of Medicine 351:657-667 (2004), which are hereby incorporated by reference in their entirety). In contrast, MDS is initiated by neoplastic HSCs that fail to give rise to sufficient numbers of mature hematopoietic cells, leading to bone marrow failure (Pang et al., “Hematopoietic Stem Cell and Progenitor Cell Mechanisms in Myelodysplastic Syndromes,” Proc. Natl. Acad. Sci. USA 110:3011-3016 (2013); Will et al., “Stem and Progenitor Cells in Myelodysplastic Syndromes show Aberrant Stage-Specific Expansion and Harbor Genetic and Epigenetic Alterations,” Blood 120:2076-2086 (2012); and Woll et al., “Myelodysplastic Syndromes are Propagated by Rare and Distinct Human Cancer Stem Cells in vivo,” Cancer Cell 25:794-808 (2014), which are hereby incorporated by reference in their entirety). True to their identity as disease stem cells, when transplanted into immunodeficient animals MDS HSCs give rise to long-term grafts that recapitulate features of MDS (Pang et al., “Hematopoietic Stem Cell and Progenitor Cell Mechanisms in Myelodysplastic Syndromes,” Proc. Natl. Acad. Sci. USA 110:3011-3016 (2013) and Medyouf et al., “Myelodysplastic Cells in Patients Reprogram Mesenchymal Stromal Cells to Establish a Transplantable Stem Cell Niche Disease Unit,” Cell Stem Cell. 14(6):824-837 (2014), which are hereby incorporated by reference in their entirety). Similar to AML LSCs, MDS HSCs are also highly resistant to standard therapies (Will et al., “Stem and Progenitor Cells in Myelodysplastic Syndromes Show Aberrant Stage-Specific Expansion and Harbor Genetic and Epigenetic Alterations,” Blood 120:2076-2086 (2012) and Tehranchi et al., “Persistent Malignant Stem Cells in del(5q) Myelodysplasia in Remission,” The New England Journal of Medicine 363:1025-1037 (2010), which are hereby incorporated by reference in their entirety). Thus, despite the likely differing cellular origins of AML and MDS, curative therapies for these malignancies must eliminate disease stem cells (LSCs or MDS HSCs, respectively), as they are likely the only self-renewing disease cells in the bone marrow (BM).

In some embodiments, the CD99-expressing cancer is selected from the group consisting of breast cancer, colon cancer, prostate cancer, synovial sarcoma, Ewing's sarcoma, and osteosarcoma.

The CD99-expressing cancer may be a breast cancer. As used herein, a “breast cancer” refers to a condition characterized by anomalous rapid proliferation of abnormal cells that originate in the breast of a subject. Breast cancer cells may be identified in one or both breasts only and not in another tissue or organ, in one or both breasts and one or more adjacent tissues or organs (e.g., lymph node), or in one or both breasts and one or more non-adjacent tissues or organs to which the breast cancer cells have metastasized.

In some embodiments, the breast cancer is characterized by its progesterone receptor and human epidermal growth factor receptor 2 (HER2) status. For example, the breast cancer may be a progesterone receptor positive (PR⁺) or progesterone receptor negative (PR⁻) malignancy. In another embodiment, the breast cancer is human epidermal growth factor receptor 2 positive (HER2⁺) or human epidermal growth factor receptor 2 negative (HER2⁻) cancer. In another embodiment, the breast cancer is an androgen receptor positive (AR⁺) or androgen receptor negative (AR⁻) cancer.

In some embodiments, the breast cancer is classified as an estrogen receptor positive (ER⁺) or an estrogen receptor negative (ER⁻) cancer. ER⁺ breast cancers are cancers where active ER signaling drives proliferation. There are two major isoforms of estrogen receptor, ERα and ERβ. ERα and ERβ are encoded by two unique genes that reside on distinct chromosomes and each isoform is responsible for the regulation of a specific set of genes that elicit tissue-specific effects. The role of ERα in cancer initiation and progression has been well established in breast cancer (Fullwood et al., “An Oestrogen-Receptor-a-Bound Human Chromatin Interactome,” Nature 462:58-64 (2009); Sommer et al., “Estrogen Receptor and Breast Cancer,” Semin. Cancer Biol. 11:339-352 (2001); Sommer et al., “Estrogen Receptor and Breast Cancer,” Semin. Cancer Biol. 11:339-52 (2001); Oxelmark et al., “The Cochaperone p23 Differentially Regulates Estrogen Receptor Target Genes and Promotes Tumor Cell Adhesion and Invasion,” Mol. Cell. Biol. 26:5205-13 (2006); Simpson et al., “High Levels of Hsp90 Cochaperone p23 Promote Tumor Progression and Poor Prognosis in Breast Cancer by Increasing Lymph Node Metastases and Drug Resistance,” Cancer Res. 70:8446-56 (2010), each of which are hereby incorporated by reference in their entirety).

In some embodiments of the methods described herein, the breast cancer is an HER2⁺/ER⁻ breast cancer. The presence and/or absence of HER2 and ER (as well as other hormone receptors) in breast cancers or tumor cells can be readily evaluated, e.g., by immunohistochemistry (IHC). Certain embodiments of the methods disclosed herein further comprise determining that the tumor expresses HER2 and optionally one or more other receptors (e.g., PR, HER2, AR).

In some embodiments of the methods described herein, the CD99-expressing cancer is a synovial sarcoma. Synovial sarcoma is a cancer that can come from different types of soft tissue, such as muscle or ligaments. It is often found in the arm, leg, or foot, and near joints such as the wrist or ankle.

In some embodiments of the methods disclosed herein, the CD99-expressing cancer is a bone cancer. Bone cancers that can be treated in accordance with the methods herein include, but are not limited to, osteosarcoma, chondrosarcoma (dedifferentiated chondrosarcoma, clear cell chondrosarcoma, mesenchymal chondrosarcoma), Ewing sarcoma, malignant fibrous histiocytoma, fibrosarcoma, giant cell tumor of bone, chordoma, non-Hodgkin lymphoma, and multiple myeloma. Ewing's sarcoma is an aggressive cancer that occurs in bones or in the soft tissue around bones.

In some embodiments of the methods disclosed herein, the CD99-expressing cancer is a colorectal cancer. Colorectal cancers that can be treated in accordance with the methods herein include cancers that originate in the colon and rectum. Exemplary colon cancers include, but are not limited to, adenocarcinoma, carcinoid tumors, gastrointestinal stromal tumors, lymphomas, and sarcomas. Exemplary rectal cancers include, but are not limited to, adenocarcinoma, carcinoid tumors, gastrointestinal stromal tumors, lymphomas, and sarcomas.

In some embodiments of the methods disclosed herein, the CD99-expressing cancer is a prostate cancer. Prostate cancers that can be treated in accordance with the methods herein include, but are not limited to, adeonocarcinoma, sarcoma, small cell carcinomas, neuroendocrine tumors (other than small cell carcinomas), and transitional cell carcinomas.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring CD99, etc.

In accordance with this aspect of the disclosure, such administration treats the CD99-expressing cancer. The term “treatment” or “treating” as used herein means ameliorating, slowing or reversing the progress or severity of a disease or disorder, or ameliorating, slowing or reversing one or more symptoms or side effects of such disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of the progression a disease or disorder state, amelioration or palliation of a disease or disorder state, and remission of a disease or disorder, whether partial or total, detectable or undetectable.

An “effective amount,” of the antibody-based molecule refers to an amount sufficient, at dosages and for periods of time necessary, to achieve an intended biological effect or a desired therapeutic result including, without limitation, clinical results. The phrase “therapeutically effective amount” when applied to an antibody-based molecule of the invention is intended to denote an amount of the antibody that is sufficient to ameliorate, palliate, stabilize, reverse, slow or delay the progression of a disorder or disease state, or of a symptom of the disorder or disease. In an embodiment, the method of the present invention provides for administration of the antibody-based molecule in combinations with other compounds. In such instances, the “effective amount” is the amount of the combination sufficient to cause the intended biological effect.

Exemplary CD99 antibody-based molecules of the disclosure are shown in Tables 4-6, supra.

### EXAMPLES

The examples below are intended to exemplify the practice of embodiments of the disclosure but are by no means intended to limit the scope thereof.

### Materials and Methods for Examples 1-6

Target Cloning, Expression, and Purification from Escherichia coli:

GFP, SUMO, and GFP and SUMO fused with the extracellular domain of CD99 (ECD; residues 23-122 of UniProt ID P14209) containing a 6×His tag, AviTag, and TEV cleavage site at the N-Terminal region were cloned into pHBT plasmids (Sha et al., “Dissection of the BCR-ABL Signaling Network using Highly Specific Monobody Inhibitors to the SHP2 SH2 Domains,” Proc. Natl. Acad. Sci. USA 110:14924-14929 (2013), which is hereby incorporated by reference in its entirety). BL21(DE3) cells were transformed, separately, with plasmids. The target proteins were expressed via isopropyl β-D-1-thiogalactopyranoside (IPTG) induction at 18° C. for 20 hours. The targets were purified via immobilized metal affinity chromatography using a Ni-Sepharose resin (Cytivia). The purified proteins were biotinylated in vitro using in-house prepared recombinant BirA enzyme and further purified by size-exclusion chromatography using a Superdex 75 10/300 GL column (Cytivia).

### Single Chain Fv Selection Using Yeast Display:

Purified biotinylated targets were used for yeast display selection from a naïve scFv display library (Feldhaus et al., “Flow-Cytometric Isolation of Human Antibodies from a Nonimmune Saccharomyces cerevisiae Surface Display Library,” Nat. Biotechnol. 21:163-170 (2003), which is hereby incorporated by reference in its entirety) as previously described (Chao et al., “Isolating and Engineering Human Antibodies using Yeast Surface Display,” Nat. Protoc. 1:755-768 (2006) and Ackerman et al., “Highly Avid Magnetic Bead Capture: An Efficient Selection Method for de novo Protein Engineering Utilizing Yeast Surface Display,” Biotechnol. Prog. 25: 774-783 (2009), which are hereby incorporated by reference in their entirety). Briefly, in the first round of selection Dynabeads M-280 Streptavidin (ca #11206D, Invitrogen) were coated with biotinylated SUMO-CD99 and selection was performed by magnetic bead capture of target binding yeast clones. Yeast clones were then expanded, and two rounds of fluorescent activated cell sorting (FACS) was performed. In the first round, 100 nM NeutrAvidin 650 (ca #84607, Invitrogen) complexed with SUMO-CD99 tetramer was incubated with the library then sorted. In the second round, 20 nM of Streptavidin 650 (ca #84547, Invitrogen) complexed with GFP-CD99 tetramer was incubated with the library then sorted. Cell sorting was performed on a S3e Cell Sorter (Bio-Rad).

Detection of scFv expression on yeast was measured by binding signal of 5 μg mL⁻¹ anti-cMyc antibody (ca #A21281, Invitrogen) in PBSE (9.6 mM phosphate, 137 mM NaCl, 2.7 mM KCl pH 7.4 and 1 mM EDTA) supplemented with 0.5% BSA (PBSE/BSA) for 30 minutes at 4° C., with shaking. After incubation, cells were washed three times with 100 μL PBSE/BSA and staining with 4 μg mL⁻¹ goat anti-Chicken IgY Alexa Fluor 488 (ca #A11039, Invitrogen) for 30 minutes at 4° C., with shaking. The cells were then washed with PBSE/BSA, and resuspended in 100 μL of PBSE/BSA and analyzed on a HyperCyt screener (Sartorius). Signals reported are median fluorescence intensities. Preferential binding towards the extracellular domain of CD99 was confirmed on yeast by comparing binding signal to 10 nM tetramerized SUMO compared to SUMO-CD99 recombinant proteins.

### Bead-Based Binding Assay of Fab Clones:

The binding titration of the Fab clones was performed on Dynabeads M-280 Streptavidin (ca #11206D, Invitrogen) immobilized with biotinylated antigen, as described previously (Hattori et al., “The ACE2-Binding Interface of SARS-CoV-2 Spike Inherently Deflects Immune Recognition,” J. Mol. Biol. 433:166748 (2021) and Nishikori et al., “Broad Ranges of Affinity and Specificity of Anti-Histone Antibodies Revealed by a Quantitative Peptide Immunoprecipitation Assay,” J. Mol. Biol. 424: 391-399 (2012), which are hereby incorporated by reference in their entirety). Binding of Fab clones to the targets on the beads was analyzed on a HyperCyt screener (Sartorius). Signals reported are median fluorescence intensities.

### Mammalian Cell Culture:

KOPT-K1 cells were a gift of Dr. Leandro Cerchietti (Weill-Cornell, New York, NY), DND-41 cells were a gift from Dr. Ioannis Aifantis (NYU Grossman School of Medicine, New York, NY), MOLM-13 (ca #ACC554) and OCI-AML3 (ca #ACC582) were purchased from Leibniz Institute DSMZ, Jurkat (ca #TIB152), MOLT-3 (ca #CRL1552), and CCRF-SB (ca #CCL120) cells were purchased from ATCC. Human T-ALL specimens were obtained from patients at NYU Langone Health. Healthy human PBMC specimens were purchased from STEMCELL Technologies, ca #70025.1. All cell lines were grown in 5% CO₂ at 37° C. under media conditions described by the vendor or the source laboratory. Free of mycoplasma contamination was confirmed using the e-Myco plus Mycoplasma PCR detection kit (ca #25234, Bulldog Bio).

### Cell-Based Binding Analysis Using Cells Endogenously Expressing CD99:

Cells were confirmed to express CD99 by incubation at 4° C. for 30 minutes with anti-CD99 mouse IgM, HO36-1.1 (ca #ab212605, Abcam) and matched mouse IgM isotype, MM-30 (ca #ab18400, Abcam), then staining with Goat anti-Mouse IgM Cross-Adsorbed Secondary Antibody, DyLight 650 (ca #SA5-10153, Invitrogen) at a 1:100 dilution. Cells were incubated with antibody clones and controls for 30 minutes, washed three times with PBS (2.7 mM KCl, 1.5 mM KH₂PO₄, 138 mM NaCl, 8.1 mM Na₂HPO₄-7H₂0) supplemented with 2% FBS (ca #100106, GeminiBio) (PBS/2% FBS), followed by staining with a secondary antibody. Goat anti-mouse IgG (H+L) conjugated with Dylight 650 (ca #84545, Invitrogen) and goat Anti-Human IgG, F(ab′)2 conjugated with Alexa Fluor 647 (ca #109605097, Jackson ImmunoResearch Inc) were used for detecting the binding of scFv-Fc at a 1:100 dilution, and the human IgG and Fab, respectively. The cells were washed three times, and resuspended in 100 μL of PBS/2% FBS and analyzed on a HyperCyt screener (Satorius). For healthy donor PBMC and T-ALL patient samples cells were stained with anti-human CD45 conjugated with BB515 (ca #BDB564585, BioRad), anti-human CD34 conjugated with BV421 (ca #BDB562577, BioRad), anti-human CD2 conjugated with APC (ca #300214, Biolegend), and anti-human CD7 conjugated with PE/Cy7 (ca #343114, Biolegend) antibodies. Gating for flow cytometry consisted of FSC vs SSC cell gating, followed by single cell gating in FSC-H or FSC-A vs FSC-W to remove aggregates. For all samples cells were gated for Propidium Iodide negative. T-ALL patient samples cells were then gated for human CD45 positive followed by CD34 negative, and finally CD7 positive. For healthy donor PBMC samples cells were gated for human CD45 positive followed by CD34 positive and CD34 negative. Data collection performed on ZE5 Cell Analyzer (BioRad) or Guava easyCyte (Luminex) flow cytometers. Signals reported are median fluorescence intensities.

### Affinity Maturation by Random Mutagenesis:

Random mutagenesis for affinity maturation was performed by error-prone PCR. To increase the mutation rate of Taq DNA polymerase, MnCl₂ was added to the PCR reaction (Cadwell & Joyce, “Mutagenic PCR,” PCR Methods Appl. 3:S136-40 (1994), which is hereby incorporated by reference in its entirety). Briefly, two error prone PCRs using GoTaq Flexi DNA polymerase (Promega) were performed with final concentrations of MnCl₂ of 0.1 and 0.3 mM. The variable heavy chain and light chain were amplified separately, then combined and amplified via overlap PCR. The yeast cells were transformed with the amplified PCR products and the digested vector, and they were assembled via homologous recombination in the yeast cells. Two rounds of FACS were preformed using 100 nM of tetramerized SUMO-CD99, as described above. Preferential binding of the scFv clones to CD99 was assessed as described above, except 5 nM tetramerized targets were used. High affinity clone was identified by comparing binding signal of single clones, using 5 nM tetramerized targets, to the parental clone. Titrations of clones showing enhanced binding to tetramerized CD99 were performed to assess apparent affinity of the scFv displayed on the yeast. Clone 10A1 had the highest affinity to tetramerized CD99 on yeast and it was selected for further antibody characterization.

Production of Fab Antibody Fragment in E. coli:

The Fab fragments of clones were produced by attaching the V_{H} and V_{L} fragments of the scFv to the C_{H1} and C_{L} fragments, respectively, as previously described (Hattori et al., “Recombinant Antibodies to Histone Post-Translational Modifications,” Nat. Methods 10:992-995 (2013), which is hereby incorporated by reference in its entirety). The antibody Fab fragments also contained a C-terminal AviTag (Avidity), on the heavy chain, to facilitate biotinylation using the BirA biotin ligase. They were expressed in E. coli 55244 (ca #55244, sATCC) and purified using a HiTrap Protein G HP column (Cytivia) using 20 mM sodium phosphate buffer (pH 7.0) and eluted with 0.1 M Glycine-HCl (pH 2.7). A portion of the eluted Fab clones were biotinylated in vitro using BirA. All biotinylated and non-biotinylated Fabs were further purified on a Resource S column (Cytivia) using a linear gradient of NaCl in 50 mM sodium acetate buffer (pH 5.0). Purity was assessed by SDS-PAGE.

Production of scFv-Fc and Human IgG1-Based Antibodies:

The scFv-Fc antibodies were produced by attaching the scFv fragment to a mouse IgG1 Fc fragment containing C-terminal AviTag and His tags (Bujak et al., “Reformatting of scFv Antibodies into the scFv-Fc Format and Their Downstream Purification,” Monoclonal Antibodies: Methods and Protocols, 2nd Edition 1131:315-334 (2014); Crothers & Metzger, “The Influence of Polyvalency on the Binding Properties of Antibodies,” Immunochemistry 9:341-357 (1972); Moutel et al., “A Multi-Fc-Species System for Recombinant Antibody Production,” BMC Biotechnol 9:14 (2009); and Suh et al., “Cloning, Genomic Organization, Alternative Transcripts and Expression Analysis of CD99L2, A Novel Paralog of Human CD99, and Identification of Evolutionary Conserved Motifs,” Gene 307:63-76 (2003), each of which are hereby incorporated by reference in their entirety). The human IgG1-based antibodies were produced by attaching the V_{H} and V_{L} fragments to the IgG1 CH₁CH₂CH₃ fragment and C_{L} fragments, respectively. The human antibodies contain LALA-PG mutations: L234A and L235A mutations to eliminate binding to Fc receptors, and P329G to eliminate complement binding (Lo et al., “Effector-Attenuating Substitutions That Maintain Antibody Stability and Reduce Toxicity in Mice,” J. Biol. Chem. 292:3900-3908 (2017), which is hereby incorporated by reference in its entirety). In addition, the ‘inducible’ hexamer antibody contains an E345R mutation to promote IgG hexamerization upon antigen binding and the ‘constitutive’ hexamer antibody contains E345R, E440G, and S440Y mutations that promote the formation of IgG hexamers in solution, in a concentration dependent manner (Diebolder et al., “Complement is activated by IgG hexamers assembled at the cell surface,” Science 343:1260-1263 (2014) and de Jong et al., “A Novel Platform for the Potentiation of Therapeutic Antibodies Based on Antigen-Dependent Formation of IgG Hexamers at the Cell Surface,” PLoS Biol. 14:e1002344 (2016), which are hereby incorproated by reference in their entirety). The tetravalent IgG HCscFv antibody was produced by attaching a GS₆GS₂GS₄ linker connected to the scFv onto the C-terminal heavy chain. The variable heavy chains were cloned into pFUSEss-CHIg plasmid (ca #pfusess-hchg1, Invivogen) containing the mutations described above, the variable light chains were cloned into pFUSEss-CLIg-hk plasmids (ca #pfuse2ss-hclk, Invivogen).

The ExpiCHO-S cells (Gibco) were transiently transfected with the plasmids using the ExpiFectamine CHO Transfection kit, (ca #A29129, Gibco) according to manufacturer's protocol. The cells were incubated at 37° C. with 8% CO₂ and harvested after 3-7 days. The cell culture was clarified by centrifugation and supernatant was supplemented with Complete ultra tablets, mini EDTA Free, (ca #5832731001, Roche) and 1 mM PMSF. The supernatant was filtrated and recombinant proteins were purified. The scFv-Fc antibodies were purified by affinity chromatography using a HisTrap excel column (Cytivia) followed by size exclusion chromatography using a Superdex 200 10/300 column (Cytivia). The human IgG1-based antibodies were purified by affinity chromatography using a Protein A column (ca #PROA102, GORE) followed by cation exchange chromatography using a Resource S column.

### Target Protein Truncations for Epitope Mapping Using Western Blotting:

Kunkel mutagenesis was utilized to make truncated CD99 ECD fragments (23-122, 33-122, and 43-122) and a combination of Kunkel mutagenesis and restriction and insertion cloning were utilized to produce the other CD99 fragments (53-122, 63-122, 73-122, 83-122, 93-122, 103-122, and 113-122) (Kunkel, T. A., “Rapid and Efficient Site-Specific Mutagenesis without Phenotypic Selection,” Proc Natl Acad Sci USA 82:488-492 (1985), which is hereby incorporated by reference in its entirety). The proteins were expressed and purified as described above, except they were only purified using a Ni-Sepharose resin (Cytivia). SDS-PAGE of 50 ng of each purified protein was performed followed by transfer to Immobilon-P^{SQ} PVDF membrane (ca #ISEQ00010, Millipore). For Western blot detection, the membranes were blocked with 5% skim milk overnight at 4° C., then washed three times with 1×TBST (50 mM Tris pH 7.5, 150 mM NaCl, and 0.05% Tween-20). The membranes were then incubated with 14 nM of clones 10, 22, and 30, 1 nM of HO36-1.1, or 1:1000 of mouse anti-His (ca #37-2900, Invitrogen) for 1.5 hours at room temperature, then washed as described above. The membranes were then incubated with goat anti-mouse IgG Fc conjugated with horseradish peroxidase (HRP) (ca #31437, Invitrogen) or goat anti-mouse IgM-HRP (ca #626820, Invitrogen), for 1 hour at room temperature, then washed as described above. Pierce ECL 2 Western Blotting Substrate (ca #80198, Thermo) was added according to manufacturer protocol. Signal detection was analyzed on ChemiDoc Touch imaging system (Biorad).

### Production of SAV-Fab Dimer, Trimer, and Tetramer:

To form streptavidin complexes with two to four functional biotin-binding sites we utilized a streptavidin triple mutant (N23A, S27D, S45A) with a ‘dead’ biotin-binding site (Howarth et al., “A Monovalent Streptavidin with a Single Femtomolar Biotin Binding Site,” Nat. Methods 3:267-273 (2006), which is hereby incorporated by reference in its entirety) and wildtype ‘alive’ streptavidin containing N-terminal 6×His tag. BL21(DE3) cells were transformed, separately, with the ‘dead’ and ‘alive’ SAV expression plasmids. The target proteins were expressed via isopropyl β-D-1-thiogalactopyranoside (IPTG) induction at 37° C. for 18 hours, then purified from the insoluble fraction after resuspension with GuHCl buffer (20 mM TrisHCl, 6 M GuHCl, pH 8.0). The dead and alive SAV were mixed at a 1:1 ratio and refolding was performed by rapid dilution into TBS (50 mM Tris pH 7.5 and 150 mM NaCl). The mixture was then filtered and purified using immobilized metal affinity chromatography using a Ni-Sepharose resin (Cytivia), this allowed for the capture of only A1D3, A2D2, A3D1, and A4 SAV complexes since they contain His-tags, whereas the D4 does not. The SAV complexes with two, three and four functional biotin binding sites were further purified on a Resource S column (Cytivia). A1D3, A2D2, A3D1, and A4 have an increasing number of 6×His tags, one, two, three, and four, respectively. Consequently, the increasing number of 6×His tags make the complexes more positively charged, causing the A1D3, A2D2, A3D1, and A4 to elute from the cation-exchange chromatography, in that order. Once purified the SAV complexes were dialyzed into PBS buffer. To form the SAV-Fab complexes the A2D2, A3D1, and A4 SAV were separately mixed with biotinylated Fab at a ratio of 1:4 then incubated at 4° C. for 30 minutes.

### In Vitro Antibody Cytotoxicity Assay:

For cell line cytotoxicity assays, 25,000 cells per well were treated with antibody or control. For primary sample cytotoxicity assays, 1×10⁶ cells per well were treated with antibody or control. A final volume of 100 μL per well in a 96-well, non-treated, flat-bottom microplates (ca #0877253, Falcon) was incubated in 5% CO₂ at 37° C. for 18 hours. After the 18-hour treatment cells were visualized under a light microscope to assess cell aggregation. Cells were dissociated by incubating with recombinant CD99 at ≥5 μM and Accumax (ca #07921, StemCell) for 15 minutes, then stained with Annexin V 488 (ca #A1320, Life technologies) according to manufacturer's instructions and propidium iodide at a final concentration of 5 μg/mL (ca #P4170-25MG, Sigma) at room temperature for 15 minutes. SureCount standard 3 μm microspheres (ca #CC03N, Bangs Laboratories, Inc) were diluted 1:10 in PBS buffer and added to stained cells to measure cell count. Fluorescence and cell count were analyzed by flow cytometry. For healthy donor PBMC and T-ALL patient samples cells were stained with anti-human CD45 conjugated with BB515 (ca #BDB564585, BioRad), anti-human CD34 conjugated with BV421 (ca #BDB562577, BioRad), and anti-human CD7 conjugated with PE/Cy7 (ca #343114, Biolegend) antibodies. Gating for flow cytometry consisted of FSC vs SSC cell gating, followed by single cell gating in FSC-H or FSC-A vs FSC-W to remove aggregates. T-ALL patient samples were then gated for human CD45 positive followed by CD34 negative, and finally CD7 positive. For healthy donor PBMC samples cells were gated for human CD45 positive followed by CD34 positive and CD34 negative. For all samples gating of Annexin V positive and Propidium Iodide positive was performed to assess percentage of apoptotic cells. Data collection performed on ZE5 Cell Analyzer (BioRad) or Guava easyCyte (Luminex) flow cytometers.

### X-Ray Crystallography and Structural Determination:

The CD99 peptide containing the 10A1 epitope, Ac-GENDDPRPPNPPKPM-amide (SEQ ID NO: 83) (New England Peptide), was mixed with the 10A1 fab at a 2:1 molar ratio of peptide to fab to a final concentration of ˜7 mg/mL. The condition at which crystals formed consisted of 0.2 M lithium sulfate monohydrate, 0.1 M HEPES, pH 7.3, 25% PEG3350, (ca #HR2-144, Hampton Research) and 0.1 M L-Proline (ca #HR2-428, Hampton Research). The crystal used for x-ray diffraction had a low three-dimensional shape and was a large two-dimensional plate. Diffraction data were collected at Advance Photon Source at the Argonne National Laboratory using the 19-ID beam line. Data processing was performed using HKL2000 (Otwinowski & Minor, “Processing of X-ray Diffraction Data Collected in Oscillation Mode,” Methods Enzymol. 276:307-326 (1997), which is hereby incorporated by reference in its entirety) and the starting model was built using SWISS-MODEL (Waterhouse et al., “SWISS-MODEL: Homology Modelling of Protein Structures and Complexes,” Nucleic Acids Res. 46:W296-W303 (2018), which is hereby incorporated by reference in its entirety) and molecular replacement was performed using Phaser (McCoy et al., “Phaser Crystallographic Software,” J. Appl. Crystallogr. 40:658-674 (2007), which is hereby incorporated by reference in its entirety). Phenix refinement (Afonine et al., “Towards Automated Crystallographic Structure Refinement with phenix.refine,” Acta Crystallogr. D. Biol. Crystallogr. 68:352-367 (2012), which is hereby incorporated by reference in its entirety) and Coot (Emsley et al., “Features and Development of Coot,” Acta Crystallogr D. Biol. Crystallogr. 66:486-501 (2010), which is hereby incorporated by reference in its entirety) utilized for refinement and the h, -k, -l, twinning law applied. The ‘Protein interfaces, surfaces and assemblies’ service PISA at the European Bioinformatics Institute, PDBePISA, was used to determine the buried interface (Krissinel, E., “Crystal Contacts as Nature's Docking Solutions,” J. Comput. Chem. 31:133-143 (2010) and Krissinel, E. & Henrick, et al., “Inference of Macromolecular Assemblies from Crystalline State,” J. Mol. Biol. 372:774-797 (2007), which are hereby incorporated by reference in their entirety).

### Example 1—Development of CD99-Binding Human Antibodies

The CD99 extracellular domain (ECD) is rich in glycine, serine, and proline residues and predicted to be disordered (Romero et al., “Sequence Complexity of Disordered Protein,” Proteins 42:38-48 (2001); Williams et al., “The Protein Non-Folding Problem: Amino Acid Determinants of Intrinsic Order and Disorder,” Pac. Symp. Biocomput. 89-100 (2001); and Vucetic et al., “Flavors of Protein Disorder,” Proteins 52:573-584 (2003), which are hereby incorporated by reference in their entirety). The CD99 ECD was produced as SUMO- and GFP-fusion proteins to facilitate production and purification. A human naïve antibody library was screened in the single-chain variable fragment (scFv) format using yeast surface display technology (Feldhaus et al., “Flow-Cytometric Isolation of Human Antibodies from a Nonimmune Saccharomyces cerevisiae Surface Display Library,” Nat. Biotechnol. 21:163-170 (2003) and Chao et al., “Isolating and Engineering Human Antibodies using Yeast Surface Display,” Nat. Protoc. 1:755-768 (2006), which are hereby incorporated by reference in their entirety). Because antibodies to a disordered antigen tend to have low affinity, library sorting was performed using tetramerized CD99 ECD by coupling the biotinylated antigen to streptavidin in order to enhance the binding by the avidity effect (Chao et al., “Isolating and Engineering Human Antibodies using Yeast Surface Display,” Nat. Protoc. 1:755-768 (2006) and Altman et al., “Phenotypic Analysis of Antigen-Specific T Lymphocytes,” Science 274:94-96 (1996), which are hereby incorporated by reference in their entirety). Three clones named 10, 22, and 30 were identified.

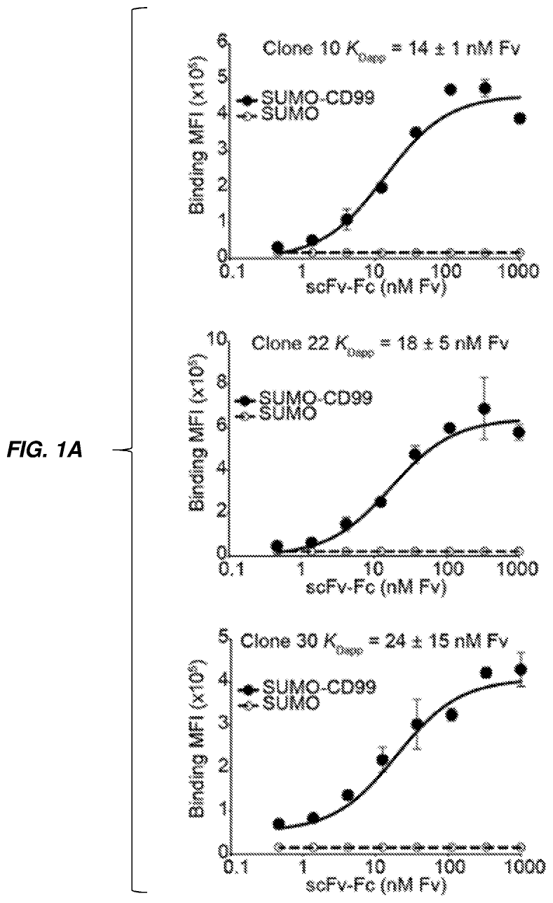

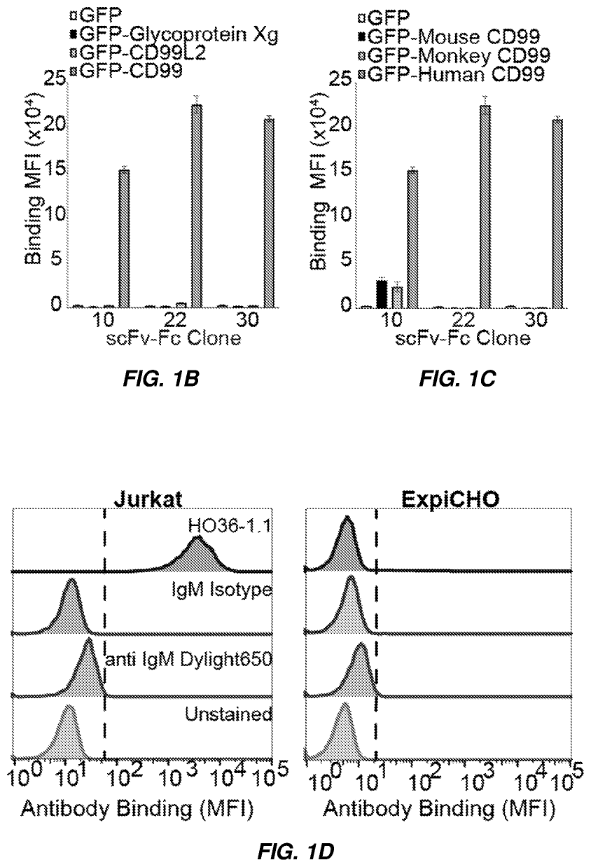

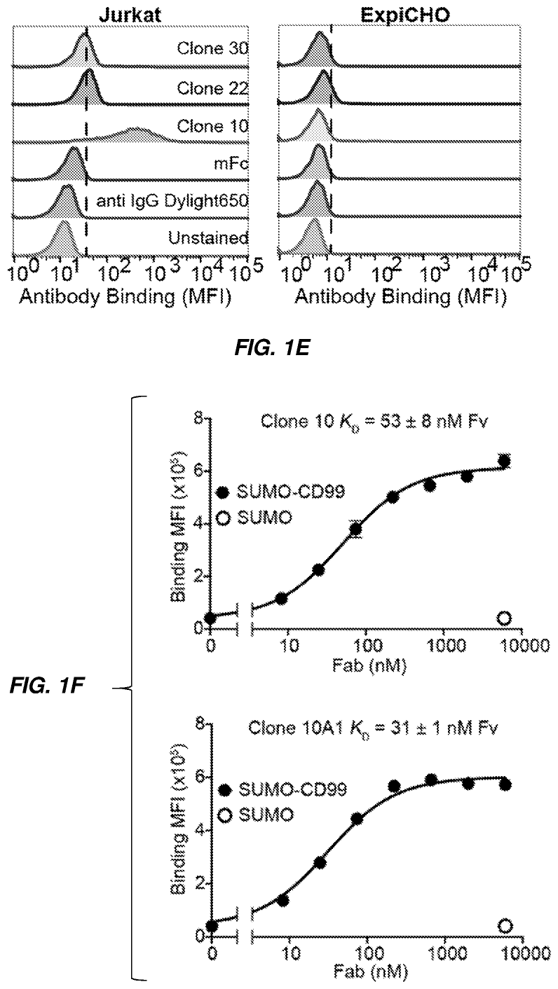

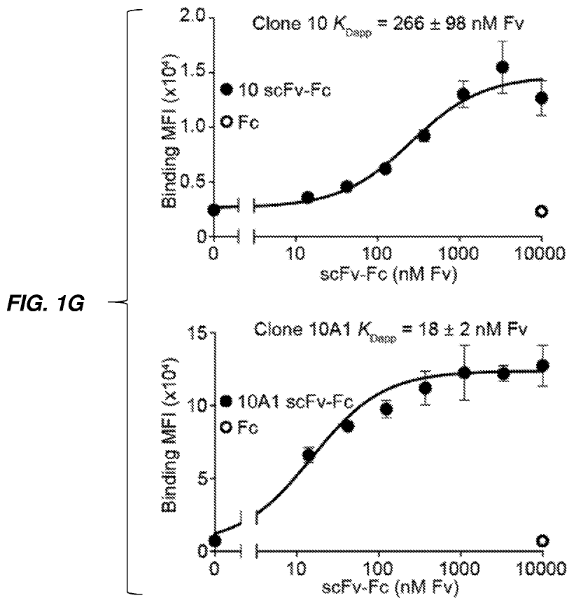

To facilitate rapid characterization of these antibody clones, they were produced in a scFv-Fc format that mimics the bivalent IgG architecture, but does not require the production of separate heavy and light chains (Bujak et al., “Reformatting of scFv Antibodies into the scFv-Fc Format and Their Downstream Purification,” Monoclonal Antibodies: Methods and Protocols, 2nd Edition 1131:315-334 (2014); Crothers & Metzger, “The Influence of Polyvalency on the Binding Properties of Antibodies,” Immunochemistry 9:341-357 (1972); Moutel et al., “A Multi-Fc-Species System for Recombinant Antibody Production,” BMC Biotechnol 9:14 (2009); and Suh et al., “Cloning, Genomic Organization, Alternative Transcripts and Expression Analysis of CD99L2, A Novel Paralog of Human CD99, and Identification of Evolutionary Conserved Motifs,” Gene 307:63-76 (2003), which are hereby incorporated by reference in their entirety). These antibodies bound the immobilized CD99 ECD with apparent K_{D} values ≤24 nM Fv (FIG. 1A); K_{D} values are expressed in terms of nM Fv to minimize ambiguity across different antibody formats throughout this paper. To assess the specificity of the antibodies, their cross-reactivity to CD99 homologs was examined.

CD99 is a member of the CD99 family and has two homologs, CD99L2 and Glycoprotein Xg, with their ECDs showing relatively low homology, with 35% and 34% sequence identity to that of CD99, respectively (Suh et al., “Cloning, Genomic Organization, Alternative Transcripts and Expression Analysis of CD99L2, A Novel Paralog of Human CD99, and Identification of Evolutionary Conserved Motifs,” Gene 307:63-76 (2003) and Ellis et al., “Pbdx Is the Xg Blood-Group Gene,” Nature Genetics 8:285-290 (1994), which are hereby incorporated by reference in their entirety). The three antibodies bound to the CD99 ECD, but not to those of CD99L2 or Glycoprotein Xg (FIG. 1). Next, cross-reactivity to CD99 orthologs was assessed. Mouse and monkey CD99 exhibit 44% and 90% sequence identity to human ECD of CD99, respectively. Clone 10 cross-reacted with mouse and monkey CD99, but clones 22 and 30 did not, suggesting the epitopes of the latter antibodies are in regions that substantially differ between human and monkey CD99 (FIG. 1C).

CD99 is a 32 kDa O-linked glycosylated transmembrane protein, with almost half the mass, 14 kDa, corresponding to the sugar molecules (Aubrit et al., “The Biochemical 15 Characterization of E2, a T Cell Surface Molecule Involved in Rosettes,” Eur. J. Immunol. 19:1431-1436 (1989), which is hereby incorporated by reference in its entirety). To assess if the antibodies bind to CD99 with posttranslational modifications, their binding to CD99 on cells was tested. First, CD99 expression on Jurkat cells was confirmed using a commercially available anti-CD99 IgM antibody, clone HO36-1.1 (FIG. 1D). All three antibodies detectably bound to Jurkat cells, and clone 10 bound more efficiently than clones 22 and 30 (FIG. 1E). In contrast, none bound to CHO cells, hamster cells that do not express human CD99 (FIG. 1D, E).

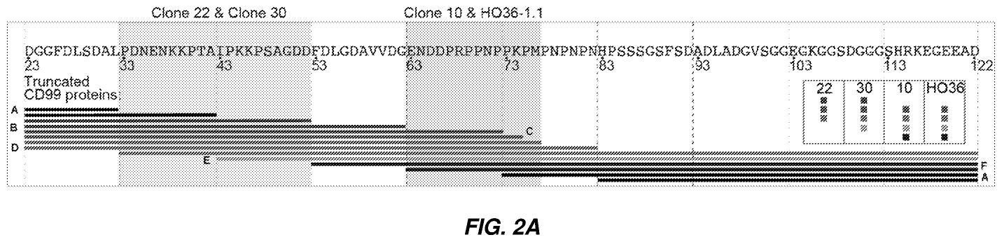

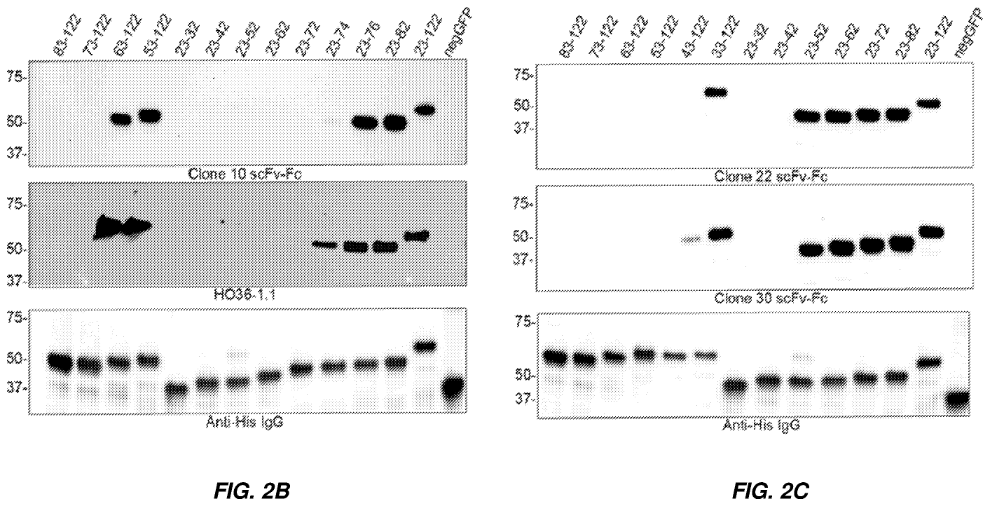

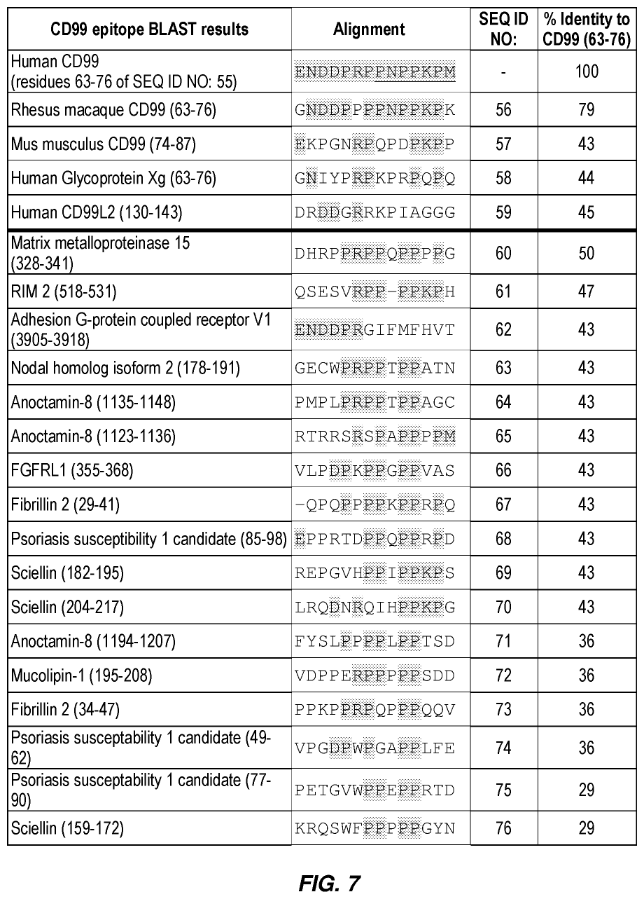

Because the ECD of CD99 is predicted to be disordered, it was predicted that the antibodies bind to a linear epitope. To test this hypothesis, Western blots of recombinant CD99 was performed with the antibodies. Each of the antibodies indeed produced strong bands in Western blotting (FIG. 2). To further delineate the epitopes, truncated forms of the CD99 ECD were produced, and antibody binding was tested by Western blotting. Clone 10 bound to four different fragments, allowing Applicant to deduce that its epitope encompassed amino acid residues 63 to 76 (FIG. 2A-B). Met76 is one of three residues within the epitope (residues 63-76) that differs between human and rhesus CD99 (FIG. 7), which indicates that residue 76 is important in the interaction between clone 10 and CD99. Similar analysis determined that clone 22 bound to residues between 33 and 52 (FIG. 2C) and clone 30 bound to residues between 43 and 52 (FIG. 2C). The overlapping epitopes for clones 22 and 30 include Thr41, a known 0-glycosylation site (Sun et al., “Evolutionarily Conserved Paired Immunoglobulin-Like Receptor Alpha (PILRalpha) Domain Mediates its Interaction with Diverse Sialylated Ligands,” J. Biol. Chem. 287:15837-15850 (2012), which is hereby incorporated by reference in its entirety), whereas the epitope for clone 10 includes no known or predicted post-translational modification sites. These results and the fact that we used aglycosylated antigens for antibody discovery suggest that glycosylation at Thr41 weakened binding of clones 22 and 30 to Jurkat cells. It was also determined that the epitope of HO36-1.1, a mouse IgM antibody with cytotoxic function in T-ALL and AML (Pettersen et al., “CD99 Signals Caspase-Independent T Cell Death,” J. Immunol. 166:4931-4942 (2001) and Chung et al., “CD99 is a Therapeutic Target on Disease Stem Cells in Myeloid Malignancies,” Sci. Transl. Med. 9(374):eaaj2025 (2017), which are hereby incorporated by reference in their entirety), overlaps with that for clone 10 (FIGS. 2A-B). Ultimately, clone 10 was chosen for further characterization, because it exhibited the strongest binding to endogenously expressed CD99 and it binds to an epitope that overlaps with that of the cytotoxic IgM antibody.

To identify possible off-targets of clone 10, a BLAST search of its epitope, residues 63 to 76, was performed and 17 segments from ten proteins that are expressed extracellularly were found (FIG. 7). These segments are predominantly proline-rich sequences, but all have ≤50% sequence identity to the clone 10 epitope. Binding of clone 10 to monkey and mouse CD99, with 79% and 43% sequence identity, respectively, was significantly diminished (FIGS. 1C, 6). Together, these data indicate that off-target binding is unlikely, as clone 10 only weakly bound to monkey CD99 that exhibits the highest sequence identity to human CD99.

Next, clone 10 was produced in the Fab format and its binding to CD99 immobilized on streptavidin beads was measured (Hattori et al., “The ACE2-Binding Interface of SARS-CoV-2 Spike Inherently Deflects Immune Recognition,” J. Mol. Biol. 433:166748 (2021) and Nishikori et al., “Broad Ranges of Affinity and Specificity of Anti-Histone Antibodies Revealed by a Quantitative Peptide Immunoprecipitation Assay,” J. Mol. Biol. 424: 391-399 (2012), which are hereby incorporated by reference in their entirety); its apparent K_{D} was determined to be 53±8 nM Fv. To enhance the affinity, a random mutagenesis of the heavy and light chain variable regions was performed and a clone with enhanced binding, termed 10A1, with apparent K_{D}=31 ±1 nM Fv was identified (FIG. 1F). Interestingly, 10A1 in the scFv-Fc format exhibited greater enhancement of binding to endogenously expressed CD99 on Jurkat cells, with apparent K_{D}=18 ±2 nM Fv, a fourteen-fold improvement over that of Clone 10 (266±98 nM Fv) (FIG. 1G).

### Example 2—Valency of Two is Insufficient for Inducing Cytotoxicity

Next, whether clone 10 and 10A1 elicit cytotoxicity in T-ALL cells was examined. Jurkat cells were treated with 5 μM of Clone 10 scFv-Fc antibody, a concentration 37 times greater than the apparent K_{D} for cell-based affinity, and no effects in increasing apoptosis after 24 hours were found. Similarly, no significant changes in cell viability or apoptosis with 5 μM 10A1 scFv-Fc were detected. These results stand in stark contrast to the ability of HO36-1.1 to induce cytotoxicity in AML and T-ALL cells, particularly because all these antibodies bind to overlapping epitopes and HO36-1.1 has a lower apparent affinity to the recombinant ECD of CD99 (226±147 nM), compared with clones 10 and 10A1 (53±8 and 31±1 nM, respectively; FIG. 1F). These results suggest that affinity to CD99 is not a major determinant of the difference in cytotoxicity between the antibodies disclosed herein and HO36-1.1. Clone 10 and 10A1 in the scFv-Fc format are bivalent, whereas HO36-1.1 in the IgM format is decavalent, suggesting that a higher valency of a CD99-binding antibody may be crucial for inducing cytotoxicity.

### Example 3—Higher Valency Enhances Leukemia Cell Cytotoxicity but not Cell Surface Binding In Vitro

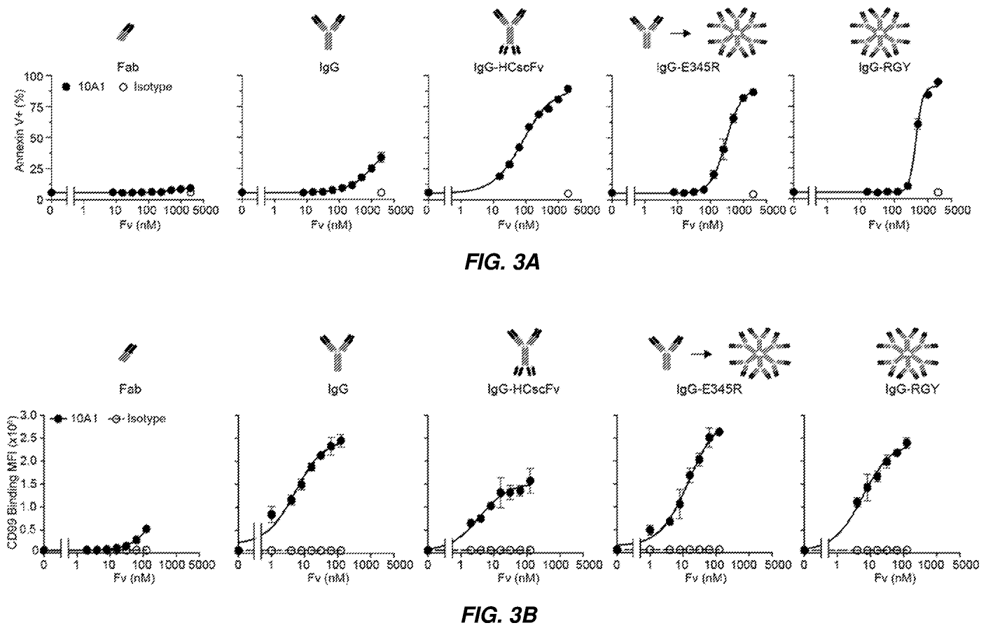

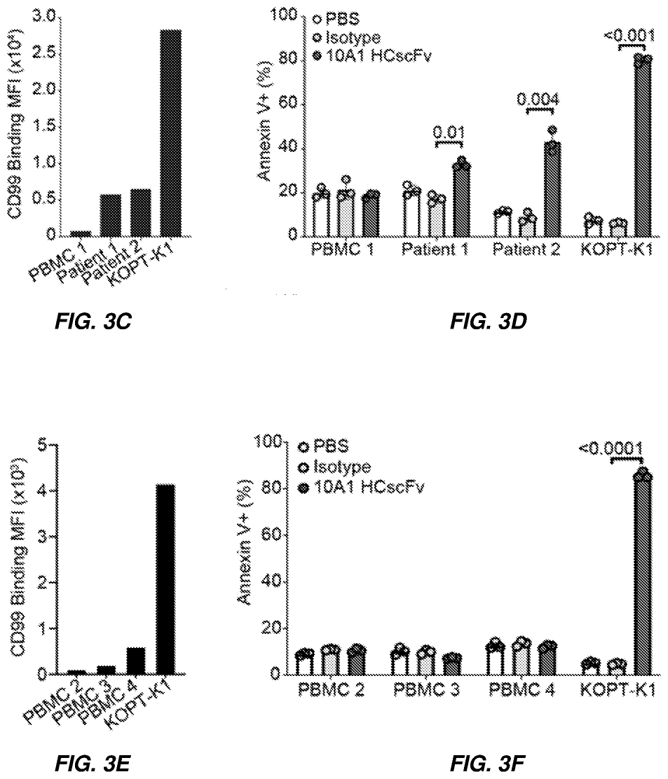

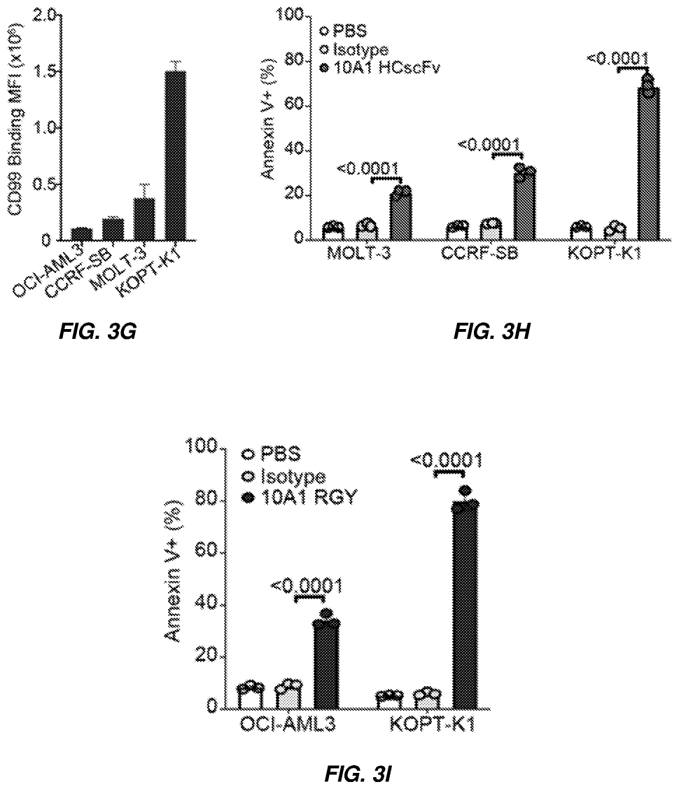

To examine if a valency greater than two is required for inducing cytotoxicity four antibodies containing the same VL and VH domains were generated from 10A1: IgG with a valency of two, IgG-HCscFv with a valency of four, and IgG-E345R ‘inducible hexamer’ and IgG-RGY ‘constitutive hexamer’ with a valency of twelve (FIG. 3A) (Diebolder et al., “Complement is activated by IgG hexamers assembled at the cell surface,” Science 343:1260-1263 (2014) and de Jong et al., “A Novel Platform for the Potentiation of Therapeutic Antibodies Based on Antigen-Dependent Formation of IgG Hexamers at the Cell Surface,” PLoS Biol. 14:e1002344 (2016), which are hereby incorporated by reference in their entirety). When incubating KOPT-K1 cells, a human T-ALL cell line, with the antibodies it was observed that those with a valency of two or more induced homotypic cell aggregation, a phenomenon previously seen when incubating Jurkat or CD4⁺CD8⁺ T cells with antibodies against CD99 (Kasinrerk et al., “CD99 Monoclonal Antibody Induce Homotypic Adhesion of Jurkat cells through Protein Tyrosine Kinase and Protein Kinase C-Dependent Pathway,” Immunol. Lett. 71:33-41. (2000); Bernard et al., “Specifically Triggers Homotypic Aggregation of CD4+CD8+ Thymocytes,” J. Immunol. 154:26-32 (1995); and Khunkaewla et al., “Production, Characterization, and Functional Analysis of Newly Established CD99 Monoclonal Antibodies MT99/1 and MT99/2,” Hybridoma (Larchmt) 26:241-250. (2007), which are hereby incorporated by reference in their entirety). Cell aggregation can lead to inaccurate measures of cell concentration and Annexin V positive cell percentage, because aggregated cells are counted as one in flow cytometry. Thus, a method to disaggregate these cells was sought out. EDTA and DNaseJ, reagents known to promote cell dissociation (Moiseeva et al., “CADM1 Controls Actin Cytoskeleton Assembly and Regulates Extracellular Matrix Adhesion in Human Mast Cells,” PLoS One 9:e85980 (2014) and Price, P. A., “The Essential Role of Ca2+ in the Activity of Bovine Pancreatic Deoxyribonuclease,” J. Biol. Chem. 250:1981-1986 (1975), which are hereby incorporated by reference in their entirety), did not dissociate the cells. In contrast, an addition of excess recombinant CD99 ECD caused the cells to dissociate, likely through inhibition of antibody-CD99 interactions. Consequently, excess CD99 ECD was added to antibody-treated cells prior to analyzing them with a flow cytometry.

Cytotoxicity of the 10A1 antibodies was tested in different formats using KOPT-K1 cells. A dose-dependent increase in the apoptotic cell percentage (FIG. 3A) and a concurrent reduction in the viable cell percentage was detected for the antibodies with a valency greater than two. The IgG format shows only marginal levels of cytotoxicity even at the highest concentrations. The tetravalent antibody demonstrated the highest efficacy, with the rank order of IgG-HCscFv>IgG-E345R>IgG-RGY, as the IC₅₀ values in terms of the antigen-binding site concentration were 82, 299, and 442 nM Fv, respectively (FIG. 6). It was also found that the 10A1 HCscFv was more effective at inducing cytotoxicity than HO36-1.1. These data indicate that a valency of four is sufficient for inducing T-ALL cytotoxicity.

To assess if enhanced cytotoxic effect is the consequence of enhanced binding to cells, binding of these antibodies was measured to KOPT-K1 cells (FIG. 3B). Contrary to Applicant's expectation, the apparent K_{D} value of the bivalent antibody was in the low nanomolar range, close to the higher valency antibody formats (FIG. 6). The apparent K_{D} values were similar among IgG-RGY, IgG-HCscFv and IgG (5-6 nM) except that IgG-E345R showed about 3-fold higher K_{D} value (i.e., lower affinity) than those antibodies. Thus, the lack of potent cytotoxic function in the IgG format is not because it had a lower affinity to the cells.

It was observed that the maximum fluorescence intensities of bivalent and dodecavalent antibodies were similar but that of the tetravalent antibody was about a half as much (FIG. 3B). Because all these antibodies share the same Fab portion and they were detected using anti-Fab secondary antibodies, these results suggest that the anti-Fab secondary antibody does not effectively bind to the scFv portion of the tetravalent antibody and that approximately twice as many copies of the bivalent antibody bind to the cells as the tetravalent antibody. This interpretation in turn suggests that all the CD99-binding sites on the tetravalent antibody are engaged with CD99 on the cell surface.

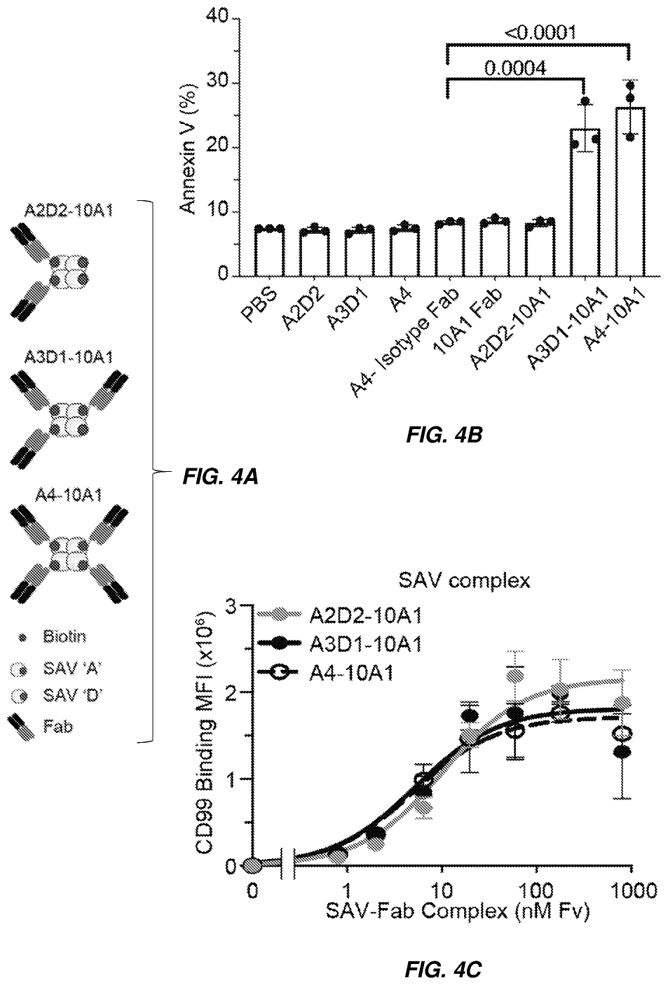

Among the three formats with potent cytotoxicity, the IgG-RGY antibody has the highest affinity to KOPT-K1 cells, but the lowest cytotoxic effect. The size exclusion chromatography data showed that the RGY hexamer dissociated into IgG monomers below 4800 nM Fv, with 50% hexamer at 1895 nM Fv, and 5% at 500 nM Fv, which is consistent with a previous report that the IgG-RGY antibody forms a hexamer in a concentration dependent manner (Wang et al., “Molecular Basis of Assembly and Activation of Complement Component C1 in Complex with Immunoglobulin G1 and Antigen,” Mol. Cell 63:135-145 (2016), which is hereby incorporated by reference in its entirety). These results explain the loss of its cytotoxic activity at concentrations below 500 nM Fv, because the antibody is likely to be in the monomeric IgG format, i.e., bivalent, at these concentrations (FIG. 4A). The concomitant loss of cytotoxic activity and the loss of a high valency structure further supports the view that an antibody needs to engage more than two CD99 molecules to elicit T-ALL cytotoxicity.

### Example 4-10A1 HCscFv is Effective Against Patient-Derived T-ALL Cells and Nontoxic to Healthy Cells

To further investigate the efficacy of 10A1 HCscFv as a potential T-ALL therapy, T-ALL patient samples were treated with 10A1 HCscFv. The patient samples expressed CD99 at a lower level compared with the KOPT-K1 cell line (FIG. 3C). Strikingly, the 10A1 HCscFv antibody increased leukemia cell apoptosis, indicating its effectiveness to T-ALL patient samples (FIG. 3D). Healthy patient peripheral blood mononuclear cells (PBMCs), which expressed CD99 at a lower level than KOPT-K1 cells (FIGS. 3C 3,E), was also treated with 10A1 HCscFv. After treatment, no cytotoxicity was observed, indicating that the antibody does not lead to on-target, off-cancer toxicities in healthy cells (FIGS. 3D, 3F).

Next, 10A1 HCscFv was tested against different cell lines that displayed lower levels of CD99 compared with KOPT-K1 cells, including DND-41 and MOLT-3 (T-ALL cell lines), OCI-AML3 and MOLM13 (AML cell lines), and CCRF-SB (B-ALL cell line) (FIG. 3G). The antibody induced cell death in the T-ALL and B-ALL cell lines, less efficiently than KOPT-K1 cells (FIG. 3H). In contrast, no effects were observed in AML cell lines, which expressed lower levels of CD99 than KOPT-K1 and DND-41. Because the levels of CD99 are very low in OCI-AML3, it was hypothesized that an antibody in a larger size and a higher valency is more effective at ligating more than two CD99 molecules that may be separated with a greater distance. Treatment of OCI-AML3 and KOPT-K1 cells with 10A1 RGY, at high concentration to ensure forming a hexamer, for 18 hours induced apoptosis in both AML and T-ALL cell lines (FIG. 3I). These results indicate that the level of 10A1 cytotoxicity can be fine-tuned toward different levels of cell-surface CD99 by utilizing different antibody formats.

CD99 is expressed on T-ALL blasts both at diagnosis and at relapse. Although CD99 is not required for T-ALL engraftment or self-renewal in an NSG xenograft model, CD99-negative T-ALL cells turn CD99 positive over time, and ultimately all engrafted cells become CD99 positive regardless of their initial surface display levels (Cox et al., “Investigating CD99 Expression in Leukemia Propagating Cells in Childhood T Cell Acute Lymphoblastic Leukemia,” PLoS One 11:e0165210 (2016), which is hereby incorporated by reference in its entirety). These observations may suggest that T-ALL cells could modulate CD99 expression and thereby escape from anti-CD99 therapy. To determine if treatment with the 10A1 tetravalent antibody affects CD99 surface display levels, KOPT-K1 cells were treated with a single dose and escalating doses, and CD99 surface expression was measured. There were no changes in CD99 surface expression, indicating that blasts do not readily escape the treatment by downregulating CD99 expression on the cell surface.

### Example 5—a Valency of Three is Required and Sufficient for Inducing T-ALL Cytotoxicity

To conclusively investigate the minimum valency required for inducing cytotoxicity, streptavidin (SAV)-Fab complexes were generated with a valency ranging from two to four. A streptavidin mutant with a ‘dead’ biotin-binding site was utilized (Howarth et al., “A Monovalent Streptavidin with a Single Femtomolar Biotin Binding Site,” Nat. Methods 3:267-273 (2006), which is hereby incorporated by reference in its entirety). SAV tetramers were formed containing two, three, or four functional subunits, i.e., tetramers comprising A2D2, A3D1 and A4 subunits, respectively, where A and D denote wild-type (alive) and mutant (dead) subunits. Next, biotinylated 10A1 Fab was conjugated to these SAV variants to yield bi-, tri-, and tetra-valent molecules (FIG. 4A).

KOPT-K1 cells were treated for 18 hours, at concentrations more than seventeen times the apparent affinity values of the SAV-10A1 Fab complexes (FIG. 4B-C). Similar to the 10A1 IgG formats, homotypic cell aggregation was observed following incubation with the bi-, tri-, and tetravalent SAV-10A1 Fab, which was readily dissociated using excess CD99 antigen. Cytotoxic effects were detected with the trivalent and tetravalent 10A1 molecules, but not with the monovalent or bivalent molecules (FIG. 4B). The apparent K_{D} values of these molecules were all in the low nanomolar range (FIGS. 4C, 6), similar to the 10A1 IgG formats, confirming the finding that cytotoxicity is not directly dependent on the effective affinity to the cell (FIG. 6). The Fab portion is linked to the SAV via a flexible linker in these constructs, and the efficacy of the tri- and tetra-valent constructs indicate that the close association of two Fab portions in the IgG format is not essential for cytotoxicity. Taken together, these data reveal that a valency of three is necessary and sufficient for inducing T-ALL cytotoxicity.

### Example 6—X-Ray Crystal Structure of 10A1 Fab in Complex with a CD99 Peptide

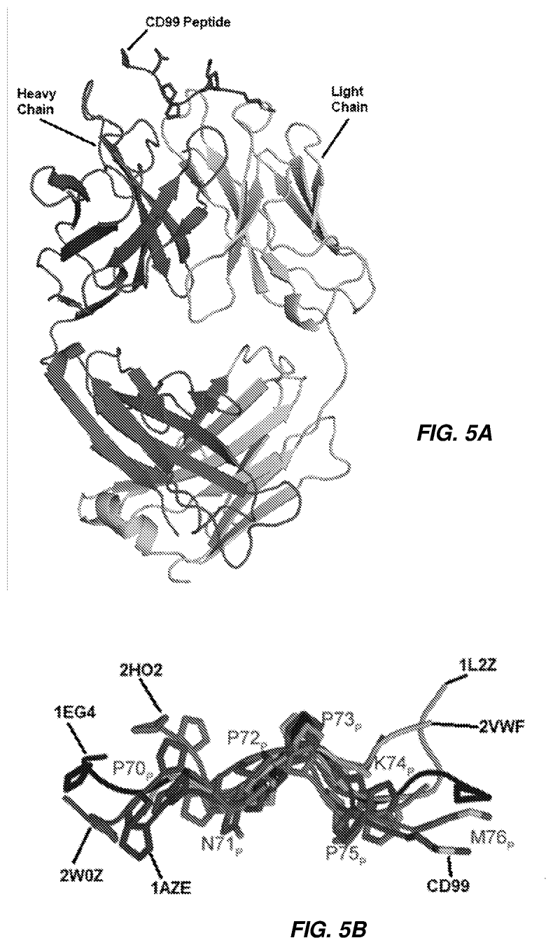

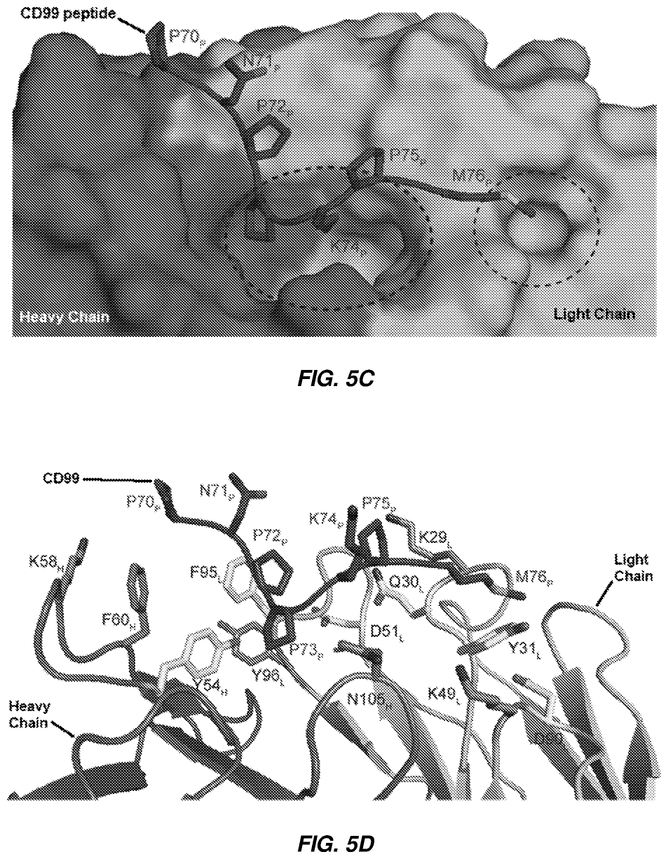

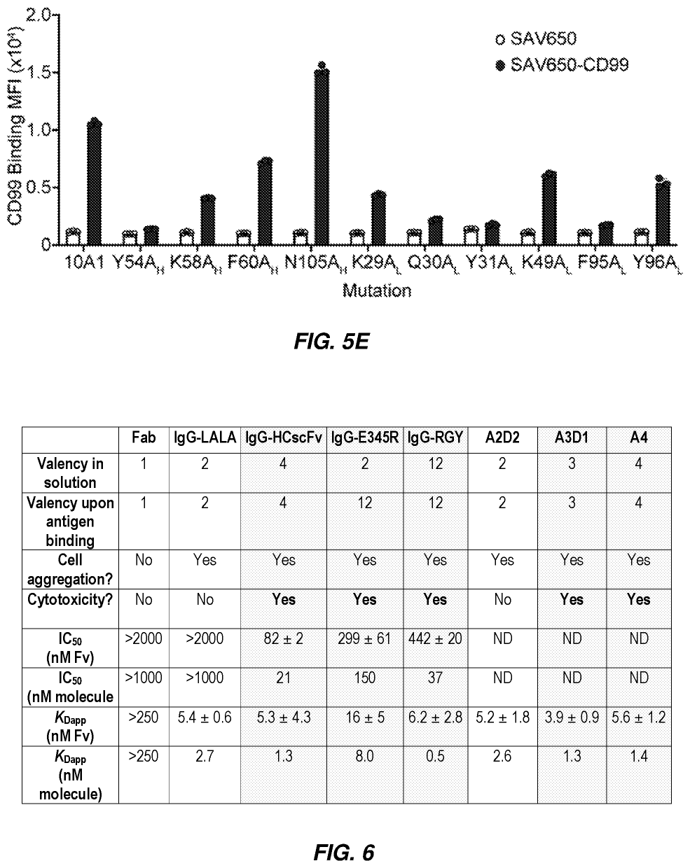

To determine the structural basis for the recognition of CD99 by 10A1 Fab, the crystal structure of 10A1 Fab was determined in complex with a CD99 peptide containing the 10A1 epitope (residues 62-76; FIG. 2) at a resolution of 3.10 Å (FIG. 5A). As expected, 10A1 takes on the canonical global fold of Fab. Of the 15 residues of the CD99 peptides, electron density was observed for PNPPKPM (residues 70-76 of SEQ ID NO: 55), but not for the N-terminal region of the peptide. The observed peptide segment formed a left-handed helix, showing striking similarity to other proline-rich motifs (PRMs) that also form left-handed poly-proline type-II (PPII) helices (FIG. 5B) (Freund et al., “Proline-rich Sequence Recognition Domains (PRD): Ligands, Function and Inhibition,” Handb Exp Pharmacol, 407-29 (2008), which is hereby incorporated by reference in its entirety). PRMs are known ligands for Src homology (SH) 3, WW, Enabled VASP Homology 1 (EVH1) domains as well as CD2 binding protein (CD2BP2) and profiling (Kay et al., “The Importance of Being Proline: The Interaction of Proline-rich Motifs in Signaling Proteins With Their Cognate Domains,” FASEB J. 14:231-41 (2000), which is hereby incorporated by reference in its entirety). However, these well-documented interactions occur predominantly among intracellular proteins. There are precedents of proline rich motifs in extracellular domains of proteins. One of these proteins, mucolipin-1, contains proline rich regions (FIG. 7) that forms a PPII helix (Schmiege et al., “Human TRPML1 Channel Structures in Open and Closed Conformations,” Nature 550:366-370 (2017), which is hereby incorporated by reference in its entirety).

The peptide sits in a cleft at the junction between the heavy and light chains (FIG. 5C). The interface buries 444 Å² of the solvent accessible surface of the peptide. The interface is mainly composed of Van der Waals interactions, with the exception of one possible hydrogen bond between the CD99 peptide residue M11_{P} and Y31_{L} (Krissinel, “Crystal Contacts as Nature's Docking Solutions,” J Comput Chem 31:133-43 (2010); Krissinel et al., “Inference of Macromolecular Assemblies from Crystalline State,” J Mol Biol 372:774-97 (2007), each of which is hereby incorporated by reference in its entirety). Residues of 10A1 that directly made contacts with CD99 were identified as: K54 of the heavy chain, and Q30, Y31, and F95 of the light chain (FIG. 5D). Mutation to alanine of these residues substantially reduced CD99 binding (FIG. 5E). In addition, mutating D50_{L} and D91_{L} in CDR-L2 and -L3, respectively, to serine abolished binding (FIG. 5D), indicating these residues play important roles in the binding to CD99, although they do not directly contact the CD99 peptide in the crystal structure (FIG. 5D). D91_{L} is adjacent to residue V90_{L}, which was one of the residues mutated during affinity maturation, indicating that the A90V mutation contributes to the affinity increase by improving the position of D91_{L}. Mutation analysis also revealed six residues located immediately outside the paratope that show marginal effects upon Ala substitution (FIG. 5E). Altogether, these mutation results validate the crystal structure.

A similarity between the CD99 epitope and PRMs bound to modular domains was observed, and their interface characteristics were compared (FIG. 5B). Although the CD99 peptide structure resembles that of the other PRMs and buries surface areas comparable to these complexes, the affinity of 10A1 to CD99 is much higher than those for the natural ligands of the modular interaction domains (micromolar K_{D} values)(Li, “Specificity and Versatility of SH3 and Other Proline-Recognition Domains: Structural Basis and Implications for Cellular Signal Transduction,” Biochem J 390:641-53 (2005), each of which is hereby incorporated by reference in its entirety). These results demonstrate the feasibility of engineering high-affinity interfaces for RPMs.

### Discussion of Examples 1-6

The preceding Examples provide support for using anti-CD99 antibodies as a therapy in T-ALL. There are currently no FDA approved monoclonal antibodies approved for use in T-ALL (Terwilliger et al., “Acute Lymphoblastic Leukemia: A Comprehensive Review and 2017 Update,” Blood Cancer J7:e577 (2017), which is hereby incorporated by reference in its entirety). This is unfortunate, because the current chemotherapeutic treatment options for T-ALL are not selective and lead to patient toxicities (Hurwitz et al., “Substituting Dexamethasone for Prednisone Complicates Remission Induction in Children with Acute Lymphoblastic Leukemia,” Cancer 88:1964-1969 (2000); Real et al., “Gamma-Secretase Inhibitors Reverse Glucocorticoid Resistance in T Cell Acute Lymphoblastic Leukemia,” Nat. Med. 15:50-58 (2009), each of which is hereby incorporated by reference in its entirety). Even after intensive chemotherapy and radiation, some T-ALL patients relapse or become refractory. The best chemotherapeutic option for relapse/refractory T-ALL patients is nelarabine, but it is associated with a high risk of neurotoxicity (DeAngelo et al., “Nelarabine Induces Complete Remissions in Adults with Relapsed or Refractory T-lineage Acute Lymphoblastic Leukemia or Lymphoblastic Lymphoma: Cancer and Leukemia Group B Study 19801,” Blood 109:5136-42 (2007), which is hereby incorporated by reference in its entirety). Alternative treatment options, such as stem cell transplants, are available for high-risk patients, but toxicity also plays a role in the lack of efficacy, with transplantation-related mortality in older patients (Goldstone et al., “In Adults with Standard-Risk Acute Lymphoblastic Leukemia, the Greatest Benefit Is Achieved from a Matched Sibling Allogeneic Transplantation in First Complete Remission, and an Autologous Transplantation Is Less Effective than Conventional Consolidation/Maintenance Chemotherapy in All Patients: Final Results of the International ALL Trial (MRC UKALL XII/ECOG E2993). Blood 111:1827-33 (2008), which is hereby incorporated by reference in its entirety). There is clearly an unmet need for more effective and less toxic therapies in T-ALL.

The tetravalent antibody described above, 10A1 HCscFv, has multiple favorable attributes that make it a promising candidate for further therapeutic development. It is a fully human antibody in a manufacturable format (Kundranda et al., “Randomized, Double-Blind, Placebo-Controlled Phase II Study of Istiratumab (MM-141) Plus Nab-Paclitaxel and gemcitabine Versus Nab-Paclitaxel and Gemcitabine in Front-Line Metastatic Pancreatic Cancer (CARRIE),” Ann Oncol 31:79-87 (2020); Lee et al., “Phase 1a Study Results Investigating the Safety and Preliminary Efficacy of ABL001 (NOVI501), a Bispecific Antibody Targeting VEGF and DLL4 in Metastatic Gastrointestinal (GI) Cancer,” J Clin Oncol. 37(15 Suppl):3023 (2019), each of which is hereby incorporated). It is selective to human CD99 (FIGS. 1, 7), directly elicits cytotoxicity in T-ALL cells independent of effector-mediated function, such as antibody-dependent cellular cytotoxicity, and it is non-toxic to healthy cells (FIG. 3). It has weak, but substantial, cross-reactivity to monkey and mouse CD99, which can be enhanced via protein-engineering approaches for enabling preclinical studies. The structure of the 10A1-CD99 peptide complex will help improve the cross-reactivity and facilitate the development of variants that bind better to monkey CD99. The epitope includes PNPPKPM (residues 70-76 of SEQ ID NO: 55) and a truncation of PM abrogates binding (FIG. 2). Monkey CD99 has a PNPPKPK motif (see FIG. 7, SEQ ID NO: 56), and the pocket for Met can be altered in such a way that it better accommodates Lys in monkey CD99. In contrast, Mouse CD99 has a distinct QPDPKPP motif (see FIG. 7, SEQ ID NO: 57), and substantial engineering of 10A1 may be required to achieve high-affinity binding to mouse CD99.

The T-ALL cell lines utilized in the preceding Examples contain an activating mutation in the NOTCH1 signaling pathway (Weng et al., “Activating Mutations of NOTCH1 in Human T Cell Acute Lymphoblastic Leukemia,” Science 306:269-71 (2004), which is hereby incorporated by reference in its entirety). This pathway is a major oncogenic driver in T-ALL, with mutations in ≥50% of T-ALL patients (Weng et al., “Activating Mutations of NOTCH1 in Human T Cell Acute Lymphoblastic Leukemia,” Science 306:269-71 (2004); Garcia-Peydro et al., “The NOTCH1/CD44 Axis Drives Pathogenesis in a T Cell Acute Lymphoblastic Leukemia Model,” J Clin Invest 128:2802-2818 (2018); Kimura et al., “NOTCH1 Pathway Activating Mutations and Clonal Evolution in Pediatric T-Cell Acute Lymphoblastic Leukemia,” Cancer Sci 110:784-794 (2019), each of which is hereby incorporated by reference in its entirety). 7-Secretase inhibitors (GSI), which inhibit the Notch signaling pathway, are being evaluated in combination with chemotherapy. Clinical Trial NCT02518113 (completed 2019) evaluated the safety of LY3039478 in combination with dexamethasone in participants with T-cell acute lymphoblastic leukemia or T-cell lymphoblastic lymphoma; and Clinical Trial NCT01363817 (completed 2019) evaluated the safety of BMS-906024, either alone or in combination with Dexamethasone in subjects with T-cell acute lymphoblastic leukemia or T-cell lymphoblastic lymphoma who no longer respond to, or have relapsed from, standard therapies. Interestingly, DND-41 and KOPT-K1 cell lines, but not MOLT-3, are GSI-sensitive (Palomero et al., “Mutational Loss of PTEN Induces Resistance to NOTCH1 Inhibition in T-cell Leukemia,” Nat Med 13:1203-10 (2007), which is hereby incorporated by reference in its entirety). All of these T-ALL cell lines responded to the 10A1 HCscFv treatment (FIG. 3A), indicating that the antibody is potentially efficacious to T-ALL with diverse driver mutations.

It was observed that 10A1-mediated cytotoxicity depends on CD99 cell-surface levels (FIGS. 3C-3G). In one example, a cytotoxic effect was not observed in AML cell lines treated with the 10A1 tetravalent antibody, which is likely due to the lower CD99 surface levels in AML cells than T-ALL cells (FIG. 3E). When utilizing the 10A1 ‘constitutive hexamer’ antibody, a cytotoxic effect in both AML and T-ALL cell lines was observed, again with the higher CD99 expression associated with higher antibody mediated cytotoxic activity (FIG. 3G). The strong correlation between cytotoxicity mediated by the 10A1 antibodies and CD99 expression levels indicates that CD99-targeted therapeutics will exhibit minimal toxicity to healthy or low CD99-expressing cells. Furthermore, their efficacy to cells with a lower CD99 level than that of T-ALL cells may be enhanced by changing antibody valency and/or changing the affinity of the Fv unit to CD99, which potentially expand their utility beyond T-ALL.

The preceding Examples reveal that a valency of three is necessary and sufficient for inducing cytotoxicity in T-ALL (FIG. 4B), which indicates a mechanism of action in which the engagement of a minimum of three CD99 molecules induces apoptosis. In contrast, a valency of two is sufficient for achieving strong binding of 10A1 antibodies, as it was observed that the bivalent, trivalent, and tetravalent antibodies have similar apparent K_{D} values to T-ALL cells (FIG. 3B, 4C, 6). This indicates that the higher valency is required for inducing cytotoxicity, but not for engaging the antibodies to the surface of T-ALL cells. Indeed, striking evidence was found that all antigen-binding sites of the tetravalent antibody are engaged with four CD99 molecules when it binds to T-ALL cells (FIG. 3B). Together, these results conclusively establish the basis for the requirements of a valency ≥3 for anti-CD99 antibodies to induce apoptosis by bringing ≥3 CD99 molecules to close proximity on the cell surface.

The requirement of receptor trimerization is reminiscent of the key mechanism that drives signaling of the tumor necrosis factor receptor superfamily (TNFRSF) (Pan et al., “Higher-Order Clustering of the Transmembrane Anchor of DR5 Drives Signaling,” Cell 176:1477-1489 e14 (2019); Zhao et al., “The Diversity and Similarity of Transmembrane Trimerization of TNF Receptors,” Front Cell Dev Biol 8:569684 (2020), each of which is hereby incorporated by reference in its entirety). In a striking parallel to the preceding Examples, a bivalent antibody targeting Fas on a lymphoma cell line does not affect cell viability, whereas crosslinking of the same anti-Fas antibody, which creates a tetravalent molecule, causes cell death (Nakayama et al., “A Humanized Anti-human Fas Antibody, R-125224, Induces Apoptosis in Type I Activated Lymphocytes but Not in Type II Cells,” Int Immunol 18:113-24 (2006), which is hereby incorporated by reference in its entirety). In another example, an anti-OX40 ‘constitutive hexamer’ antibody, but not the bivalent version, exhibited agonistic activity, highlighting the importance of high valency in activating members of the TNFRSF (Yang et al., “Tetravalent Biepitopic Targeting Enables Intrinsic Antibody Agonism of Tumor Necrosis Factor Receptor Superfamily Members,” Mabs 11:996-1011 (2019), which is hereby incorporated by reference in its entirety). Interestingly, the CD99 transmembrane domain contains a sequence similar to the oligomerization motifs of TNFRSF, ΦPxΦ and GxxxG, where Φ is a hydrophobic residue (e.g., Leu, Ile, Val and Phe) and x is any amino acid compatible with partitioning in the membrane (Pan et al., “Higher-Order Clustering of the Transmembrane Anchor of DR5 Drives Signaling,” Cell 176:1477-1489 e14 (2019), which is hereby incorporated by reference in its entirety). Future research will determine whether CD99 also forms a trimer to drive signaling and whether there is a natural ligand that trimerizes CD99.

Antibodies against distinct regions on CD99 have previously been developed and some have been characterized in terms of cytotoxic activity (Pettersen et al., “CD99 Signals Caspase-Independent T Cell Death,” J. Immunol. 166:4931-4942 (2001); Bernard et al., “Apoptosis of Immature Thymocytes Mediated by E2/CD99,” J Immunol 158:2543-50 (1997); Jung et al., “The CD99 Signal Enhances Fas-mediated Apoptosis in the human Leukemic Cell Line, Jurkat,” FEBS Lett 554:478-84 (2003), each of which is hereby incorporated by reference in its entirety). In the preceding Examples, both 10A1 and HO36-1.1 bound residues 63 to 76 and exhibited cytotoxicity (FIG. 2A-B). DN16, mouse IgG1, binds to residues 32 to 39 (Gil et al., “Characterization and Epitope Mapping of Two Monoclonal Antibodies Against Human CD99,” Exp MolMed 34:411-8 (2002), which is hereby incorporated by reference in its entirety), and exhibits cytotoxicity upon crosslinking (Jung et al., “The CD99 Signal Enhances Fas-mediated Apoptosis in the human Leukemic Cell Line, Jurkat,” FEBS Lett 554:478-84 (2003), which is hereby incorporated by reference in its entirety). 0662, a mouse IgG3 that forms non-covalent oligomers (Abdelmoula et al., “Igg3 Is the Major Source of Cryoglobulins in Mice,” J Immunology 143:526-532 (1989); Klaus et al., “CH2 Domain of Mouse IgG3 Governs Antibody Oligomerization, Increases Functional Affinity to Multivalent Antigens and Enhances Hemagglutination,” Front Immunol 9:1096 (2018), each of which is hereby incorporated by reference in its entirety), binds to residues 88 to 97 (Rothermel et al., “High Affinity Cross-reacting mAb Generated by Minimal Mimicry: Implications for the Pathogenesis of Anti-nuclear Autoantibodies and Immunosuppression,” Proc Natl Acad Sci USA 95:3816-20 (1998), which is hereby incorporated by reference in its entirety) and also exhibits cytotoxicity (Bernard et al., “Apoptosis of Immature Thymocytes Mediated by E2/CD99,” J Immunol 158:2543-50 (1997), which is hereby incorporated by reference in its entirety). Taken together, these findings indicate that the 10A1 epitope is not unique in inducing cytotoxicity and that a high valency format of an anti-CD99 antibody is crucial for achieving this effect.

10A1 antibodies with a valency of ≥2 were shown to trigger cell aggregation at concentrations above 200 nM Fv. As discussed above, these antibodies likely ligate a large fraction of CD99 molecules on the cell surface, which suggests a mechanism in which CD99 oligomers promote cell-cell interactions through homotropic interactions across cells. The antibody is unlikely to bridge CD99 molecules on separate cells, as the flow cytometry analysis discussed in the preceding Examples revealed that all the CD99-binding sites on the tetravalent antibody were occupied with the antigen on single cells, leaving no free antigen-binding sites available for bridging cells (FIG. 3B). CD99 was previously shown to be involved in T cell adhesion and rosette formation between T cells and erythrocytes (Bernard et al., “Specifically Triggers Homotypic Aggregation of CD4+CD8+ Thymocytes,” J. Immunol. 154:26-32 (1995); Gelin et al., “The E2 Antigen, a 32 kd Glycoprotein Involved in T-cell Adhesion Processes, Is the MIC2 Gene Product,” EMBO J8:3253-9 (1989), each of which is hereby incorporated by reference in its entirety). It is believed, therefore, that the antibodies described herein may mimic a yet-to-be-identified ligand of CD99 and that this ligand brings together multiple CD99 molecules, inducing cell-cell interaction and ultimately cell apoptosis. A systematic search may discover such a ligand.

Although it is now known from the preceding Examples that a valency of three is sufficient for cytotoxicity (FIG. 4B), a trivalent antibody was not pursued because the production of such an antibody is cumbersome. Instead, the tetravalent antibody was developed, because-unlike the hexamer IgG formats-its valency is independent of antibody concentration, which may contribute to it achieving the highest cytotoxic activity among the formats tested (FIG. 3A, 6). This attribute is beneficial in in vivo applications, as an antibody may be diluted in the blood and in the bone marrow. The 10A1 antibody has a lower IC₅₀ and greater cytotoxic effect on KOPT-K1 than the mouse anti-CD99 IgM, HO36-1.1. Although recent advances have demonstrated the feasibility of manufacturing IgMs for therapeutic uses (Keyt et al., “Structure, Function, and Therapeutic Use of IgM Antibodies,” Antibodies 9(4):53 (2020), which is hereby incorporated by reference in its entirety), it is still difficult to produce IgM antibodies. The tetravalent antibody described herein balances the requirement to achieve high valency and production feasibility. Anti-CD99 antibodies with high valency should contribute to the establishment of targeted therapies for T-ALL and potentially other hematological malignancies.

While Shi et al. (“CAR T Cells Targeting CD99 as an Approach to Eradicate T-Cell Acute Lymphoblastic Leukemia Without Normal Blood Cells Toxicity,” J Hematology & Oncology 14:162 (2021), which is hereby incorporated by reference in its entirety) reported a CAR-T therapy targeting CD99 that showed selective efficacy toward T-ALL cells and in mouse xenograft models, their study and the preceding Examples offer complementary strategies for eradicating T-ALL by targeting CD99. Moreover, the 10A1 antibody and its derivatives may also be used for constructing CAR-T cells and other types of biologics.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. An antibody-based molecule that binds CD99, said antibody-based molecule comprising a heavy chain variable region, wherein said heavy chain variable region comprises:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

2. An antibody-based molecule that binds CD99, said antibody-based molecule comprising one or more amino acid modifications to a heavy chain framework region that enhance stability of the antibody-based molecule that binds CD99, wherein said heavy chain framework region comprises the partial amino acid sequence of:
QVQLQQSGPVLVKPGQTLSLTCAISGDSIS (SEQ ID NO: 54), and
further comprises:
(i) a complementarity-determining region 1 (CDR-H1) comprising an amino acid sequence of any one of SEQ ID NOs: 1-3, or a modified amino acid sequence of any one of SEQ ID NOs: 1-3, said modified sequence having at least 80% sequence identity to any one of SEQ ID NOs: 1-3;
(ii) a complementarity-determining region 2 (CDR-H2) comprising an amino acid sequence of any one of SEQ ID NOs: 4-10, or a modified amino acid sequence of any one of SEQ ID NOs: 4-10, said modified sequences having at least 80% sequence identity to any one of SEQ ID NOs: 4-10; and/or
(iii) a complementarity-determining region 3 (CDR-H3) comprising an amino acid sequence of any one of SEQ ID NOs: 11-14, or a modified amino acid sequence of any one of SEQ ID NO: 11-14, said modified sequence having at least 80% sequence identity to any one of SEQ ID NOs: 11-14.

3. The antibody-based molecule of claim 1 or claim 2, wherein said antibody-based molecule comprises a heavy chain variable region selected from the group consisting of:
(i) a heavy chain variable region comprising the CDR-H1 of SEQ ID NO: 1, the CDR-H2 of SEQ ID NO: 4, and the CDR-H3 of SEQ ID NO: 11;
(ii) a heavy chain variable region comprising the CDR-H1 of SEQ ID NO: 1, the CDR-H2 of SEQ ID NO: 5, and the CDR-H3 of SEQ ID NO: 11;
(iii) a heavy chain variable region comprising the CDR-H1 of SEQ ID NO: 2, the CDR-H2 of SEQ ID NO: 6, and the CDR-H3 of SEQ ID NO: 12; or
(iv) a heavy chain variable region comprising the CDR-H1 of SEQ ID NO: 3, the CDR-H2 of SEQ ID NO: 7, and the CDR-H3 of SEQ ID NO: 13;
(v) a heavy chain variable region comprising the CDR-H1 of SEQ ID NO: 1, the CDR-H2 of SEQ ID NO: 8, and the CDR-H3 of SEQ ID NO: 11;
(vi) a heavy chain variable region comprising the CDR-H1 of SEQ ID NO: 1, the CDR-H2 of SEQ ID NO: 9, and the CDR-H3 of SEQ ID NO: 11;
(vii) a heavy chain variable region comprising the CDR-H1 of SEQ ID NO: 1, the CDR-H2 of SEQ ID NO: 10, and the CDR-H3 of SEQ ID NO: 11;
(viii) a heavy chain variable region comprising the CDR-H1 of SEQ ID NO: 1, the CDR-H2 of SEQ ID NO: 4, and the CDR-H3 of SEQ ID NO: 14; and/or
(ix) a heavy chain variable region comprising the CDR-H1 of SEQ ID NO: 1, the CDR-H2 of SEQ ID NO: 8, and the CDR-H3 of SEQ ID NO: 14.

4. The antibody-based molecule of claim 1 or claim 2, wherein said heavy chain variable region of said antibody-based molecule further comprises human immunoglobulin heavy chain framework regions.

5. The antibody-based molecule of any one of claims 1-4, wherein said molecule comprises:
(i) a heavy chain variable region comprising an amino acid sequence that is at least 80% identical to SEQ ID NO: 28;
(ii) a heavy chain variable region comprising an amino acid sequence that is at least 80% identical to SEQ ID NO: 30;
(iii) a heavy chain variable region comprising an amino acid sequence that is at least 80% identical to SEQ ID NO: 32;
(iv) a heavy chain variable region comprising an amino acid sequence that is at least 80% identical to SEQ ID NO: 34;
(v) a heavy chain variable region comprising an amino acid sequence that is at least 80% identical to SEQ ID NO: 36;
(vi) a heavy chain variable region comprising an amino acid sequence that is at least 80% identical to SEQ ID NO: 37;
(vii) a heavy chain variable region comprising an amino acid sequence that is at least 80% identical to SEQ ID NO: 38;
(viii) a heavy chain variable region comprising an amino acid sequence that is at least 80% identical to SEQ ID NO: 39;
(ix) a heavy chain variable region comprising an amino acid sequence that is at least 80% identical to SEQ ID NO: 40; and/or
(x) a heavy chain variable region comprising an amino acid sequence that is at least 80% identical to SEQ ID NO: 41.

6. The antibody-based molecule of any one of claims 1-5, wherein said antibody-based molecule further comprises a light chain variable region, wherein said light chain variable region comprises:
(i) a complementarity-determining region 1 (CDR-L1) having an amino acid sequence of any one of SEQ ID NOs: 15-18, or a modified amino acid sequence of any one of SEQ ID NO: 15-18, said modified sequence having at least 80% sequence identity to any one of SEQ ID NO: 15-18;
(ii) a complementarity-determining region 2 (CDR-L2) having an amino acid sequence of any one of SEQ ID NOs: 19-22, or a modified amino acid sequence of any one of SEQ ID NO: 19-22, said modified sequence having at least 80% sequence identity to any one of SEQ ID NO: 19-22; and/or
(iii) a complementarity-determining region 3 (CDR-L3) having an amino acid sequence of any one of SEQ ID NOs: 23-27, or a modified amino acid sequence of any one of SEQ ID NO: 23-27, said modified sequence having at least 80% sequence identity to any one of SEQ ID NO: 23-27.

7. The antibody-based molecule of claim 6, wherein said light chain variable region is selected from the group consisting of:
(i) a light chain variable region comprising the CDR-L1 of SEQ ID NO: 15, the CDR-L2 of SEQ ID NO: 19, and the CDR-L3 of SEQ ID NO: 23;
(ii) a light chain variable region comprising the CDR-L1 of SEQ ID NO: 15, the CDR-L2 of SEQ ID NO: 19, and the CDR-L3 of SEQ ID NO: 24;
(iii) a light chain variable region comprising the CDR-L1 of SEQ ID NO: 16, the CDR-L2 of SEQ ID NO: 20, and the CDR-L3 of SEQ ID NO: 25;
(iv) a light chain variable region comprising the CDR-L1 of SEQ ID NO: 17, the CDR-L2 of SEQ ID NO: 21, and the CDR-L3 of SEQ ID NO: 26;
(v) a light chain variable region comprising the CDR-L1 of SEQ ID NO: 18, the CDR-L2 of SEQ ID NO: 19, and the CDR-L3 of SEQ ID NO: 23;
(vi) a light chain variable region comprising the CDR-L1 of SEQ ID NO: 15, the CDR-L2 of SEQ ID NO: 22, and the CDR-L3 of SEQ ID NO: 23; and/or
(vii) a light chain variable region comprising the CDR-L1 of SEQ ID NO: 15, the CDR-L2 of SEQ ID NO: 19, and the CDR-L3 of SEQ ID NO: 27.

8. The antibody-based molecule of claim 7, wherein said light chain variable region of said antibody-based molecule further comprises human immunoglobulin light chain framework regions.

9. The antibody-based molecule of any one of claims 6-8, wherein said molecule comprises:
(i) a light chain variable region comprising an amino acid sequence that is at least 80% identical to SEQ ID NO: 29;
(ii) a light chain variable region comprising an amino acid sequence that is at least 80% identical to SEQ ID NO: 31;
(iii) a light chain variable region comprising an amino acid sequence that is at least 80% identical to SEQ ID NO: 33;
(iv) a light chain variable region comprising an amino acid sequence that is at least 80% identical to SEQ ID NO: 35;
(v) a light chain variable region comprising an amino acid sequence that is at least 80% identical to SEQ ID NO: 42;
(vi) a light chain variable region comprising an amino acid sequence that is at least 80% identical to SEQ ID NO: 43; and/or
(vii) a light chain variable region comprising an amino acid sequence that is at least 80% identical to SEQ ID NO: 44.

10. The antibody-based molecule of claim 9, wherein said antibody-based molecule comprises:
(i) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(ii) a heavy chain variable region comprising the CDR-H1 of SEQ ID NO: 1, the CDR-H2 of SEQ ID NO: 5, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 24;
(iii) a heavy chain variable region comprising the CDR-H1 of SEQ ID NO: 2, the CDR-H2 of SEQ ID NO: 6, and the CDR-H3 of SEQ ID NO: 12, and a light chain variable region comprising the CDR-L1 of SEQ ID NO: 16, the CDR-L2 of SEQ ID NO: 20, and the CDR-L3 of SEQ ID NO: 25;
(iv) a heavy chain variable region comprising the CDR-H1 of SEQ ID NO: 3, the CDR-H2 of SEQ ID NO: 7, and the CDR-H3 of SEQ ID NO: 13, and a light chain variable region comprising the CDR-L1 of SEQ ID NO: 17, the CDR-L2 of SEQ ID NO: 21, and the CDR-L3 of SEQ ID NO: 26;
(v) a heavy chain variable region comprising the CDR-H1 of SEQ ID NO: 1, the CDR-H2 of SEQ ID NO: 82, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(vi) a heavy chain variable region comprising the CDR-H1 of SEQ ID NO: 1, the CDR-H2 of SEQ ID NO: 9, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(vii) a heavy chain variable region comprising the CDR-H1 of SEQ ID NO: 1, the CDR-H2 of SEQ ID NO: 10, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 23;
(viii) a heavy chain variable region comprising the CDR-H1 of SEQ ID NO: 1, the CDR-H2 of SEQ ID NO: 4, and the CDR-H3 of SEQ ID NO: 14, and a light chain variable region comprising the CDR-L1 of SEQ ID NO: 15, the CDR-L2 of SEQ ID NO: 19, and the CDR-L3 of SEQ ID NO: 23;
(ix) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 18, the CDR-L2 of SEQ ID NO: 19, and the CDR-L3 of SEQ ID NO: 23;
(x) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 22, and the CDR-L3 of SEQ ID NO: 23;
(xi) a heavy chain variable region comprising the CDR-H1 of SEQ ID NO: 1, the CDR-H2 of SEQ ID NO: 4, and the CDR-H3 of SEQ ID NO: 11, and a light chain variable region comprising the CDR-L1 of SEQ ID NO: 15, the CDR-L2 of SEQ ID NO: 19, and the CDR-L3 of SEQ ID NO: 27; or
(xii) a heavy chain variable region comprising the CDR-H1 of SEQ ID NO: 1, the CDR-H2 of SEQ ID NO: 8, and the CDR-H3 of SEQ ID NO: 14, and a light chain variable region comprising the CDR-L1 of SEQ ID NO: 15, the CDR-L2 of SEQ ID NO: 19, and the CDR-L3 of SEQ ID NO: 23.

11. The antibody-based molecule of claim 10, wherein said antibody-based molecule further comprises a heavy chain containing a framework region 1 comprising the amino acid sequence of SEQ ID NO: 54.

12. The antibody-based molecule of claim 10 or claim 11, wherein said antibody-based molecule comprises:
(i) a heavy chain variable region comprising an amino acid sequence that is at least 80% identical to SEQ ID NO: 28 and a light chain variable region comprising an amino acid sequence that is at least 80% identical to SEQ ID NO: 29;
(ii) a heavy chain variable region comprising an amino acid sequence that is at least 80% identical to SEQ ID NO: 30 and a light chain variable region comprising an amino acid sequence that is at least 80% identical to SEQ ID NO: 31;
(iii) a heavy chain variable region comprising an amino acid sequence that is at least 80% identical to SEQ ID NO: 32 and a light chain variable region comprising an amino acid sequence that is at least 80% identical to SEQ ID NO: 33;
(iv) a heavy chain variable region comprising an amino acid sequence that is at least 80% identical to SEQ ID NO: 34 and a light chain variable region comprising an amino acid sequence that is at least 80% identical to SEQ ID NO: 35;
(v) a heavy chain variable region comprising an amino acid sequence that is at least 80% identical to SEQ ID NO: 36 and a light chain variable region comprising an amino acid sequence that is at least 80% identical to SEQ ID NO: 29;
(vi) a heavy chain variable region comprising an amino acid sequence that is at least 80% identical to SEQ ID NO: 37 and a light chain variable region comprising an amino acid sequence that is at least 80% identical to SEQ ID NO: 29;
(vii) a heavy chain variable region comprising an amino acid sequence that is at least 80% identical to SEQ ID NO: 38 and a light chain variable region comprising an amino acid sequence that is at least 80% identical to SEQ ID NO: 29;
(viii) a heavy chain variable region comprising an amino acid sequence that is at least 80% identical to SEQ ID NO: 39 and a light chain variable region comprising an amino acid sequence that is at least 80% identical to SEQ ID NO: 29;
(ix) a heavy chain variable region comprising an amino acid sequence that is at least 80% identical to SEQ ID NO: 40 and a light chain variable region comprising an amino acid sequence that is at least 80% identical to SEQ ID NO: 29;
(x) a heavy chain variable region comprising an amino acid sequence that is at least 80% identical to SEQ ID NO: 41 and a light chain variable region comprising an amino acid sequence that is at least 80% identical to SEQ ID NO: 29;
(xi) a heavy chain variable region comprising an amino acid sequence that is at least 80% identical to SEQ ID NO: 28 and a light chain variable region comprising an amino acid sequence that is at least 80% identical to SEQ ID NO: 42;
(xii) a heavy chain variable region comprising an amino acid sequence that is at least 80% identical to SEQ ID NO: 28 and a light chain variable region comprising an amino acid sequence that is at least 80% identical to SEQ ID NO: 43; or
(xiii) a heavy chain variable region comprising an amino acid sequence that is at least 80% identical to SEQ ID NO: 28 and a light chain variable region comprising an amino acid sequence that is at least 80% identical to SEQ ID NO: 44.

13. The antibody-based molecule of any one of claims 1-12, wherein said antibody-based molecule is a monoclonal antibody or binding fragment thereof.

14. The antibody-based molecule of any one of claims 1-12, wherein said antibody-based molecule is a full-length antibody, an epitope-binding fragment of an antibody, or an antibody derivative.

15. The antibody-based molecule of claim 14, wherein said antibody-based molecule is an epitope binding fragment selected from a F(ab) fragment, a F(ab′) fragment, and F(ab′)2 fragment.

16. The antibody-based molecule of claim 14, where said antibody-based molecule is an antibody derivative selected from the group consisting of a scFv, a minibody, a diabody, a triabody, a tribody, and a tetrabody.

17. The antibody-based molecule of claim 1-12, wherein said antibody-based molecule is a bivalent, trivalent, tetravalent, pentavalent, hexavalent, heptavalent, octavalent, nonavalent, decavalent, or dodecavalent antibody-based molecule.

18. The antibody-based molecule of claim 17, wherein said antibody-based molecule is a tetravalent antibody-based molecule.

19. The antibody-based molecule of claim 17, wherein said antibody-based molecule is a dodecavalent antibody-based molecule.

20. The antibody-based molecule of any one of claims 1-12, wherein said antibody-based molecule is an IgG-based molecule.

21. The antibody-based molecule of claim 20, wherein said IgG-based molecule further comprises one or more appended CD99-binding scFv portions.

22. The antibody-based molecule of claim 21, wherein said one or more CD99-binding scFv portions are appended to heavy chains of the IgG-based molecule.

23. The antibody-based molecule of claim 22, wherein the antibody-based molecule is an IgG(H)-scFv or an scFv-(H)IgG.

24. The antibody-based molecule of claim 23, wherein the antibody-based molecule is an IgG(H)-scFv, said IgG(H)-scFv comprising a heavy chain amino acid sequence that is at least 80% identical the amino acid sequence of SEQ ID NO: 46 and a light chain amino acid sequence that is at least 80% identical to the amino acid sequence of SEQ ID NO: 49.

25. The antibody-based molecule of claim 21, wherein said one or more appended CD99-binding scFv portions are appended to the IgG light chain.

26. The antibody-based molecule of claim 20, wherein the antibody-based molecule comprises an Fc domain, said Fc domain comprising one or more amino acid residue substitutions that allow assembly of two or more antibody-based molecules into a molecular complex.

27. The antibody-based molecule of claim 26, wherein the antibody-based molecule comprises an Fc domain comprising amino acid residues 122-451 of SEQ ID NO: 47.

28. The antibody-based molecule of claim 26, wherein the antibody-based molecule comprises an Fc domain comprising amino acid residues 122-451 of SEQ ID NO: 48.

29. An isolated polynucleotide encoding the antibody-based molecule of any one of claims 1-28.

30. A vector comprising the isolated polynucleotide of claim 29.

31. A host cell comprising the vector of claim 30.

32. A pharmaceutical composition comprising:
the antibody-based molecule of any one of claims 1-28, the polynucleotide of claim 29, or the vector of claim 30, and
a pharmaceutically acceptable carrier.

33. A method of inducing apoptosis in a population of CD99-expressing cancer cells, said method comprising:
contacting a population of CD99-expressing cancer cells with a therapeutically effective amount of the antibody-based molecule of any one of claims 1-28, the polynucleotide of claim 29, the vector of claim 30, or the pharmaceutical composition of claim 32 in an amount effective to induce apoptosis in the population of CD99-expressing cancer cells.

34. The method of claim 33, wherein the CD99 expressing cancer cells are from a haematopoietic or lymphoid malignancy.

35. The method of claim 33, wherein the population of CD99-expressing cancer cells is selected from a population of Ewings sarcoma cells, T-cell acute lymphoblastic leukemia (T-ALL) cells, osteosarcoma cells, chondrosarcoma cells, glioma cells, melanoma cells, mesothelioma cells, leukemia cells, prostate cancer cells, liver cancer cells, Hodgkin's lymphoma cells, soft tissue cancer cells, thyroid cancer cells, kidney cancer cells, urinary tract cancer cells, colorectal cancer cells, upper aerodigestive tract cancer cells, B-cell acute lymphoblastic leukemia (B-ALL) cells, non-small cell lung cancer cells, stomach cancer cells, acute myeloid leukemia (AML) cells, endometrial cancer cells, lymphoma cells, breast cancer cells, meningioma cells, chronic myeloid leukemia (CML) cells, ovarian cancer cells, bile duct cancer cells, neuroblastoma cells, pancreatic cancer cells, medulloblastoma cells, esophageal cancer cells, Burkitt's lymphoma cells, small cell lung cancer cells, diffuse large B-cell lymphoma cells, chronic lymphocytic leukemia (CLL) cells, myelodysplastic syndrome (MDS) blast cells, and multiple myeloma cells.

36. The method of any one of claims 33-35, wherein the antibody-based molecule is a bivalent, trivalent, tetravalent, or dodecavalent antibody-based molecule.

37. A method of treating a CD99-expressing cancer in a subject, said method comprising:
administering, to a subject having a CD99-expressing cancer, an antibody-based molecule of any one of claims 1-28, the polynucleotide of claim 29, the vector of claim 30, or the pharmaceutical composition of claim 32 in an amount effective to treat the cancer in the subject.

38. The method of claim 37, wherein the CD99-expressing cancer is a haematopoietic or lymphoid malignancy.

39. The method of claim 37, wherein the CD99-expressing cancer is selected from Ewings sarcoma, T-cell acute lymphoblastic leukemia (T-ALL), osteosarcoma, chondrosarcoma, glioma, melanoma, mesothelioma, leukemia, prostate cancer, liver cancer, Hodgkin's lymphoma, soft tissue cancer, thyroid cancer, kidney cancer, urinary tract cancer, colorectal cancer, upper aerodigestive tract cancer, B-cell acute lymphoblastic leukemia (B-ALL), non-small cell lung cancer, stomach cancer, acute myeloid leukemia (AML), endometrial cancer, lymphoma, breast cancer, meningioma, chronic myeloid leukemia (CML), ovarian cancer, bile duct cancer, neuroblastoma, pancreatic cancer, medulloblastoma, esophageal cancer, Burkitt's lymphoma, small cell lung cancer, diffuse large B-cell lymphoma, chronic lymphocytic leukemia (CLL), myelodysplastic syndrome (MDS), or multiple myeloma.

40. The method of any one of claims 37-39, wherein the antibody-based molecule is a bivalent, trivalent, tetravalent, or dodecavalent antibody-based molecule.
